(19) United States
(12) Patent Application Publication
(10) Pub. No.: US 2025/0082589 A1
(43) Pub. Date: Mar. 13, 2025

(54) PHENYLETHYLIDENEHYDRAZINE DIMERS AND METHODS OF USING SAME

(71) Applicant: NEURAWELL THERAPEUTICS, Boca Raton, FL (US)

(72) Inventors: Magid Abraham, Boca Raton, FL (US); Mark A. Ashwell, Carlisle, MA (US); Steve Stahl, Henderson, NV (US)

(21) Appl. No.: 18/720,402

(22) PCT Filed: Dec. 16, 2022
(86) PCT No.: PCT/US2022/053108

§ 371 Date: Jun. 14, 2024

Related U.S. Application Data
(60) Provisional application No. 63/290,634, filed on Dec. 16, 2021.

(51) Int. Cl. A61K 31/15; A61K 31/506

(52) U.S. Cl. CPC A61K 31/15; A61K 31/506

(57) ABSTRACT

The invention provides compositions and methods for the treatment of treatment-resistant depression and other psychological disorders. In particular, the invention provides phenylethylidenehydrazine dimers, and methods, devices, and kits that incorporate the dimers. This abstract is intended as a scanning tool for purposes of searching in the particular art and is not intended to be limiting of the present invention.

### CROSS-REFERENCE TO RELATED APPLICATIONS

This application claims the benefit of U.S. Application No. 63/290,634, filed on Dec. 16, 2021, the contents of which are incorporated herein by reference in their entirety.

### BACKGROUND

Depression is one of the most common mental disorders in the U.S. Depression rates are on the rise, and over 300 million people are affected worldwide. However, current treatments of depression suffer from unwanted side effects, and in some cases, they are not effective for treating all cases. It is estimated that antidepressant medication does not fully work for ˜30% to 45% of people. Such patients are classified as having a Treatment Resistant Depression or TRD. It is estimated that 70% of the suicides are among this population. In addition, they tend to have more health issues, to the point that their life expectancy is reduced by 10 years. The health care cost per TRD patient is about 10 times the cost of a normal person, and the aggregate cost to the US economy alone is estimated to be at least $30 Billion.

Current treatment options for depression include monotherapy or combination therapy with various classes of drugs such as, for example, serotonin specific reuptake inhibitors (SSRIs), serotonin noradrenergic reuptake inhibitors (SNRIs), norepinephrine-dopamine reuptake inhibitors (NDRIs), mono-amine oxidase inhibitors (MAOIs), tricyclic antidepressants (TCAs), anti-psychotics, “natural products” (e.g., Kava-Kava, St. John's Wort), dietary supplement (e.g., s-adenosylmethionine), and others. Unfortunately, in the clinic, 40-50% of depressed patients who are initially prescribed antidepressant therapy do not experience a timely remission of depression symptoms. This group typifies level 1 treatment-resistant depression, which is characterized by a failure to demonstrate an “adequate” response to an “adequate” treatment trial (that is, sufficient intensity of treatment for sufficient duration). Moreover, about approximately 30% of depressed patients remain partially or totally treatment-resistant to at least two antidepressant treatments including combination treatments.

Phenelzine (PLZ) is a non-selective MAOI commonly used to treat depression and panic disorder. As expected, PLZ increases brain levels of dopamine, norepinephrine, and serotonin. Interestingly, PLZ also elevates brain levels of γ-aminobutyric acid (GABA), an inhibitory neurotransmitter that provide the basis for such anxiety drugs as benzodiazepines. Previous studies have suggested that these increases may contribute to the anxiolytic effects of PLZ. Despite the therapeutic relevance of MAOI's such as PLZ, their use in the clinic has remained limited due to the strict dietary restrictions that patients must follow to avoid potentially dangerous hypertensive crises. Specifically, patients must avoid food and beverages such as cheese, wine, and beer, which are known to contain an excess of tyramine, a naturally occurring substance that shows increased levels in food with fermentation and aging (Thase, et al., 1995). When excessive amounts of tyramine are ingested due to failure to follow dietary restrictions, dangerous or life-threatening blood pressure increases may result (GlaxoSmithKline, 2008; Gillman, 2017).

More recently, it was discovered that phenylethylidenehydrazine (PEH), a metabolite of PEH, inhibits the GABA catabolic enzyme GABA-transaminase and increases brain levels of GABA but offers only weak and transient inhibition of monoamine oxidase (resulting in negligible increase of serotonin, norepinephrine, and dopamine). See, e.g., Matveychuk et al. (2013) J Neural. Transm. 120: 987-996. Thus, it was theorized that antidepressant therapies using PEH could beneficially avoid the negative side effects of MAOIs. Unfortunately, PEH suffers from drawbacks of its own, being extremely unstable and difficult to purify. Accordingly, there remains a need for compositions and methods for treating psychological disorders such as depression that avoid the limitations of MAOIs, while also being amenable to use in a clinical setting (e.g., stable, easily purified). These needs and others are met by the present invention.

### SUMMARY

In accordance with the purpose(s) of the invention, as embodied and broadly described herein, the invention, in one aspect, relates to methods for treating psychological disorders such as, for example, severe anxiety disorders (e.g., generalized anxiety disorder (GAD), panic disorder, depression, treatment-resistant depression, depression with severe anxiety, and bipolar disorder with severe anxiety) using phenylethylidenehydrazine (PEH) dimers, alone or in combination with an antidepressant, and methods of making and using same. Without wishing to be bound by theory, the disclosed compositions and methods introduce a treatment that potentiates GABA levels in the brain, without the risk of serious drug and dietary interactions associated with the use of phenelzine. Further, the disclosed compounds offer improved stability compared to alternative formulations, and can be readily purified, which adds to their therapeutic utility.

Thus, disclosed are pharmaceutical compositions comprising a therapeutically effective amount of a compound having a structure represented by a formula:

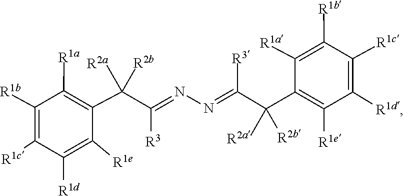

wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C₁-C₄ alkyl, C₂-C₄ alkenyl, C₁-C₄ haloalkyl, C₁-C₄ cyanoalkyl, C₁-C₄ hydroxyalkyl, C₁-C₄ haloalkoxy, C₁-C₄ alkoxy, C₁-C₄ alkylamino, (C₁-C₄)(C₁-C₄) dialkylamino, and C₁-C₄ aminoalkyl; wherein each of R^{2a}, R^{2a′}, R^{2b}, and R^{2b′} is independently selected from hydrogen, halogen, —OH, —NH₂, C₁-C₄ alkyl, and C₂-C₄ alkenyl; and wherein each of R³ and R^{3′} is independently selected from hydrogen and C₁-C₄ alkyl, or a pharmaceutically acceptable salt thereof, and a pharmaceutically acceptable carrier. In a further aspect, the compound is laterally symmetric.

Also disclosed are pharmaceutical compositions comprising a therapeutically effective amount of a compound having a structure:

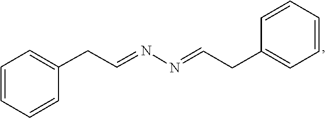 [SMILES: C(/Cc1ccccc1)=N\N=C\Cc1ccccc1]

or a pharmaceutically acceptable salt thereof, and a pharmaceutically acceptable carrier.

Also disclosed are pharmaceutical compositions comprising a therapeutically effective amount of a compound having a structure represented by a formula:

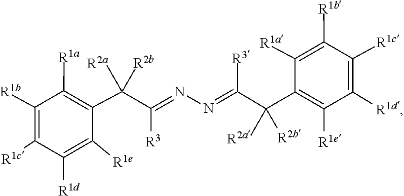

wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C₁-C₄ alkyl, C₂-C₄ alkenyl, C₁-C₄ haloalkyl, C₁-C₄ cyanoalkyl, C₁-C₄ hydroxyalkyl, C₁-C₄ haloalkoxy, C₁-C₄ alkoxy, C₁-C₄ alkylamino, (C₁-C₄)(C₁-C₄) dialkylamino, and C₁-C₄ aminoalkyl; wherein each of R^{2a}, R^{2a′}, R^{2b}, and R^{2b′} is independently selected from hydrogen, halogen, —OH, —NH₂, C₁-C₄ alkyl, and C₂-C₄ alkenyl; and wherein each of R³ and R^{3′} is independently selected from hydrogen and C₁-C₄ alkyl, or a pharmaceutically acceptable salt thereof, and an antidepressant, a biocompatible polymer, and a pharmaceutically acceptable carrier.

Also disclosed are pharmaceutical compositions comprising a therapeutically effective amount of a compound having a structure:

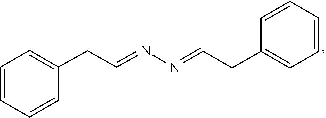 [SMILES: C(/Cc1ccccc1)=N\N=C\Cc1ccccc1]

or a pharmaceutically acceptable salt thereof and an antidepressant, a biocompatible polymer, and a pharmaceutically acceptable carrier.

Also disclosed are methods for treating a psychological disorder in a subject in need thereof, the method comprising administering to the subject an effective amount of a compound having a structure represented by a formula:

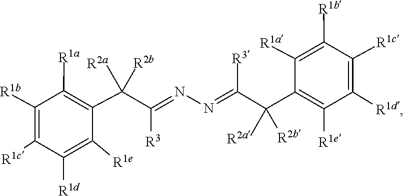

wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C₁-C₄ alkyl, C₂-C₄ alkenyl, C₁-C₄ haloalkyl, C₁-C₄ cyanoalkyl, C₁-C₄ hydroxyalkyl, C₁-C₄ haloalkoxy, C₁-C₄ alkoxy, C₁-C₄ alkylamino, (C₁-C₄)(C₁-C₄) dialkylamino, and C₁-C₄ aminoalkyl; wherein each of R^{2a}, R^{2a′}, R^{2b}, and R^{2b′} is independently selected from hydrogen, halogen, —OH, —NH₂, C₁-C₄ alkyl, and C₂-C₄ alkenyl; and wherein each of R³ and R^{3′} is independently selected from hydrogen and C₁-C₄ alkyl, or a pharmaceutically acceptable salt thereof.

Also disclosed are methods for treating a psychological disorder in a subject in need thereof, the method comprising administering to the subject an effective amount of a compound having a structure:

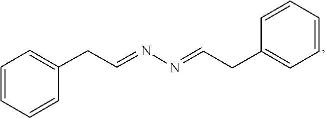 [SMILES: C(/Cc1ccccc1)=N\N=C\Cc1ccccc1]

or a pharmaceutically acceptable salt thereof.

Also disclosed are methods for treating a psychological disorder in a subject in need thereof, the method comprising administering to the subject an effective amount of a compound having a structure represented by a formula:

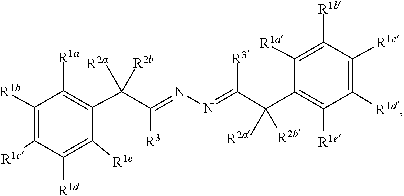

wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C₁-C₄ alkyl, C₂-C₄ alkenyl, C₁-C₄ haloalkyl, C₁-C₄ cyanoalkyl, C₁-C₄ hydroxyalkyl, C₁-C₄ haloalkoxy, C₁-C₄ alkoxy, C₁-C₄ alkylamino, (C₁-C₄)(C₁-C₄) dialkylamino, and C₁-C₄ aminoalkyl; wherein each of R^{2a}, R^{2a′}, R^{2b}, and R^{2b′} is independently selected from hydrogen, halogen, —OH, —NH₂, C₁-C₄ alkyl, and C₂-C₄ alkenyl; and wherein each of R³ and R^{3′} is independently selected from hydrogen and C₁-C₄ alkyl, or a pharmaceutically acceptable salt thereof, and an antidepressant, wherein the subject has not responded to at least one adequate antidepressant treatment prior to the administering step.

Also disclosed are methods for treating a psychological disorder in a subject in need thereof, the method comprising administering to the subject an effective amount of a compound having a structure:

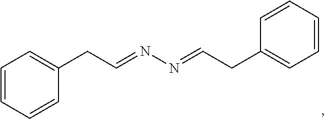 [SMILES: C(/Cc1ccccc1)=N\N=C\Cc1ccccc1]

or a pharmaceutically acceptable salt thereof, and an antidepressant, wherein the subject has not responded to at least one adequate antidepressant treatment prior to the administering step.

Also disclosed are kits comprising a compound having a structure represented by a formula:

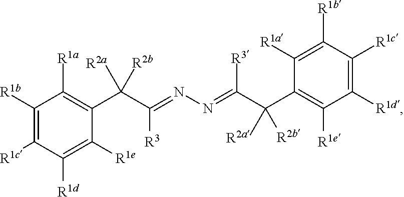

wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C₁-C₄ alkyl, C₂-C₄ alkenyl, C₁-C₄ haloalkyl, C₁-C₄ cyanoalkyl, C₁-C₄ hydroxyalkyl, C₁-C₄ haloalkoxy, C₁-C₄ alkoxy, C₁-C₄ alkylamino, (C₁-C₄)(C₁-C₄) dialkylamino, and C₁-C₄ aminoalkyl; wherein each of R^{2a}, R^{2a′}, R^{2b}, and R^{2b′} is independently selected from hydrogen, halogen, —OH, —NH₂, C₁-C₄ alkyl, and C₂-C₄ alkenyl; and wherein each of R³ and R^{3′} is independently selected from hydrogen and C₁-C₄ alkyl, or a pharmaceutically acceptable salt thereof, and one or more selected from: (a) an antidepressant; (b) a device for delivering a medicament orally or intranasally; (c) a microneedle array or a transdermal patch; and (d) instructions for treating a psychological disorder.

Also disclosed are kits comprising a compound having a structure:

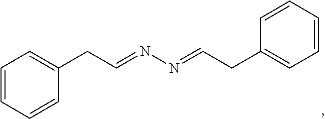 [SMILES: C(/Cc1ccccc1)=N\N=C\Cc1ccccc1]

or a pharmaceutically acceptable salt thereof, and one or more selected from: (a) an antidepressant; (b) a device for delivering a medicament orally or intranasally; (c) a microneedle array or a transdermal patch; and (d) instructions for treating a psychological disorder.

Also disclosed are devices comprising: (a) a compound having a structure represented by a formula:

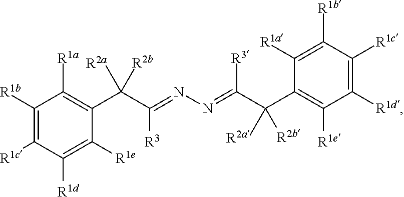

wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C₁-C₄ alkyl, C₂-C₄ alkenyl, C₁-C₄ haloalkyl, C₁-C₄ cyanoalkyl, C₁-C₄ hydroxyalkyl, C₁-C₄ haloalkoxy, C₁-C₄ alkoxy, C₁-C₄ alkylamino, (C₁-C₄)(C₁-C₄) dialkylamino, and C₁-C₄ aminoalkyl; wherein each of R^{2a}, R^{2a′}, R^{2b}, and R^{2b′} is independently selected from hydrogen, halogen, —OH, —NH₂, C₁-C₄ alkyl, and C₂-C₄ alkenyl; and wherein each of R³ and R^{3′} is independently selected from hydrogen and C₁-C₄ alkyl, or a pharmaceutically acceptable salt thereof; (b) a microneedle array or a transdermal patch; and (c) optionally, a transdermal agent.

Also disclosed are devices comprising: (a) a compound having a structure:

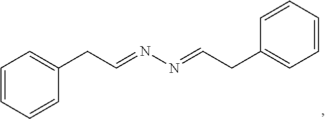 [SMILES: C(/Cc1ccccc1)=N\N=C\Cc1ccccc1]

or a pharmaceutically acceptable salt thereof; (b) a microneedle array or a transdermal patch; and (c) optionally, a transdermal agent.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE DRAWINGS

The accompanying figures, which are incorporated in and constitute a part of this specification, illustrate several aspects and together with the description serve to explain the principles of the invention.

FIG. 1A-C show representative UPLC chromatograms obtained for reaction 1830-012, azine synthesis at different time points.

FIG. 2 shows a representative plot disclosing % azine as determined by ¹H NMR in ACN-d₃, CDCl₃, MeOD, C₆D₆, THF-d₈, CDCl₃ (1 mg), and Py-d₅. Data was recorded directly after preparation (1), 4 hours after preparation (2), and 3 days after preparation (3).

FIG. 3A and FIG. 3B show representative ¹H NMR spectra obtained in CDCl₃ for the azine product of 1830-012 after storage at room temperature for 3 days. Spectra are shown for the reaction 3 days after sample preparation (FIG. 3A) and a newly prepared sample after 3 days (FIG. 3B). The numbered protons on the reference compounds are matched to the corresponding NMR signals.

FIG. 4A and FIG. 4B show representative data illustrating 2D Thin Layer Chromatography (TLC Silica gel 60 NH₂F₂₅₄s) analysis of the PEH product of 1830-011 (FIG. 4A) and the azine product of 1830-012 (FIG. 4B). The solvent used was Hexane: Ethyl acetate at a ratio of 6:1.

FIG. 5 shows representative UPLC chromatograms for the azine product formed in 1830-0012 and subjected to crystallization. The top panel shows the chromatogram for the precipitate in ACN. The bottom panel shows the chromatogram for the filtrate in ACN. Both PEH and azine were detected in both runs.

FIG. 6A-C show a representative chromatogram (FIG. 6A) and a representative ¹H NMR spectra in CDCl₃ (FIG. 6B) and DMSO-d₆ (FIG. 6C) for the product formed in reaction 1830-013. The numbered protons on the reference compounds are matched to the corresponding NMR signals. PEH and Azine were detected in the UPLC, but were not detected in the CDCl₃ NMR.

FIG. 7A and FIG. 7B shows representative data illustrating the results of pH stability studies for the azine product formed in 1830-013 at pH 9.2 (FIG. 7A) and pH 7.4 (FIG. 7B). The amount of the azine (FIG. 7A) and the amount of the azine, the aldehyde, and PEH (FIG. 7B) expressed as percent was plotted over time. No signals of aldehyde and PEH were observed at pH 9.2.

FIG. 8A-D show a representative chromatogram (FIG. 8A) and a representative ¹H NMR spectra in CDCl₃ (FIG. 38B), DMSO-d₆ (FIG. 38C), and MeOD (FIG. 38D) for the product formed in reaction 1830-014. The numbered protons on the reference compounds are matched to the corresponding NMR signals. PEH and Azine were detected in the UPLC, but were not detected in the CDCl₃ NMR.

FIG. 9 shows representative ¹H NMR spectra in CDCl₃ for the azine products formed in 1830-014 (top panel and middle panel, white precipitate) and 1803-015 (bottom panel). The numbered protons on the reference compounds are matched to the corresponding NMR signals.

FIG. 10A and FIG. 10B show representative data illustrating the LCMS results for the yellow precipitate azine product formed in 1830-014 directly after dissolution (FIG. 10A) and 27 hours after dissolution (FIG. 10B). The top panels are the chromatograms. The middle panels are mass spectra that show the azine was detected directly after dissolution. The bottom panel are mass spectra that show the azine was also detected 27 hours after dissolution.

FIG. 11A-C show representative UPLC (FIG. 11A), ¹H NMR (FIG. 11B), and preparative HPLC (FIG. 11C) spectra obtained for reaction 1830-015, azine synthesis.

FIG. 12A-D show representative data pertaining to the characterization and purity of the product formed in 1830-015. Specifically, FIG. 12A shows representative chromatograms of the azine product after preparative HPLC. FIG. 12B shows a representative ¹H NMR spectrum in DMSO-d₆ for the first fraction of the azine product formed after lyophilization. FIG. 12C and FIG. 12D show representative data illustrating the LCMS results for the first fraction of the azine product formed in 1830-015 in solution (FIG. 12C) and after lyophilization (FIG. 12D). The top panels of FIG. 12C and FIG. 12D are the chromatograms. The middle panels are mass spectra that show the azine was detected in the first fraction in solution. The bottom panels are mass spectra that show the azine was also detected in the first fraction after lyophilization.

FIG. 13A-E show representative UPLC chromatograms obtained for reactions 1830-001 (acidic and basic conditions), 1830-002, and 1830-003 (acidic and basic conditions), azine syntheses.

FIG. 14A-C show representative ¹H NMR spectra obtained for reaction 1830-003A and 1830-003B.

FIG. 15A and FIG. 15B show representative UPLC chromatograms obtained for samples 1830-004 and 1830-006, respectively.

FIG. 16A and FIG. 16B show representative ¹H NMR spectra obtained for samples 1830-004 and 1830-006, respectively.

FIG. 17A-F show representative spectral data for sample 1830-007. Specifically, FIG. 17A and FIG. 17B show representative spectra illustrating mass spectrometry (top panels) and chromatography (bottom panels) analysis of the product of the reaction of 1830-007 after extraction and evaporation (FIG. 17A) and after storage at −20° C. under Ar for 3 days. The mass spectrometry confirmed the presence of PEH and the azine at [M+H]⁺=135.6 and 237.8, respectively. FIG. 17C-F show representative data obtained for Fraction 1 (FIG. 17C and FIG. 17D) and Fraction 2 (FIG. 17E and FIG. 17F) of the distillation of reaction product 1830-007. The numbered protons on the reference compounds are matched to the corresponding NMR signals. PEH and the azine were both observed in Fraction 1; however, the azine ([M+H]⁺=237.8) was only detected via mass spectrometry in Fraction 2. No signals due to the azine or PEH were detected in the NMR spectrum.

FIG. 18A-C show representative UPLC chromatograms obtained for reaction 1830-011, PEH synthesis.

FIG. 19 shows a representative plot disclosing the % PEH in the product of 1830-011 as determined by ¹H NMR in ACN-d₃, CDCl₃, MeOD, C₆D₆, THF-d₈, CDCl₃ (1 mg), and Py-d₅. Data was recorded directly after preparation (1), 4 hours after preparation (2), and 3 days after preparation (3).

FIG. 20A and FIG. 20B show representative ¹H NMR spectra obtained in CDCl₃ for the PEH product of 1830-011 after storage at room temperature for 3 days. Spectra are shown for the reaction 3 days after sample preparation (FIG. 20A) and a newly prepared sample after 3 days (FIG. 20B). The numbered protons on the reference compounds are matched to the corresponding NMR signals.

FIG. 21 shows a representative preparative HPLC chromatogram obtained from the PEH product formed in 1830-011. Two major peaks were observed at R.T. 7.6 min and 8.3 min.

FIG. 22A-D show representative data pertaining to batch 1830-011. Specifically, FIG. 22A shows representative chromatograms of the PEH product formed in 1830-011 after preparative HPLC, where the top panel shows the first fraction in solution, the middle panel shows the first fraction after lyophilization, and the bottom panel shows the second fraction in solution. FIG. 22B shows a representative ¹H NMR spectrum in CDCl₃ for the first fraction of the PEH product formed in 1830-011 after lyophilization. The numbered protons on the reference compounds are matched to the corresponding NMR signals. FIG. 22C and FIG. 22D show representative data illustrating the LCMS results for the first fraction of the PEH product formed in 1830-011 in solution (FIG. 22C) and after lyophilization (FIG. 22D).

FIG. 23A and FIG. 23B show representative spectral data for the PEH product formed in 1830-016.

FIG. 24 shows representative data for the PEH product formed in 1830-018.

FIG. 25A and FIG. 25C show representative data illustrating the results of pH stability studies for the azine product formed in 1830-018 at pH 9.2 (FIG. 25A) and pH 7.4 (FIG. 25B).

FIG. 26A-C show representative LCMS (FIG. 26A and FIG. 26B) and ¹H NMR (FIG. 26C) spectral data for the azine product of batches 1830-021 and 1830-022.

FIG. 27 shows representative data illustrating the stability of PEH over time.

FIG. 28 shows a representative ¹H NMR spectra of PEH after concentration, with two isomers visible.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

As used in the specification and the appended claims, the singular forms “a,” “an” and “the” include plural referents unless the context clearly dictates otherwise. Thus, for example, reference to “an antidepressant,” “a psychological disorder,” or “a subject” includes mixtures of two or more such antidepressants, psychological disorders, or subjects, and the like.

As used in the specification and in the claims, the term “comprising” can include the aspects “consisting of” and “consisting essentially of.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig, or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage form can comprise a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions, which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “azine” refers to a dimer of phenylethylidenehydrazine, or a derivative thereof, having a core structure:

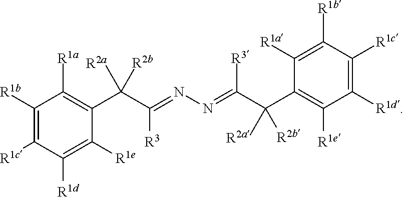

Exemplary azines include, but are not limited to, (1E,2E)-1,2-bis(2-phenylethylidene)hydrazine, having a structure:

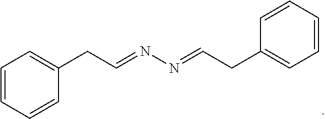 [SMILES: C(/Cc1ccccc1)=N\N=C\Cc1ccccc1]

Additional examples of azines are disclosed elsewhereherein.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, and amides, salts of esters or amides, and N-oxides of a parent compound.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radioactively labeled forms, isomers, solvates and combinations thereof. The “combinations” mentioned in this context are refer to derivatives falling within at least two of the groups: pharmaceutically acceptable salts, prodrugs, deuterated forms, radioactively labeled forms, isomers, and solvates. Examples of radioactively labeled forms include compounds labeled with tritium, phosphorous-32, iodine-129, carbon-11, fluorine-18, and the like.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbomyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or -OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aromatic group” as used herein refers to a ring structure having cyclic clouds of delocalized π electrons above and below the plane of the molecule, where the π clouds contain (4n+2) π electrons. A further discussion of aromaticity is found in Morrison and Boyd, Organic Chemistry, (5th Ed., 1987), Chapter 13, entitled “Aromaticity,” pages 477-497, incorporated herein by reference. The term “aromatic group” is inclusive of both aryl and heteroaryl groups.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The terms “pseudohalide,” “pseudohalogen,” or “pseudohalo,” as used herein can be used interchangeably and refer to functional groups that behave substantially similar to halides. Such functional groups include, by way of example, cyano, thiocyanato, azido, trifluoromethyl, trifluoromethoxy, perfluoroalkyl, and perfluoroalkoxy groups.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl”, “heteroaryl”, “bicyclic heterocycle” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula—SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA′, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A'S(O)A², where A¹ and A² can be, independently, an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “R,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the invention may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{•} together with their intervening atoms), are independently halogen, —(CH₂)O₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiRR^{•}₃, —OSiRR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of RT are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Protective Groups in Organic Synthesis,” T. W. Greene, P. G. M. Wuts, Wiley-Interscience, 1999).

The term “organic residue” defines a carbon containing residue, i.e., a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited alkyl or substituted alkyls, alkoxy or substituted alkoxy, mono or di-substituted amino, amide groups, etc. Organic residues can preferably comprise 1 to 18 carbon atoms, 1 to 15, carbon atoms, 1 to 12 carbon atoms, 1 to 8 carbon atoms, 1 to 6 carbon atoms, or 1 to 4 carbon atoms. In a further aspect, an organic residue can comprise 2 to 18 carbon atoms, 2 to 15, carbon atoms, 2 to 12 carbon atoms, 2 to 8 carbon atoms, 2 to 4 carbon atoms, or 2 to 4 carbon atoms.

A very close synonym of the term “residue” is the term “radical,” which as used in the specification and concluding claims, refers to a fragment, group, or substructure of a molecule described herein, regardless of how the molecule is prepared. For example, a 2,4-thiazolidinedione radical in a particular compound has the structure:

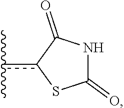

regardless of whether thiazolidinedione is used to prepare the compound. In some embodiments the radical (for example an alkyl) can be further modified (i.e., substituted alkyl) by having bonded thereto one or more “substituent radicals.” The number of atoms in a given radical is not critical to the present invention unless it is indicated to the contrary elsewhere herein.

“Organic radicals,” as the term is defined and used herein, contain one or more carbon atoms. An organic radical can have, for example, 1-26 carbon atoms, 1-18 carbon atoms, 1-12 carbon atoms, 1-8 carbon atoms, 1-6 carbon atoms, or 1-4 carbon atoms. In a further aspect, an organic radical can have 2-26 carbon atoms, 2-18 carbon atoms, 2-12 carbon atoms, 2-8 carbon atoms, 2-6 carbon atoms, or 2-4 carbon atoms. Organic radicals often have hydrogen bound to at least some of the carbon atoms of the organic radical. One example, of an organic radical that comprises no inorganic atoms is a 5, 6, 7, 8-tetrahydro-2-naphthyl radical. In some embodiments, an organic radical can contain 1-10 inorganic heteroatoms bound thereto or therein, including halogens, oxygen, sulfur, nitrogen, phosphorus, and the like. Examples of organic radicals include but are not limited to an alkyl, substituted alkyl, cycloalkyl, substituted cycloalkyl, mono-substituted amino, di-substituted amino, acyloxy, cyano, carboxy, carboalkoxy, alkylcarboxamide, substituted alkylcarboxamide, dialkylcarboxamide, substituted dialkylcarboxamide, alkylsulfonyl, alkylsulfinyl, thioalkyl, thiohaloalkyl, alkoxy, substituted alkoxy, haloalkyl, haloalkoxy, aryl, substituted aryl, heteroaryl, heterocyclic, or substituted heterocyclic radicals, wherein the terms are defined elsewhere herein. A few non-limiting examples of organic radicals that include heteroatoms include alkoxy radicals, trifluoromethoxy radicals, acetoxy radicals, dimethylamino radicals and the like.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷, ³⁵S, ¹⁸F, and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labeled compounds of the present invention, for example those into which radioactive isotopes such as ³H and ¹⁴C are incorporated, are useful in drug and/or substrate tissue distribution assays. Tritiated, i.e., 3H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present invention and prodrugs thereof can generally be prepared by carrying out the procedures below, by substituting a readily available isotopically labeled reagent for a non-isotopically labeled reagent.

The compounds described in the invention can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the invention to form solvates and hydrates. Unless stated to the contrary, the invention includes all such possible solvates.

The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Examples of co-crystals include p-toluenesulfonic acid and benzenesulfonic acid.

It is also appreciated that certain compounds described herein can be present as an equilibrium of tautomers. For example, ketones with an α-hydrogen can exist in an equilibrium of the keto form and the enol form.

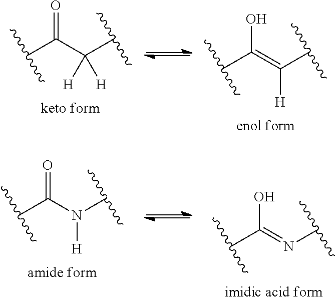 [SMILES: *CC(*)=O ; */C=C(/*)O ; *NC(*)=O ; */N=C(/*)O]

Likewise, amides with an N-hydrogen can exist in an equilibrium of the amide form and the imidic acid form. As another example, pyrazoles can exist in two tautomeric forms, N¹-unsubstituted, 3-A³ and N¹-unsubstituted, 5-A³ as shown below.

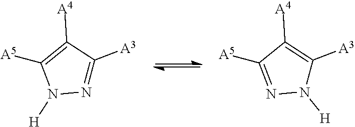

Unless stated to the contrary, the invention includes all such possible tautomers.

It is known that chemical substances form solids which are present in different states of order which are termed polymorphic forms or modifications. The different modifications of a polymorphic substance can differ greatly in their physical properties. The compounds according to the invention can be present in different polymorphic forms, with it being possible for particular modifications to be metastable. Unless stated to the contrary, the invention includes all such possible polymorphic forms.

In some aspects, a structure of a compound can be represented by a formula:

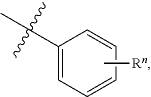

which is understood to be equivalent to a formula:

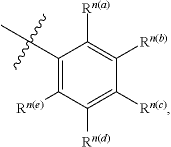

wherein n is typically an integer. That is, Rⁿ is understood to represent five independent substituents, R^{n(a)}, R^{n(b)}, R^{n(c)}, R^{n(d)}, R^{n(e)}. By “independent substituents,” it is meant that each R substituent can be independently defined. For example, if in one instance R^{n(a)} is halogen, then R^{n(b)} is not necessarily halogen in that instance.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B—F, C-D, C-E, and C—F are considered disclosed. Likewise, any subset or combination of these is also disclosed. Thus, for example, the sub-group of A-E, B—F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Compounds

In one aspect, the invention relates to compounds useful in treating psychological disorders such as, for example, severe anxiety disorders (e.g., generalized anxiety disorder (GAD), panic disorder, depression, treatment-resistant depression, depression with severe anxiety, and bipolar disorder with severe anxiety).

In one aspect, the compounds of the invention are useful in treating a psychological disorder in a mammal. In a further aspect, the compounds of the invention are useful in treating a psychological disorder in a human.

In one aspect, the compounds of the invention are useful in the treatment of psychological disorders, as further described herein.

It is contemplated that each disclosed derivative can be optionally further substituted. It is also contemplated that any one or more derivative can be optionally omitted from the invention. It is understood that a disclosed compound can be provided by the disclosed methods. It is also understood that the disclosed compounds can be employed in the disclosed methods of using.

### 1. Structure

In one aspect, disclosed are compounds having a structure represented by a formula:

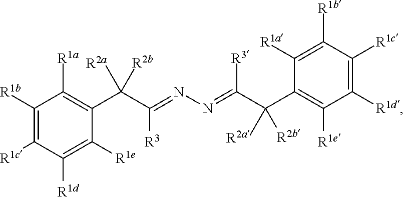

wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a}, R^{2a′}, R^{2b}, and R^{2b′} is independently selected from hydrogen, halogen, —OH, —NH₂, C1-C4 alkyl, and C2-C4 alkenyl; and wherein each of R³ and R^{3′} is independently selected from hydrogen and C1-C4 alkyl, or a pharmaceutically acceptable salt thereof, and a pharmaceutically acceptable carrier. In a further aspect, the compound is laterally symmetric.

In various aspects, the compound has a structure represented by a formula;

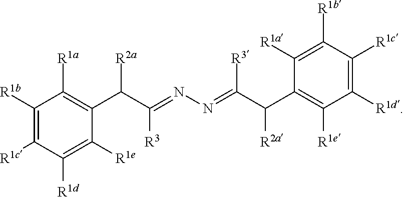

In various aspects, the compound has a structure represented by a formula;

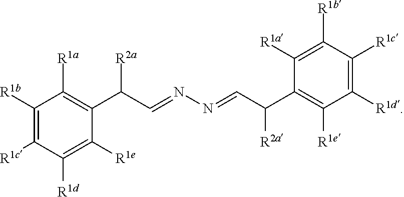

In various aspects, the compound has a structure represented by a formula;

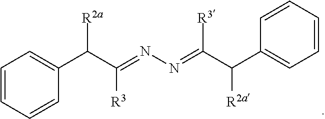

In various aspects, the compound has a structure represented by a formula;

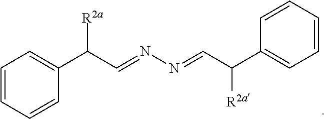

In various aspects, the compound has a structure represented by a formula;

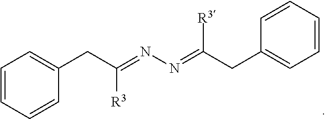

In various aspects, the compound has a structure represented by a formula;

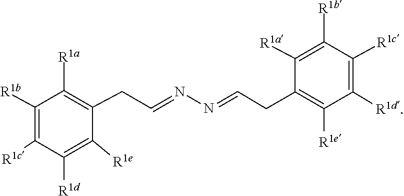

In various aspects, the compound is:

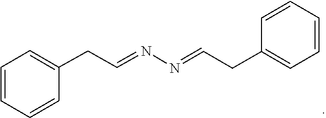 [SMILES: C(/Cc1ccccc1)=N\N=C\Cc1ccccc1]

a. R^{1A}, R^{1B}, R^{1C}, R^{1D}, R^{1E}, R^{1A′}, R^{1B′}, R^{1C′}, R^{1D′}, and R^{1E′} Groups

In one aspect, each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In various aspects, each of R^{1a}, R^{1b}, R^{1c}, R^{1d}, R^{1e}, R^{1a′}, R^{1b′}, R^{1c′}, R^{1d′}, and R^{1e′} is hydrogen.

In various aspects, R^{1a} is the same as R^{1a′}. In a further aspect, R^{1b} is the same as R^{1b′}. In a still further aspect, R^{1c} is the same as R^{1c′}. In yet a further aspect, R^{1d} is the same as R^{1d′}. In an even further aspect, R^{1e} is the same as R^{1e′}.

In various aspects, R^{1a} is different than R^{1a′}. In a further aspect, R^{1b} is different than R^{1b′}. In a still further aspect, R^{1c} is different than R^{1c′}. In yet a further aspect, R^{1d} different than R^{1d′}. In an even further aspect, R^{1e} is different than R^{1e′}.

In various aspects, each of R^{1a}, R^{1b}, R^{1c}, R^{1d}, and R^{1e} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each of R^{1a}, R^{1b}, R^{1c}, R^{1d}, and R^{1e} is independently selected from hydrogen, —F, —Cl, —NH₂, —CN, —OH, —NO₂, methyl, ethyl, n-propyl, i-propyl, ethenyl, propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a further aspect, each of R^{1a}, R^{1b}, R^{1c}, R^{1d}, and R^{1e} is independently selected from hydrogen, —F, —Cl, —NH₂, —CN, —OH, —NO₂, methyl, ethyl, ethenyl, —CH₂F, —CH₂Cl, —CH₂CH₂F, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —NHCH₃, —NHCH₂CH₃, —N(CH₃)₂, —N(CH₂CH₃)₂, —N(CH₃)(CH₂CH₃), —CH₂NH₂, and —CH₂CH₂NH₂. In a still further aspect, each of R^{1a}, R^{1b}, R^{1c}, R^{1d}, and R^{1e} is independently selected from hydrogen, —F, —Cl, —NH₂, —CN, —OH, —NO₂, methyl, —CH₂F, —CH₂Cl, —CH₂CN, —CH₂OH, —OCF₃, —OCH₂CF₃, —OCH₃, —NHCH₃, —N(CH₃)₂, and —CH₂NH₂.

In various aspects, each of R^{1a}, R^{1b}, R^{1c}, R^{1d}, and R^{1e} is independently selected from hydrogen, halogen, —CN, —NH₂, —OH, —NO₂, C1-C4 alkyl, and C2-C4 alkenyl. Thus, in various further aspects, each of R^{1a}, R^{1b}, R^{1c}, R^{1d}, and R^{1e} is independently selected from hydrogen, —F, —Cl, —NH₂, —CN, —OH, —NO₂, methyl, ethyl, n-propyl, i-propyl, ethenyl, propenyl, and isopropenyl. In a further aspect, each of R^{1a}, R^{1b}, R^{1c}, R^{1d}, and R^{1e} is independently selected from hydrogen, —F, —Cl, —NH₂, —CN, —OH, —NO₂, methyl, ethyl, and ethenyl. In a still further aspect, each of R^{1a}, R^{1b}, R^{1c}, R^{1d}, and R^{1e} is independently selected from hydrogen, —F, —Cl, —NH₂, —CN, —OH, —NO₂, and methyl.

In various aspects, each of R^{1a}, R^{1b}, R^{1c}, R^{1d}, and R^{1e} is independently selected from hydrogen, C1-C4 alkyl, and C2-C4 alkenyl. Thus, in various further aspects, each of R^{1a}, R^{1b}, R^{1c}, R^{1d}, and R^{1e} is independently selected from hydrogen, methyl, ethyl, n-propyl, i-propyl, ethenyl, propenyl, and isopropenyl. In a further aspect, each of R^{1a}, R^{1b}, R^{1c}, R^{1d}, and R^{1e} is independently selected from hydrogen, methyl, ethyl, and ethenyl. In a still further aspect, each of R^{1a}, R^{1b}, R^{1c}, R^{1d}, and R^{1e} is independently selected from hydrogen and methyl.

In various aspects, each of R^{1a}, R^{1b}, R^{1c}, R^{1d}, and R^{1e} is independently selected from hydrogen, halogen, —CN, —NH₂, —OH, —NO₂, C1-C4 haloalkyl, and C1-C4 haloalkoxy. Thus, in various further aspects, each of R^{1a}, R^{1b}, R^{1c}, R^{1d}, and R^{1e} is independently selected from hydrogen, —F, —Cl, —NH₂, —CN, —OH, —NO₂, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCF₃, —OCH₂CF₃, —OCH₂CH₂CF₃, and —OCH(CH₃)CF₃. In a further aspect, each of R^{1a}, R^{1b}, R^{1c}, R^{1d}, and R^{1e} is independently selected from hydrogen, —F, —Cl, —NH₂, —CN, —OH, —NO₂, —CH₂F, —CH₂Cl, —CH₂CH₂F, —CH₂CH₂Cl, —OCF₃, and —OCH₂CF₃. In a still further aspect, each of R^{1a}, Rib, R^{1c}, R^{1d}, and R^{1e} is independently selected from hydrogen, —F, —Cl, —NH₂, —CN, —OH, —NO₂, —CH₂F, —CH₂Cl, —OCF₃, and —OCH₂CF₃.

In various aspects, each of R^{1a}, R^{1b}, R^{1c}, R^{1d}, and R^{1e} is independently selected from hydrogen, halogen, C1-C4 haloalkyl, and C1-C4 haloalkoxy. Thus, in various further aspects, each of R^{1a}, R^{1b}, R^{1c}, R^{1d}, and R^{1e} is independently selected from hydrogen, —F, —Cl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCF₃, —OCH₂CF₃, —OCH₂CH₂CF₃, and —OCH(CH₃)CF₃. In a further aspect, each of R^{1a}, R^{1b}, R^{1c}, R^{1d}, and R^{1e} is independently selected from hydrogen, —F, —Cl, —CH₂F, —CH₂Cl, —CH₂CH₂F, —CH₂CH₂Cl, —OCF₃, and —OCH₂CF₃. In a still further aspect, each of R^{1a}, R^{1b}, R^{1c}, R^{1d}, and R^{1e} is independently selected from hydrogen, —F, —Cl, —CH₂F, —CH₂Cl, —OCF₃, and —OCH₂CF₃.

In various aspects, each of R^{1a}, R^{1b}, R^{1c}, R^{1d}, and R^{1e} is independently selected from hydrogen, halogen, —CN, —NH₂, —OH, —NO₂, and C1-C4 cyanoalkyl. Thus, in various further aspects, each of R^{1a}, R^{1b}, R^{1c}, R^{1d}, and R^{1e} is independently selected from hydrogen, —F, —Cl, —NH₂, —CN, —OH, —NO₂, —CH₂CN, —CH₂CH₂CN, —CH₂CH₂CH₂CN, and —CH(CH₃)CH₂CN. In a further aspect, each of R^{1a}, R^{1b}, R^{1c}, R^{1d}, and R^{1e} is independently selected from hydrogen, —F, —Cl, —NH₂, —CN, —OH, —NO₂, —CH₂CN, and —CH₂CH₂CN. In a still further aspect, each of R^{1a}, R^{1b}, R^{1c}, R^{1d}, and R^{1e} is independently selected from hydrogen, —F, —Cl, —NH₂, —CN, —OH, —NO₂, and —CH₂CN.

In various aspects, each of R^{1a}, R^{1b}, R^{1c}, R^{1d}, and R^{1e} is independently selected from hydrogen and C1-C4 cyanoalkyl. Thus, in various further aspects, each of R^{1a}, R^{1b}, R^{1c}, R^{1d}, and R^{1e} is independently selected from hydrogen, —CH₂CN, —CH₂CH₂CN, —CH₂CH₂CH₂CN, and —CH(CH₃)CH₂CN. In a further aspect, each of R^{1a}, R^{1b}, R^{1c}, R^{1d}, and R^{1e} is independently selected from hydrogen, —CH₂CN, and —CH₂CH₂CN. In a still further aspect, each of R^{1a}, R^{1b}, R^{1c}, R^{1d}, and R^{1e} is independently selected from hydrogen and —CH₂CN.

In various aspects, each of R^{1a}, R^{1b}, R^{1c}, R^{1d}, and R^{1e} is independently selected from hydrogen, halogen, —CN, —NH₂, —OH, —NO₂, C₁-C₄ hydroxyalkyl, and C₁-C₄ alkoxy. Thus, in various further aspects, each of R^{1a}, R^{1b}, R^{1c}, R^{1d}, and R^{1e} is independently selected from hydrogen, —F, —Cl, —NH₂, —CN, —OH, —NO₂, —CH₂OH, —CH₂CH₂OH, —CH₂CH₂CH₂OH, —CH(CH₃)CH₂OH, —OCH₃, —OCH₂CH₃, —OCH₂CH₂CH₃, and —OCH(CH₃)CH₃. In a further aspect, each of R^{1a}, R^{1b}, R^{1c}, R^{1d}, and R^{1e} is independently selected from hydrogen, —F, —Cl, —NH₂, —CN, —OH, —NO₂, —CH₂OH, —CH₂CH₂OH, —OCH₃, and —OCH₂CH₃. In a still further aspect, each of R^{1a}, R^{1b}, R^{1c}, R^{1d}, and R^{1e} is independently selected from hydrogen, —F, —Cl, —NH₂, —CN, —OH, —NO₂, —CH₂OH, and —OCH₃.

In various aspects, each of R^{1a}, R^{1b}, R^{1c}, R^{1d}, and R^{1e} is independently selected from hydrogen, C1-C4 hydroxyalkyl, and C1-C4 alkoxy. Thus, in various further aspects, each of R^{1a}, R^{1b}, R^{1c}, R^{1d}, and R^{1e} is independently selected from hydrogen, —CH₂OH, —CH₂CH₂OH, —CH₂CH₂CH₂OH, —CH(CH₃)CH₂OH, —OCH₃, —OCH₂CH₃, —OCH₂CH₂CH₃, and —OCH(CH₃)CH₃. In a further aspect, each of R^{1a}, R^{1b}, R^{1c}, R^{1d}, and R^{1e} is independently selected from hydrogen, —CH₂OH, —CH₂CH₂OH, —OCH₃, and —OCH₂CH₃. In a still further aspect, each of R^{1a}, R^{1b}, R^{1c}, R^{1d}, and R^{1e} is independently selected from hydrogen, —CH₂OH, and —OCH₃.

In various aspects, each of R^{1a}, R^{1b}, R^{1c}, R^{1d}, and R^{1e} is independently selected from hydrogen, halogen, —CN, —NH₂, —OH, —NO₂, C1-C4 alkylamino, (C1-C4)(C1-C4) dialkylamino, and C1-C4 aminoalkyl. Thus, in various further aspects, each of R^{1a}, R^{1b}, R^{1c}, R^{1d}, and R^{1e} is independently selected from hydrogen, —F, —Cl, —NH₂, —CN, —OH, —NO₂, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a further aspect, each of R^{1a}, R^{1b}, R^{1c}, R^{1d}, and R^{1e} is independently selected from hydrogen, —F, —Cl, —NH₂, —CN, —OH, —NO₂, —NHCH₃, —NHCH₂CH₃, —N(CH₃)₂, —N(CH₂CH₃)₂, —N(CH₃)(CH₂CH₃), —CH₂NH₂, and —CH₂CH₂NH₂. In a still further aspect, each of R^{1a}, R^{1b}, R^{1c}, R^{1d}, and R^{1e} is independently selected from hydrogen, —F, —Cl, —NH₂, —CN, —OH, —NO₂, —NHCH₃, —N(CH₃)₂, and —CH₂NH₂.

In various aspects, each of R^{1a}, R^{1b}, R^{1c}, R^{1d}, and R^{1e} is independently selected from hydrogen, C1-C4 alkylamino, (C1-C4)(C1-C4) dialkylamino, and C1-C4 aminoalkyl. Thus, in various further aspects, each of R^{1a}, R^{1b}, R^{1c}, R^{1d}, and R^{1e} is independently selected from hydrogen, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a further aspect, each of R^{1a}, R^{1b}, R^{1c}, R^{1d}, and R^{1e} is independently selected from hydrogen, —NHCH₃, —NHCH₂CH₃, —N(CH₃)₂, —N(CH₂CH₃)₂, —N(CH₃)(CH₂CH₃), —CH₂NH₂, and —CH₂CH₂NH₂. In a still further aspect, each of R^{1a}, R^{1b}, R^{1c}, R^{1d}, and R^{1e} is independently selected from hydrogen, —NHCH₃, —N(CH₃)₂, and —CH₂NH₂.

In various aspects, each of R^{1a}, R^{1b}, R^{1c}, R^{1d}, and R^{1e} is hydrogen.

In various aspects, each of R^{1a′}, R^{1b′}, R^{1c′}, R^{1d′}, and R^{1e′} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl. In a further aspect, each of R^{1a′}, R^{1b′}, R^{1c′}, R^{1d′}, and R^{1e′} is independently selected from hydrogen, —F, —Cl, —NH₂, —CN, —OH, —NO₂, methyl, ethyl, n-propyl, i-propyl, ethenyl, propenyl, isopropenyl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —CH₂CN, —CH₂CH₂CN, —CH₂CH₂CH₂CN, —CH(CH₃)CH₂CN, —CH₂OH, —CH₂CH₂OH, —CH₂CH₂CH₂OH, —CH(CH₃)CH₂OH, —OCF₃, —OCH₂CF₃, —OCH₂CH₂CF₃, —OCH(CH₃)CF₃, —OCH₃, —OCH₂CH₃, —OCH₂CH₂CH₃, —OCH(CH₃)CH₃, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a further aspect, each of R^{1a′}, R^{1b′}, R^{1c′}, R^{1d′}, and R^{1e′} is independently selected from hydrogen, —F, —Cl, —NH₂, —CN, —OH, —NO₂, methyl, ethyl, ethenyl, —CH₂F, —CH₂Cl, —CH₂CH₂F, —CH₂CH₂Cl, —CH₂CN, —CH₂CH₂CN, —CH₂OH, —CH₂CH₂OH, —OCF₃, —OCH₂CF₃, —OCH₃, —OCH₂CH₃, —NHCH₃, —NHCH₂CH₃, —N(CH₃)₂, —N(CH₂CH₃)₂, —N(CH₃)(CH₂CH₃), —CH₂NH₂, and —CH₂CH₂NH₂. In a still further aspect, each of R^{1a′}, R^{1b′}, R^{1c′}, R^{1d′}, and R^{1e′} is independently selected from hydrogen, —F, —Cl, —NH₂, —CN, —OH, —NO₂, methyl, —CH₂F, —CH₂Cl, —CH₂CN, —CH₂OH, —OCF₃, —OCH₂CF₃, —OCH₃, —NHCH₃, —N(CH₃)₂, and —CH₂NH₂.

In various aspects, each of R^{1a′}, R^{1b′}, R^{1c′}, R^{1d′}, and R^{1e′} is independently selected from hydrogen, halogen, —CN, —NH₂, —OH, —NO₂, C1-C4 alkyl, and C2-C4 alkenyl. Thus, in various further aspects, each of R^{1a′}, R^{1b′}, R^{1c′}, R^{1d′}, and R^{1e′} is independently selected from hydrogen, —F, —Cl, —NH₂, —CN, —OH, —NO₂, methyl, ethyl, n-propyl, i-propyl, ethenyl, propenyl, and isopropenyl. In a further aspect, each of R^{1a′}, R^{1b′}, R^{1c′}, R^{1d′}, and R^{1e′} is independently selected from hydrogen, —F, —Cl, —NH₂, —CN, —OH, —NO₂, methyl, ethyl, and ethenyl. In a still further aspect, each of R^{1a′}, R^{1b′}, R^{1c′}, R^{1d′}, and R^{1e′} is independently selected from hydrogen, —F, —Cl, —NH₂, —CN, —OH, —NO₂, and methyl.

In various aspects, each of R^{1a′}, R^{1b′}, R^{1c′}, R^{1d′}, and R^{1e′} is independently selected from hydrogen, C1-C4 alkyl, and C2-C4 alkenyl. Thus, in various further aspects, each of R^{1a′}, R^{1b′}, R^{1c′}, R^{1d′}, and R^{1e′} is independently selected from hydrogen, methyl, ethyl, n-propyl, i-propyl, ethenyl, propenyl, and isopropenyl. In a further aspect, each of R^{1a′}, R^{1b′}, R^{1c′}, R^{1d′}, and R^{1e′} is independently selected from hydrogen, methyl, ethyl, and ethenyl. In a still further aspect, each of R^{1a′}, R^{1b′}, R^{1c′}, R^{1d′}, and R^{1e′} is independently selected from hydrogen and methyl.

In various aspects, each of R^{1a′}, R^{1b′}, R^{1c′}, R^{1d′}, and R^{1e′} is s independently selected from hydrogen, halogen, —CN, —NH₂, —OH, —NO₂, C1-C4 haloalkyl, and C1-C4 haloalkoxy. Thus, in various further aspects, each of R^{1a′}, R^{1b′}, R^{1c′}, R^{1d′}, and R^{1e′} is independently selected from hydrogen, —F, —Cl, —NH₂, —CN, —OH, —NO₂, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCF₃, —OCH₂CF₃, —OCH₂CH₂CF₃, and —OCH(CH₃)CF₃. In a further aspect, each of R^{1a′}, R^{1b′}, R^{1c′}, R^{1d′}, and R^{1e′} is independently selected from hydrogen, —F, —Cl, —NH₂, —CN, —OH, —NO₂, —CH₂F, —CH₂Cl, —CH₂CH₂F, —CH₂CH₂Cl, —OCF₃, and —OCH₂CF₃. In a still further aspect, each of R^{1a′}, R^{1b′}, R^{1c′}, R^{1d′}, and R^{1e′} is independently selected from hydrogen, —F, —Cl, —NH₂, —CN, —OH, —NO₂, —CH₂F, —CH₂Cl, —OCF₃, and —OCH₂CF₃.

In various aspects, each of R^{1a′}, R^{1b′}, R^{1c′}, R^{1d′}, and R^{1e′} is independently selected from hydrogen, halogen, C1-C4 haloalkyl, and C1-C4 haloalkoxy. Thus, in various further aspects, each of R^{1a′}, R^{1b′}, R^{1c′}, R^{1d′}, and R^{1e′} is independently selected from hydrogen, —F, —Cl, —CH₂F, —CH₂Cl, —CH₂CH₂F, —CH₂CH₂Cl, —CH₂CH₂CH₂F, —CH₂CH₂CH₂Cl, —CH(CH₃)CH₂F, —CH(CH₃)CH₂Cl, —OCF₃, —OCH₂CF₃, —OCH₂CH₂CF₃, and —OCH(CH₃)CF₃. In a further aspect, each of R^{1a′}, R^{1b′}, R^{1c′}, R^{1d′}, and R^{1e′} is independently selected from hydrogen, —F, —Cl, —CH₂F, —CH₂Cl, —CH₂CH₂F, —CH₂CH₂Cl, —OCF₃, and —OCH₂CF₃. In a still further aspect, each of R^{1a′}, R^{1b′}, R^{1c′}, R^{1d}, and R^{1e′} is independently selected from hydrogen, —F, —Cl, —CH₂F, —CH₂Cl, —OCF₃, and —OCH₂CF₃.

In various aspects, each of R^{1a′}, R^{1b′}, R^{1c′}, R^{1d′}, and R^{1e′} is independently selected from hydrogen, halogen, —CN, —NH₂, —OH, —NO₂, and C1-C4 cyanoalkyl. Thus, in various further aspects, each of R^{1a′}, R^{1b′}, R^{1c′}, R^{1d′}, and R^{1e′} is independently selected from hydrogen, —F, —Cl, —NH₂, —CN, —OH, —NO₂, —CH₂CN, —CH₂CH₂CN, —CH₂CH₂CH₂CN, and —CH(CH₃)CH₂CN. In a further aspect, each of R^{1a′}, R^{1b′}, R^{1c′}, R^{1d′}, and R^{1e′} is independently selected from hydrogen, —F, —Cl, —NH₂, —CN, —OH, —NO₂, —CH₂CN, and —CH₂CH₂CN. In a still further aspect, each of R^{1a′}, R^{1b′}, R^{1c′}, R^{1d′}, and R^{1e′} is independently selected from hydrogen, —F, —Cl, —NH₂, —CN, —OH, —NO₂, and —CH₂CN.

In various aspects, each of R^{1a′}, R^{1b′}, R^{1c′}, R^{1d′}, and R^{1e′} is independently selected from hydrogen and C1-C4 cyanoalkyl. Thus, in various further aspects, each of R^{1a′}, R^{1b′}, R^{1c′}, R^{1d′}, and R^{1e′} is independently selected from hydrogen, —CH₂CN, —CH₂CH₂CN, —CH₂CH₂CH₂CN, and —CH(CH₃)CH₂CN. In a further aspect, each of R^{1a′}, R^{1b′}, R^{1c′}, R^{1d′}, and R^{1e′} is independently selected from hydrogen, —CH₂CN, and —CH₂CH₂CN. In a still further aspect, each of R^{1a′}, R^{1b′}, R^{1c′}, R^{1d′}, and R^{1e′} is independently selected from hydrogen and —CH₂CN.

In various aspects, each of R^{1a′}, R^{1b′}, R^{1c′}, R^{1d′}, and R^{1e′} is independently selected from hydrogen, halogen, —CN, —NH₂, —OH, —NO₂, C1-C4 hydroxyalkyl, and C1-C4 alkoxy. Thus, in various further aspects, each of R^{1a′}, R^{1b′}, R^{1c′}, R^{1d′}, and R^{1e′} is independently selected from hydrogen, —F, —Cl, —NH₂, —CN, —OH, —NO₂, —CH₂OH, —CH₂CH₂OH, —CH₂CH₂CH₂OH, —CH(CH₃)CH₂OH, —OCH₃, —OCH₂CH₃, —OCH₂CH₂CH₃, and —OCH(CH₃)CH₃. In a further aspect, each of R^{1a′}, R^{1b′}, R^{1c′}, R^{1d′}, and R^{1e′} is independently selected from hydrogen, —F, —Cl, —NH₂, —CN, —OH, —NO₂, —CH₂OH, —CH₂CH₂OH, —OCH₃, and —OCH₂CH₃. In a still further aspect, each of R^{1a′}, R^{1b′}, R^{1c′}, R^{1d′}, and R^{1e′} is independently selected from hydrogen, —F, —Cl, —NH₂, —CN, —OH, —NO₂, —CH₂OH, and —OCH₃.

In various aspects, each of R^{1a′}, R^{1b′}, R^{1c′}, R^{1d′}, and R^{1e′} is independently selected from hydrogen, C1-C4 hydroxyalkyl, and C1-C4 alkoxy. Thus, in various further aspects, each of R^{1a′}, R^{1b′}, R^{1c′}, R^{1d′}, and R^{1e′} is independently selected from hydrogen, —CH₂OH, —CH₂CH₂OH, —CH₂CH₂CH₂OH, —CH(CH₃)CH₂OH, —OCH₃, —OCH₂CH₃, —OCH₂CH₂CH₃, and —OCH(CH₃)CH₃. In a further aspect, each of R^{1a′}, R^{1b′}, R^{1c′}, R^{1d′}, and R^{1e′} is independently selected from hydrogen, —CH₂OH, —CH₂CH₂OH, —OCH₃, and —OCH₂CH₃. In a still further aspect, each of R^{1a′}, R^{1b′}, R^{1c′}, R^{1d′}, and R^{1e′} is independently selected from hydrogen, —CH₂OH, and —OCH₃.

In various aspects, each of R^{1a′}, R^{1b′}, R^{1c′}, R^{1d′}, and R^{1e′} is independently selected from hydrogen, halogen, —CN, —NH₂, —OH, —NO₂, C1-C4 alkylamino, (C1-C4)(C1-C4) dialkylamino, and C1-C4 aminoalkyl. Thus, in various further aspects, each of R^{1a′}, R^{1b′}, R^{1c′}, R^{1d′}, and R^{1e′} is independently selected from hydrogen, —F, —Cl, —NH₂, —CN, —OH, —NO₂, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a further aspect, each of R^{1a′} R^{1b′}, R^{1e′}, R^{1d′}, and R^{1e′} is independently selected from hydrogen, —F, —Cl, —NH₂, —CN, —OH, —NO₂, —NHCH₃, —NHCH₂CH₃, —N(CH₃)₂, —N(CH₂CH₃)₂, —N(CH₃)(CH₂CH₃), —CH₂NH₂, and —CH₂CH₂NH₂. In a still further aspect, each of R^{1a′}, R^{1b′}, R^{1c′}, R^{1d′}, and R^{1e′} is independently selected from hydrogen, —F, —Cl, —NH₂, —CN, —OH, —NO₂, —NHCH₃, —N(CH₃)₂, and —CH₂NH₂.

In various aspects, each of R^{1a′}, R^{1b′}, R^{1c′}, R^{1d′}, and R^{1e′} is independently selected from hydrogen, C1-C4 alkylamino, (C1-C4)(C1-C4) dialkylamino, and C1-C4 aminoalkyl. Thus, in various further aspects, each of R^{1a′}, R^{1b′}, R^{1c′}, R^{1d′}, and R^{1e′} is independently selected from hydrogen, —NHCH₃, —NHCH₂CH₃, —NHCH₂CH₂CH₃, —NHCH(CH₃)CH₃, —N(CH₃)₂, —N(CH₂CH₃)₂, —N(CH₂CH₂CH₃)₂, —N(CH(CH₃)CH₃)₂, —N(CH₃)(CH₂CH₃), —CH₂NH₂, —CH₂CH₂NH₂, —CH₂CH₂CH₂NH₂, and —CH(CH₃)CH₂NH₂. In a further aspect, each of R^{1a′}, R^{1b′}, R^{1c′}, R^{1d′}, and R^{1e′} is independently selected from hydrogen, —NHCH₃, —NHCH₂CH₃, —N(CH₃)₂, —N(CH₂CH₃)₂, —N(CH₃)(CH₂CH₃), —CH₂NH₂, and —CH₂CH₂NH₂. In a still further aspect, each of R^{1a′}, R^{1b′}, R^{1c′}, R^{1d′}, and R^{1e′} is independently selected from hydrogen, —NHCH₃, —N(CH₃)₂, and —CH₂NH₂.

In various aspects, each of R^{1a′}, R^{1b′}, R^{1c′}, R^{1d′}, and R^{1e′} is hydrogen.

b. R^{2A}, R^{2B}, R^{2A′}, and R^{2B′} Groups

In one aspect, each of R^{2a}, R^{2a′}, R^{2b}, and R^{2b′} is independently selected from hydrogen, halogen, —OH, —NH₂, C1-C4 alkyl, and C2-C4 alkenyl. In a further aspect, each of R^{2a}, R^{2a′}, R^{2b}, and R^{2b′} is hydrogen.

In various aspects, R^{2a} is the same as R^{2a′}. In a further aspect, R^{2b} is the same as R^{2b′}.

In various aspects, R^{2a} is different than R^{2a′}. In a further aspect, R^{2b} is different than R^{2b′}.

In various aspects, each of R^{2a} and R^{2b} is independently selected from hydrogen, halogen, —OH, —NH₂, C1-C4 alkyl, and C2-C4 alkenyl. In a further aspect, each of R^{2a} and R^{2b} is independently selected from hydrogen, —F, —Cl, —NH₂, —OH, methyl, ethyl, n-propyl, i-propyl, ethenyl, propenyl, isopropenyl. In a still further aspect, each of R^{2a} and R^{2b} is independently selected from hydrogen, —F, —Cl, —NH₂, —OH, methyl, ethyl, and ethenyl. In yet a further aspect, each of R^{2a} and R^{2b} is independently selected from hydrogen, —F, —Cl, —NH₂, —OH, and methyl.

In various aspects, each of R^{2a} and R^{2b} is independently selected from hydrogen, halogen, C1-C4 alkyl, and C2-C4 alkenyl. In a further aspect, each of R^{2a} and R^{2b} is independently selected from hydrogen, —F, —Cl, methyl, ethyl, n-propyl, i-propyl, ethenyl, propenyl, isopropenyl. In a still further aspect, each of R^{2a} and R^{2b} is independently selected from hydrogen, —F, —Cl, methyl, ethyl, and ethenyl. In yet a further aspect, each of R^{2a} and R^{2b} is independently selected from hydrogen, —F, —Cl, and methyl.

In various aspects, each of R^{2a} and R^{2b} is independently selected from hydrogen, and C1-C4 alkyl. In a further aspect, each of R^{2a} and R^{2b} is independently selected from hydrogen, methyl, ethyl, n-propyl, and i-propyl. In a still further aspect, each of R^{2a} and R^{2b} is independently selected from hydrogen, methyl, and ethyl. In yet a further aspect, each of R^{2a} and R^{2b} is independently selected from hydrogen and methyl.

In various aspects, each of R^{2a} and R^{2b} is independently selected from hydrogen and C2-C4 alkenyl. In a further aspect, each of R^{2a} and R^{2b} is independently selected from hydrogen, ethenyl, propenyl, isopropenyl. In a still further aspect, each of R^{2a} and R^{2b} is independently selected from hydrogen and ethenyl.

In various aspects, each of R^{2a} and R^{2b} is independently selected from hydrogen and halogen. In a further aspect, each of R^{2a} and R^{2b} is independently selected from hydrogen, —F, —Cl, and —Br. In a still further aspect, each of R^{2a} and R^{2b} is independently selected from hydrogen, —F, and —Cl. In yet a further aspect, each of R^{2a} and R^{2b} is independently selected from hydrogen and —F.

In various aspects, each of R^{2a} and R^{2b} is hydrogen.

In various aspects, each of R^{2a′} and R^{2b′} is independently selected from hydrogen, halogen, —OH, —NH₂, C1-C4 alkyl, and C2-C4 alkenyl. In a further aspect, each of R^{2a′} and R^{2b′} is independently selected from hydrogen, —F, —Cl, —NH₂, —OH, methyl, ethyl, n-propyl, i-propyl, ethenyl, propenyl, isopropenyl. In a still further aspect, each of R^{2a′} and R^{2b′} is independently selected from hydrogen, —F, —Cl, —NH₂, —OH, methyl, ethyl, and ethenyl. In yet a further aspect, each of R^{2a′} and R^{2b′} is independently selected from hydrogen, —F, —Cl, —NH₂, —OH, and methyl.

In various aspects, each of R^{2a′} and R^{2b′} is independently selected from hydrogen, halogen, C1-C4 alkyl, and C2-C4 alkenyl. In a further aspect, each of R^{2a′} and R^{2b′} is independently selected from hydrogen, —F, —Cl, methyl, ethyl, n-propyl, i-propyl, ethenyl, propenyl, isopropenyl. In a still further aspect, each of R^{2a′} and R^{2b′} is independently selected from hydrogen, —F, —Cl, methyl, ethyl, and ethenyl. In yet a further aspect, each of R^{2a′} and R^{2b′} is independently selected from hydrogen, —F, —Cl, and methyl.

In various aspects, each of R^{2a′} and R^{2b′} is independently selected from hydrogen, and C1-C4 alkyl. In a further aspect, each of R^{2a′} and R^{2b′} is independently selected from hydrogen, methyl, ethyl, n-propyl, and i-propyl. In a still further aspect, each of R^{2a′} and R^{2b′} is independently selected from hydrogen, methyl, and ethyl. In yet a further aspect, each of R^{2a′} and R^{2b′} is independently selected from hydrogen and methyl.

In various aspects, each of R^{2a′} and R^{2b′} is independently selected from hydrogen and C2-C4 alkenyl. In a further aspect, each of R^{2a′} and R^{2b′} is independently selected from hydrogen, ethenyl, propenyl, isopropenyl. In a still further aspect, each of R^{2a′} and R^{2b′} is independently selected from hydrogen and ethenyl.

In various aspects, each of R^{2a′} and R^{2b′} is independently selected from hydrogen and halogen. In a further aspect, each of R^{2a′} and R^{2b′} is independently selected from hydrogen, —F, —Cl, and —Br. In a still further aspect, each of R^{2a′} and R^{2b′} is independently selected from hydrogen, —F, and —Cl. In yet a further aspect, each of R^{2a′} and R^{2b′} is independently selected from hydrogen and —F.

In various aspects, each of R^{2a′} and R^{2b′} is hydrogen.

c. R³ and R^{3′} Groups

In one aspect, each of R³ and R^{3′} is independently selected from hydrogen and C1-C4 alkyl. In a further aspect, each of R³ and R^{3′} is hydrogen.

In various aspects, R³ is the same as R^{3′}.

In various aspects, R³ is different than R^{3′}.

In various aspects, R³ is selected from hydrogen and C1-C4 alkyl. In a further aspect, R³ is selected from hydrogen, methyl, ethyl, n-propyl, and isopropyl. In yet a further aspect, R³ is selected from hydrogen, methyl, and ethyl. In an even further aspect, R³ is selected from hydrogen and methyl.

In various aspects, R³ is C1-C4 alkyl. In a further aspect, R³ is selected from methyl, ethyl, n-propyl, and isopropyl. In yet a further aspect, R³ is selected from methyl and ethyl. In an even further aspect, R³ is methyl.

In various aspects, R³ is hydrogen.

In various aspects, R^{3′} is selected from hydrogen and C1-C4 alkyl. In a further aspect, R^{3′} is selected from hydrogen, methyl, ethyl, n-propyl, and isopropyl. In yet a further aspect, R^{3′} is selected from hydrogen, methyl, and ethyl. In an even further aspect, R^{3′} is selected from hydrogen and methyl.

In various aspects, R^{3′} is C1-C4 alkyl. In a further aspect, R^{3′} is selected from methyl, ethyl, n-propyl, and isopropyl. In yet a further aspect, R^{3′} is selected from methyl and ethyl. In an even further aspect, R^{3′} is methyl.

In various aspects, R^{3′} is hydrogen.

### 2. Prophetic Compound Examples

The following compound examples are prophetic, and can be prepared using the synthesis methods described herein above and other general methods as needed as would be known to one skilled in the art. It is anticipated that the prophetic compounds would be useful in treating psychological disorders, as described elsewhere here, and that such utility can be determined by methods known to one of ordinary skill in the art.

In one aspect, a compound can be selected from:

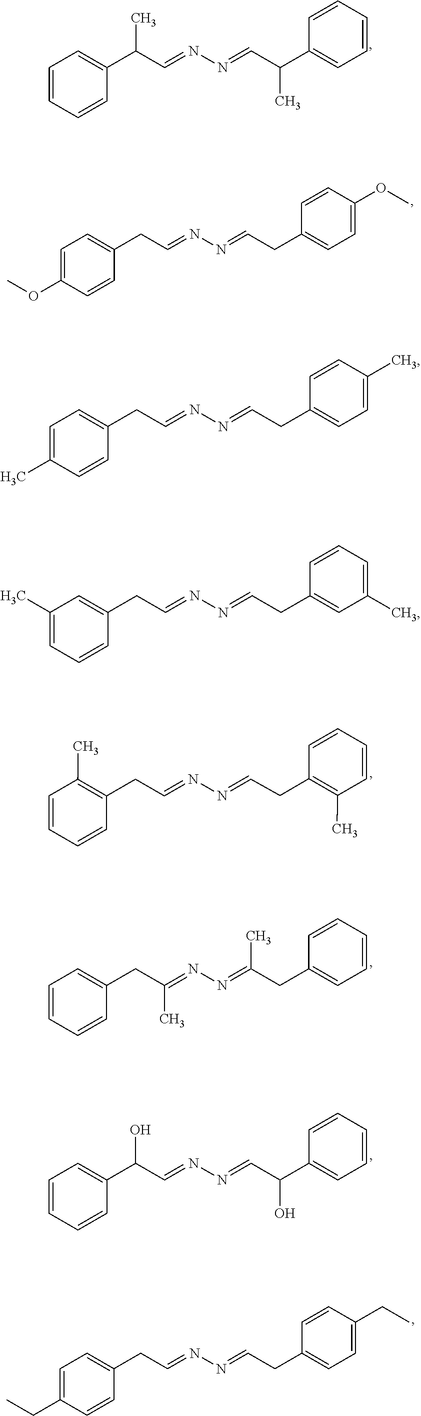

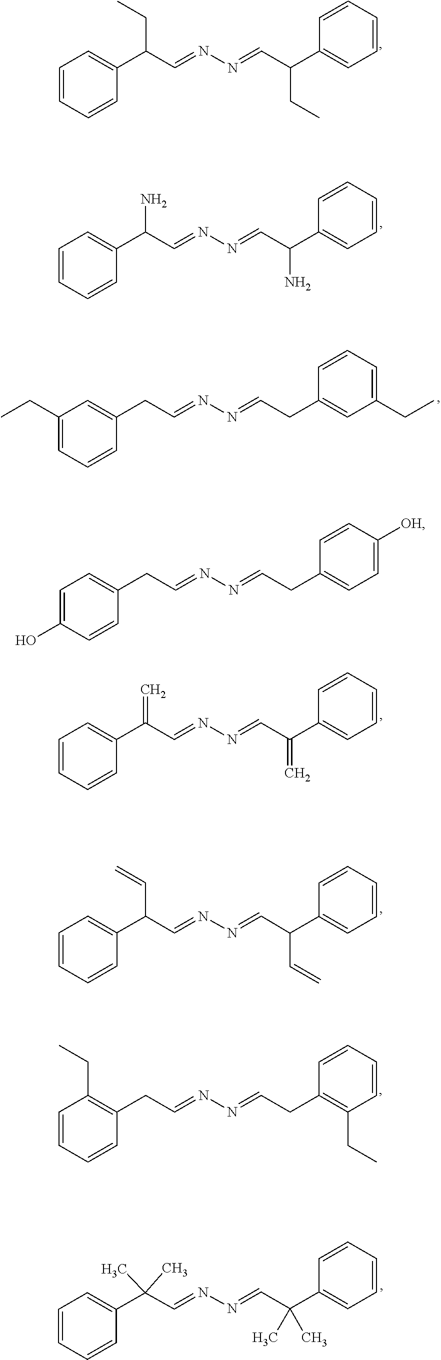

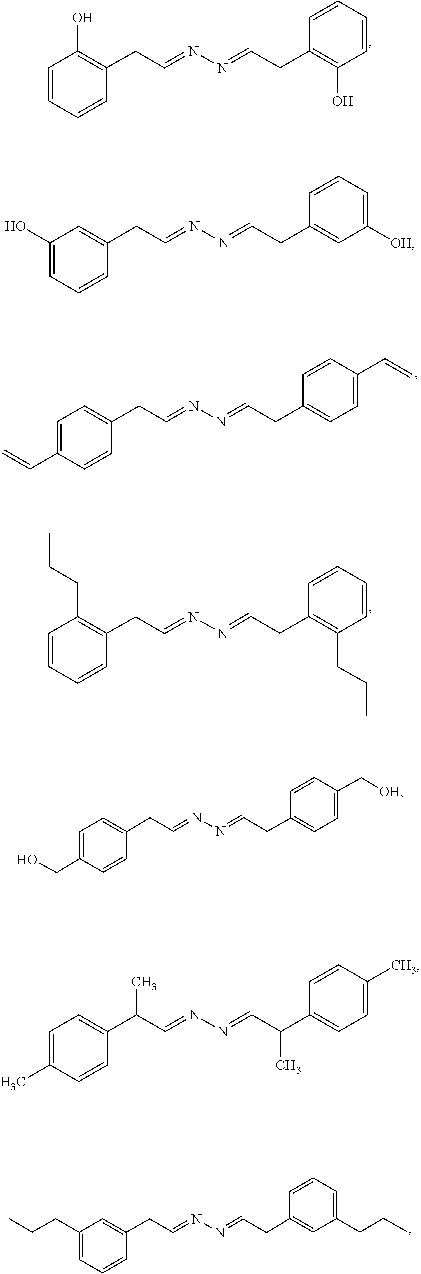

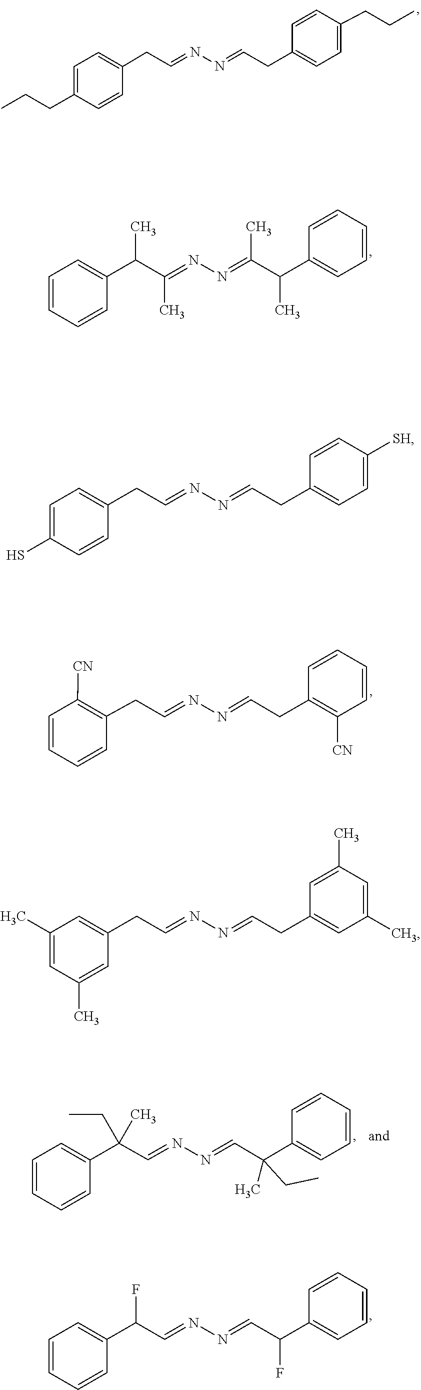

or a pharmaceutically acceptable salt thereof.

In one aspect, a compound can be selected from:

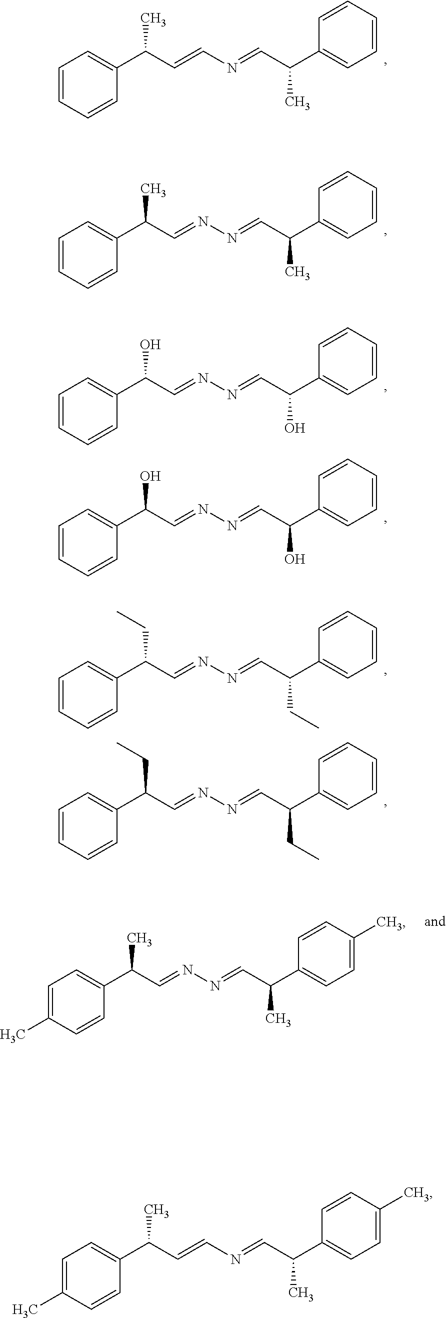

or a pharmaceutically acceptable salt thereof.

### C. Pharmaceutical Compositions

In one aspect, disclosed are pharmaceutical compositions comprising a therapeutically effective amount of a compound having a structure represented by a formula:

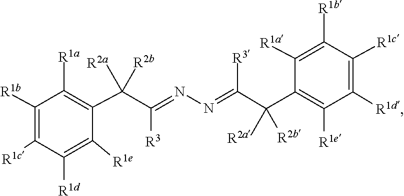

wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a}, R^{2a′}, R^{2b}, and R^{2b′} is independently selected from hydrogen, halogen, —OH, —NH₂, C1-C4 alkyl, and C2-C4 alkenyl; and wherein each of R³ and R^{3′} is independently selected from hydrogen and C1-C4 alkyl, or a pharmaceutically acceptable salt thereof, and a pharmaceutically acceptable carrier. In a further aspect, the compound is laterally symmetric.

Also disclosed are disclosed are pharmaceutical compositions comprising a therapeutically effective amount of a compound having a structure:

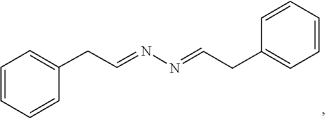 [SMILES: C(/Cc1ccccc1)=N\N=C\Cc1ccccc1]

or a pharmaceutically acceptable salt thereof, and a pharmaceutically acceptable carrier.

Also disclosed are pharmaceutical compositions comprising a therapeutically effective amount of a compound having a structure represented by a formula:

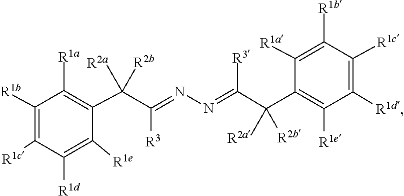

wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a}, R^{2a′}, R^{2b}, and R^{2b′} is independently selected from hydrogen, halogen, —OH, —NH₂, C1-C4 alkyl, and C2-C4 alkenyl; and wherein each of R³ and R^{3′} is independently selected from hydrogen and C1-C4 alkyl, or a pharmaceutically acceptable salt thereof, and an antidepressant, a biocompatible polymer, and a pharmaceutically acceptable carrier.

Also disclosed are pharmaceutical compositions comprising a therapeutically effective amount of a compound having a structure:

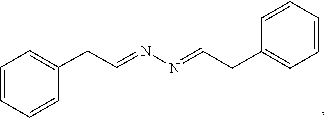 [SMILES: C(/Cc1ccccc1)=N\N=C\Cc1ccccc1]

or a pharmaceutically acceptable salt thereof and an antidepressant, a biocompatible polymer, and a pharmaceutically acceptable carrier.

In various aspects, the composition is formulated as a single oral dosage form.

In various aspects, the composition further comprises an effective amount of an antidepressant, for example, a serotonergic antidepressant. Examples of antidepressants include, but are not limited to, selective serotonin reuptake inhibitors (SSRIs) (e.g., citalopram, dapoxetine, escitalopram, fluoxetine, fluvoxamine, paroxetine, sertraline, vortioxetine), serotonin-norepinephrine reuptake inhibitors (SNRIs) (e.g., desvenlafaxine, duloxetine, levomilnacipran, venlafaxine), tricyclic antidepressants (TCAs) (e.g., amitriptyline, amoxapine, desipramine, doxepin, imipramine, nortriptyline, protriptyline, and trimipramine), and 5HT_{1A} receptor agonists (e.g., buspirone, trazodone, nefazodone, vortioxetine, flibanserin, etappirone, lesopitron, alnespirone, repinotan, gepirone). In a further aspect, the composition further comprises an effective amount of a 5HT_{1A} receptor agonist. In a still further aspect, the 5HT_{1A} receptor agonist is buspirone.

In various aspects, the compound and the antidepressant are each present in individually therapeutically effective amounts. In a further aspect, the compound and the antidepressant are together present in a therapeutically effective amount.

In various aspects, the composition further comprises a biocompatible polymer. Examples of biocompatible polymers include, but are not limited to, polylactide-polyglycolide, poly(orthoesters), and poly(anhydrides). In a further aspect, the biocompatible polymer is biodegradable. In a still further aspect, the biocompatible polymer is a natural biocompatible polymer. In yet a further aspect, the biocompatible polymer is a synthetic biocompatible polymer.

In various aspects, the composition is formulated as an oral dosage form. In a further aspect, the composition is formulated for intranasal administration. In a still further aspect, the composition is formulated for transdermal or intradermal administration.

The pharmaceutical compositions comprise the compounds in a pharmaceutically acceptable carrier. A pharmaceutically acceptable carrier refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. The compounds can be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

In various aspects, the disclosed pharmaceutical compositions comprise the disclosed compounds (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form should be sterile and should be effectively fluid for easy syringability. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In various aspects, the pharmaceutical compositions of this invention can include a pharmaceutically acceptable carrier and a compound or a pharmaceutically acceptable salt of the compounds of the invention. The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

Pharmaceutically acceptable salts of the compounds are conventional acid-addition salts or base-addition salts that retain the biological effectiveness and properties of the compounds and are formed from suitable non-toxic organic or inorganic acids or organic or inorganic bases. Exemplary acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Example base-addition salts include those derived from ammonium, potassium, sodium and, quaternary ammonium hydroxides, such as for example, tetramethylammonium hydroxide. Chemical modification of a pharmaceutical compound into a salt is a known technique to obtain improved physical and chemical stability, hygroscopicity, flowability, and solubility of compounds. See, e.g., H. Ansel et. al., Pharmaceutical Dosage Forms and Drug Delivery Systems (6th Ed. 1995) at pp. 196 and 1456-1457.

In some aspects, to prolong the effect of a compound utilized herein, it may be desirable to slow the absorption of the compound (e.g., via preparation of a controlled-release formulation). This may be accomplished, for example, via a liquid suspension of crystalline or amorphous material with poor water solubility. The rate of absorption of the compound then depends upon its rate of dissolution that, in turn, may depend upon crystal size and crystalline form. Alternatively, delayed absorption of a compound can be accomplished by dissolving or suspending the compound in an oil vehicle. Controlled-release formulations can also be prepared by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Alternatively, controlled-release formulations can be prepared by entrapping the compound in liposomes or microemulsions that are compatible with body tissues.

In some aspects, the disclosed controlled-release dosage form is formulation such that one or more of the dissolution, release, delivery, and/or pharmacokinetic properties disclosed herein are satisfied. Thus, for example, Cmax (i.e., peak drug concentrations in blood or plasma after dosing) can be influenced by drug dose (e.g., a higher dose usually produces higher Cmax values), route of administration (e.g., higher Cmax values may occur after IV bolus dosing compared with oral dosing), and the type of formulation (e.g., a lower Cmax may occur after dosing with an controlled release oral formulation compared with an immediate release co formulation). The controlled release formulation reduces the Cmax for a given daily dose, by making the drug available longer but at a lower concentration. Other drug characteristics such as solubility, permeability, ways in which it is absorbed into the body, metabolism, and metabolic products, etc., can also influence Cmax, which means that although certain projections may be made based on the factors mentioned above, the actual behavior observed is difficult to predict without significant experimentation in humans and may be unexpected.

In some aspects, the disclosed controlled-release formulations are formulated in accordance with routine procedures as a composition adapted for oral administration to human subjects. Compositions for oral delivery can take a variety of forms including, but not limited to, tablets, lozenges, aqueous or oil suspensions, granules, powders, emulsions, capsules, syrups, or elixirs. Orally administered compositions can also contain one or more sweetening agents such as fructose, aspartame, or saccharin; flavoring agents such as peppermint, oil of wintergreen, or cherry; coloring agents; and/or preserving agents, to provide a pharmaceutically palatable preparation. Moreover, where in tablet or pill form, the composition can be coated to delay disintegration and absorption in the gastrointestinal tract, thereby providing a sustained action over an extended period of time. Selectively permeable membranes surrounding an osmotically active compound are also suitable for oral administration. In these latter forms, fluid from the environment surrounding the capsule is imbibed by the driving compound, which swells to displace the agent or agent formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In some aspects, the excipients are of pharmaceutical grade.

In some aspects, the controlled-release formulations can be administered by controlled-release means or by delivery devices that are well known to those of ordinary skill in the art. Examples include, but are not limited to, those described in U.S. Pat. Nos. 3,845,770; 3,916,899, 3,536,809; 3,598,123; 4,008,719; 5,674,533; 5,059,595; 5,591,767; 5,120,548; 5,073,543; 5,639,476; 5,354,556; and 5,733,556. Such dosage forms can be useful for providing controlled- or sustained-release of the compositions disclosed herein using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Thus, in some aspects, disclosed herein are single unit dosage forms suitable for oral administration such as, but not limited to, tablets, capsules, gelcaps, and caplets that are adapted for controlled- or sustained-release.

### D. Methods of Making a Compound

The compounds of this invention can be prepared by employing reactions as shown in the following schemes, in addition to other standard manipulations that are known in the literature, exemplified in the experimental sections or clear to one skilled in the art. For clarity, examples having a single substituent are shown where multiple substituents are allowed under the definitions disclosed herein.

Reactions used to generate the compounds of this invention are prepared by employing reactions as shown in the following Reaction Schemes, as described and exemplified below. In certain specific examples, the disclosed compounds can be prepared by Route I, as described and exemplified below. The following examples are provided so that the invention might be more fully understood, are illustrative only, and should not be construed as limiting.

### 1. Route I

In one aspect, substituted PEH derivatives and substituted azine derivatives can be prepared as shown below.

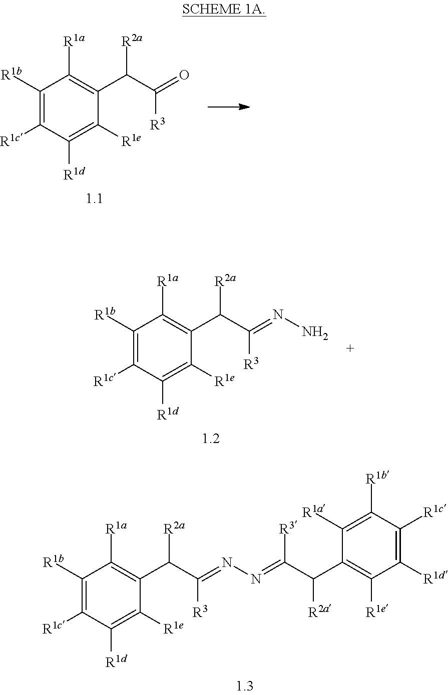

Compounds are represented in generic form, with substituents as noted in compound descriptions elsewhere herein. A more specific example is set forth below.

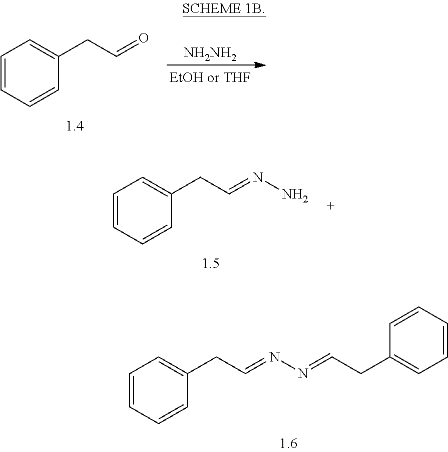

In one aspect, compounds of type 1.5 and type 1.6, and similar compounds, can be prepared according to reaction Scheme 1B above. Thus, compounds of type 1.5 and type 1.6 can be prepared by a nucleophilic addition of hydrazine to an appropriate aldehyde. Appropriate aldehydes are commercially available or prepared by methods known to one skilled in the art. The nucleophilic reaction is carried out in an appropriate solvent, e.g., ethanol or tetrahydrofuran. As can be appreciated by one skilled in the art, the above reaction provides an example of a generalized approach wherein compounds similar in structure to the specific reactants above (compounds similar to compounds of type 1.1), can be substituted in the reaction to provide substituted PEH derivatives and substituted azine derivatives similar to Formulas 1.5 and 1.6.

### E. Methods for Treating a Psychological Disorder

Phenelzine (PLZ) is a potent irreversible, non-selective monoamine oxidase inhibitor (MAOI). It is used clinically for the treatment of a number of psychiatric disorders, including major depression (McGrath, et al. 1986), atypical depression (Paykel, et al. 1982), and social anxiety disorder (Liebowitz, et al. 1988). Interestingly, PLZ is also an inhibitor of γ-aminobutyric acid (GABA) and alanine (ALA), presumably due to its inhibition of GABA transaminase and ALA transaminase, respectively ((Popov and Matthies (1969) J Neurochem 16(3): 899-907; Paslawski, et al. (1995) Prog Brain Res 106: 181-186). It has been suggested that a metabolite of PLZ formed by the action of monoamine oxidase (MAO) is primarily responsible for these beneficial effects, since PLZ-induced increases in GABA and ALA can be abolished by pre-treating the animals with another MAO inhibitor (Popov and Matthies (1969; Todd and Baker (1995) J Affect Disord 35: 125-129; MacKenzie, et al. (2009) “Neurochemical and neuroprotective aspects of phenelzine and its active metabolite 0-phenylethylidenehydrazine,” University of Alberta, Dissertation). This metabolite has since been identified as PEH (MacKenzie (2009)).

Although PEH and PLZ share many pharmacological properties, an important distinction is that PEH is only a weak and transient inhibitor of MAO-A and MAO-B (Paslawski, et al. (2001) Drug Dev Res 54: 35-39; MacKenzie, et al. (2008) Bioorg Med Chem 16(17): 8254-8263). A major drawback to clinical use of PLZ is a potential interaction with tyramine-containing foods such as aged cheeses and meats, overripe fruits and vegetables, and fermented beverages. By irreversibly inhibiting MAO-A in the gut, PLZ prevents the metabolism of dietary tyramine, a sympathomimetic agent, which can then enter the bloodstream and cause unpleasant symptoms ranging from headache to hypertensive crisis. Notably, because PEH is a poor inhibitor of MAO-A, it should not be associated with this adverse effect. Unfortunately, despite the promising therapeutic relevance of PEH, PEH is not used in the clinic due to stability concerns and also difficulties with purification.

The disclosed compound offers improvements in one or both of these areas; namely, improved stability and relatively straightforward purification. In particular, the disclosed compound beneficially behaves as a prodrug that is hydrolyzed at relatively lower pH (e.g., a pH of 9.2 or less, 8.5 or less, 8.0 or less, or 7.5 or less) to form PEH.

Thus, in one aspect, disclosed are methods for treating a psychological disorder in a subject in need thereof, the method comprising administering to the subject an effective amount of a compound having a structure represented by a formula:

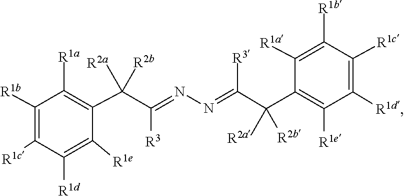

wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a}, R^{2a′}, R^{2b}, and R^{2b′} is independently selected from hydrogen, halogen, —OH, —NH₂, C1-C4 alkyl, and C2-C4 alkenyl; and wherein each of R³ and R^{3′} is independently selected from hydrogen and C1-C4 alkyl, or a pharmaceutically acceptable salt thereof.

In one aspect, disclosed are methods for treating a psychological disorder in a subject in need thereof, the method comprising administering to the subject an effective amount of a compound having a structure:

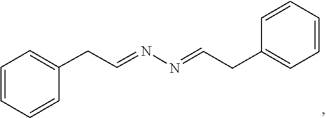 [SMILES: C(/Cc1ccccc1)=N\N=C\Cc1ccccc1]

or a pharmaceutically acceptable salt thereof.

In one aspect, disclosed are methods for treating a psychological disorder in a subject in need thereof, the method comprising administering to the subject an effective amount of a compound having a structure represented by a formula:

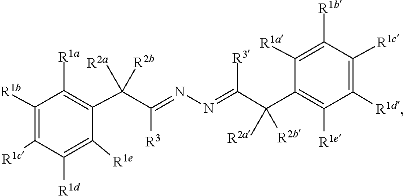

wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a}, R^{2a′}, R^{2b}, and R^{2b′} is independently selected from hydrogen, halogen, —OH, —NH₂, C1-C4 alkyl, and C2-C4 alkenyl; and wherein each of R³ and R^{3′} is independently selected from hydrogen and C1-C4 alkyl, or a pharmaceutically acceptable salt thereof, and an antidepressant, wherein the subject has not responded to at least one adequate antidepressant treatment prior to the administering step.

In one aspect, disclosed are methods for treating a psychological disorder in a subject in need thereof, the method comprising administering to the subject an effective amount of a compound having a structure:

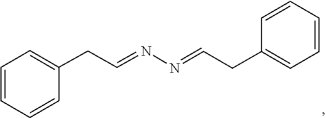 [SMILES: C(/Cc1ccccc1)=N\N=C\Cc1ccccc1]

or a pharmaceutically acceptable salt thereof, and an antidepressant, wherein the subject has not responded to at least one adequate antidepressant treatment prior to the administering step.

In various aspects, the method further comprises administering an effective amount of an antidepressant. Examples of antidepressants include, but are not limited to, selective serotonin reuptake inhibitors (SSRIs) (e.g., citalopram, dapoxetine, escitalopram, fluoxetine, fluvoxamine, paroxetine, sertraline, vortioxetine), serotonin-norepinephrine reuptake inhibitors (SNRIs) (e.g., desvenlafaxine, duloxetine, levomilnacipran, venlafaxine), tricyclic antidepressants (TCAs) (e.g., amitriptyline, amoxapine, desipramine, doxepin, imipramine, nortriptyline, protriptyline, and trimipramine), and 5HT_{1A} receptor agonists (e.g., buspirone, trazodone, nefazodone, vortioxetine, flibanserin, etappirone, lesopitron, alnespirone, repinotan, gepirone). In a further aspect, the method further comprises administering an effective amount of a 5HT_{1A} receptor agonist. In a still further aspect, the 5HT_{1A} receptor agonist is buspirone.

In various aspects, the compound and the antidepressant are administered sequentially. For example, in a further aspect, the compound is administered prior to administration of the antidepressant. In a still further aspect, the compound is administered subsequent to administration of the compound. In yet a further aspect, the compound and the antidepressant are administered simultaneously.

To treat or control the disorder, the compounds and pharmaceutical compositions comprising the compounds are administered to a subject in need thereof. The term does not denote a particular age or sex. Thus, adult and newborn subjects, as well as fetuses, whether male or female, are intended to be covered. The subject is preferably a mammal, such as a human. Prior to administering the compounds or compositions, the subject can be diagnosed with a need for treatment of a psychiatric disorder, such as depression.

Thus, in various aspects, the subject is a mammal. In a further aspect, the mammal is a human.

In various aspects, the subject has been diagnosed as having the psychological disorder prior to the administering step. In a further aspect, the subject has not been diagnosed as having a seizure disorder (e.g., focal seizures, generalized seizures), a developmental disorder (e.g., attention deficit hyperactivity disorder (ADHD), autism spectrum disorder, cerebral palsy), a neurological disorder (e.g., acute spinal cord injury, Alzheimer's disease, amyotrophic lateral sclerosis (ALS), ataxia, Bell's palsy, cerebral aneurysm, Guillain-Barre syndrome, hydrocephalus, meningitis, Parkinson's disease), a behavioral disorder (e.g., ADHD, oppositional defiant disorder (ODD), autism spectrum disorder (ASD), anxiety disorder, depression), or addiction (e.g., substance abuse, substance dependence, chemical dependence) prior to the administering step. In a still further aspect, the subject has not been diagnosed as having a disorder associated with depression (i.e., a disorder that is a comorbidity with depression) such as, for example, addiction, a substance use disorder (e.g., alcohol use disorder, nicotine use disorder), a personality disorder (e.g., borderline personality disorder), post-traumatic stress disorder, and obsessive-compulsive disorder.

In various aspects, the method further comprises identifying a subject in need of treatment of a psychological disorder.

The compounds or compositions can be administered to the subject according to any method. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration and parenteral administration, including injectable such as intravenous administration, intra-arterial administration, intramuscular administration, and subcutaneous administration. Administration can be continuous or intermittent. A preparation can be administered therapeutically; that is, administered to treat an existing disease or condition. A preparation can also be administered prophylactically; that is, administered for prevention of an infection or condition, such as a psychiatric disorder.

In various aspects, the effective amount is a prophylactically effective amount. In a further aspect, the effective amount is a therapeutically effective amount.

The therapeutically effective amount or dosage of the compound can vary within wide limits. Such a dosage is adjusted to the individual requirements in each particular case including the specific compound(s) being administered, the route of administration, the condition being treated, as well as the patient being treated. In general, in the case of oral or parenteral administration to adult humans weighing approximately 70 kg or more, a daily dosage of about 10 mg to about 1000 mg, preferably from about 20 mg to about 800 mg, should be appropriate, although the upper limit may be exceeded. The daily dosage can be administered as a single dose or in divided doses, or for parenteral administration, as a continuous infusion. Single dose compositions can contain such amounts or submultiples thereof of the compound or composition to make up the daily dose. The dosage can be adjusted by the individual physician in the event of any contraindications. Dosage can vary, and can be administered in one or more dose administrations daily, for one or several days.

In various aspects, the compound is formulated as an oral dosage form. In a further aspect, the compound is administered as a single dosage form. In yet a further aspect, the compound is orally administered as a single dosage form. In an even further aspect, the compound is intranasally administered as a single dosage form. In a still further aspect, the compound is topically administered as a single dosage form.

In some aspects, the compound is administered at a dose of from about 0.5 mg/kg to about 5 mg/kg, about 0.5 mg/kg to about 4.5 mg/kg, about 0.5 mg/kg to about 4 mg/kg, about 0.5 to about 3.5 mg/kg, about 0.5 mg/kg/day to about 3 mg/kg/day, about 0.5 mg/kg/day to about 2.5 mg/kg/day, about 0.5 mg/kg/day to about 2 mg/kg/day, about 0.5 mg/kg/day to about 1.5 mg/kg/day, about 0.5 mg/kg/day to about 1 mg/kg/day, about 1 mg/kg/day to about 5 mg/kg/day, about 1.5 mg/kg/day to about 5 mg/kg/day, about 2 mg/kg/day to about 5 mg/kg/day, about 2.5 mg/kg/day to about 5 mg/kg/day, about 3 mg/kg to about 5 mg/kg, about 3.5 mg/kg to about 5 mg/kg, about 4 mg/kg to about 5 mg/kg, about 4.5 mg/kg to about 5 mg/kg, about 1 mg/kg to about 4.5 mg/kg, about 1.5 mg/kg to about 4 mg/kg, about 2 mg/kg to about 3.5 mg/kg, or about 2.5 mg/kg to about 3 mg/kg.

In various aspects, the psychological disorder is a severe anxiety disorder. Examples of severe anxiety disorders include, but are not limited to generalized anxiety disorder (GAD), panic disorder, depression, depression with severe anxiety, and bipolar disorder with severe anxiety.

In various aspects, the psychological disorder is obsessive-compulsive disorder (OCD).

In various aspects, the psychological disorder is depression. In a further aspect, depression is treatment-resistant depression.

In various aspects, the subject has not sufficiently responded to at least one adequate antidepressant treatment prior to the administering step. Examples of adequate antidepressant treatments include, but are not limited to, administration of bupropion, a monoamine oxidase inhibitor, a SNRI, a SSRI, a TCA, nefazodone, trazodone, pramipexole, mirtazapine, or vortioxetine. In a further aspect, the adequate treatment comprises administration of an agent selected from the group consisting of a SSRI, a SNRI, a TCA, a MAOI, and a 5HT_{1A} receptor agonist.

In various aspects, the subject has not responded to at least two adequate antidepressant treatments prior to the administering step. In a further aspect, the two adequate antidepressant treatments comprise treatments with agents from two different classes of antidepressants. In a still further aspect, the subject has not responded to at least three adequate antidepressant treatments prior to the administering step. In a further aspect, the three adequate antidepressant treatments comprise treatments with agents from three different classes of antidepressants.

In various aspects, administering is via oral administration. In a further aspect, administering is via intranasal administration (e.g., via a nasal spray). In a still further aspect, administering is via transdermal administration (e.g., via a patch). In yet a further aspect, administering is via intradermal administration (e.g., via a microneedle array).

### F. Additional Methods of Using the Compositions

Provided are methods of using of a disclosed composition or medicament. In one aspect, the method of use is directed to the treatment of a disorder. In a further aspect, the disclosed compounds can be used as single agents or in combination with one or more other drugs in the treatment, prevention, control, amelioration, or reduction of risk of the aforementioned diseases, disorders and conditions for which the compound or the other drugs have utility, where the combination of drugs together are safer or more effective than either drug alone. The other drug(s) can be administered by a route and in an amount commonly used therefore, contemporaneously or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound can be more efficacious than either as a single agent.

The pharmaceutical compositions and methods of the present invention can further comprise other therapeutically active compounds as noted herein which are usually applied in the treatment of the above-mentioned pathological conditions.

### 1. Manufacture of a Medicament

In one aspect, the invention relates to a method for the manufacture of a medicament for treating a psychological disorder in a mammal, the method comprising combining a therapeutically effective amount of a compound having a structure represented by a formula:

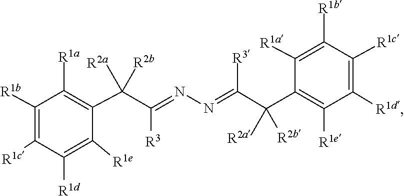

wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a}, R^{2a′}, R^{2b}, and R^{2b′} is independently selected from hydrogen, halogen, —OH, —NH₂, C1-C4 alkyl, and C2-C4 alkenyl; and wherein each of R³ and R^{3′} is independently selected from hydrogen and C1-C4 alkyl, or a pharmaceutically acceptable salt thereof, with a pharmaceutically acceptable carrier or diluent.

In one aspect, the invention relates to a method for the manufacture of a medicament for treating a psychological disorder in a mammal, the method comprising combining a therapeutically effective amount of a compound having a structure:

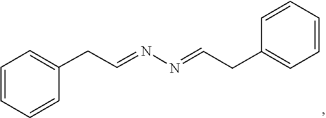 [SMILES: C(/Cc1ccccc1)=N\N=C\Cc1ccccc1]

or a pharmaceutically acceptable salt thereof, with a pharmaceutically acceptable carrier or diluent.

As regards these applications, the present method includes the administration to an animal, particularly a mammal, and more particularly a human, of a therapeutically effective amount of the compound effective in the treatment of a psychological disorder, such as severe anxiety, depression with severe anxiety, and other psychological disorders disclosed herein. The dose administered to an animal, particularly a human, in the context of the present invention should be sufficient to affect a therapeutic response in the animal over a reasonable time frame. One skilled in the art will recognize that dosage will depend upon a variety of factors including the condition of the animal, the body weight of the animal, as well as the severity and stage of the disorder.

Thus, in one aspect, the invention relates to the manufacture of a medicament comprising combining a compound having a structure represented by a formula:

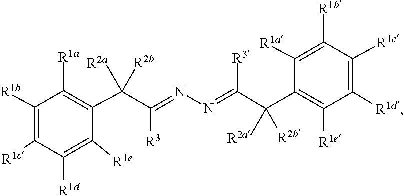

wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a}, R^{2a′}, R^{2b}, and R^{2b′} is independently selected from hydrogen, halogen, —OH, —NH₂, C1-C4 alkyl, and C2-C4 alkenyl; and wherein each of R³ and R^{3′} is independently selected from hydrogen and C1-C4 alkyl, or a pharmaceutically acceptable salt thereof, solvate, or polymorph thereof, optionally with an antidepressant, and a pharmaceutically acceptable carrier or diluent.

In a further aspect, the invention relates to the manufacture of a medicament comprising combining a compound having a structure:

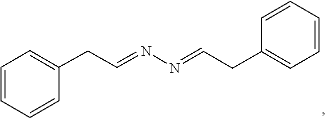 [SMILES: C(/Cc1ccccc1)=N\N=C\Cc1ccccc1]

or a pharmaceutically acceptable salt thereof, solvate, or polymorph thereof, optionally with an antidepressant, and a pharmaceutically acceptable carrier or diluent.

### 2. Use of Compounds and Compositions

Also provided are the uses of the disclosed compounds and compositions. Thus, in one aspect, the invention relates to the uses of a compound having a structure represented by a formula:

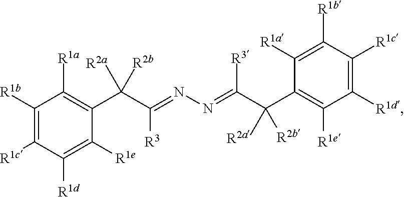

wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a}, R^{2a′}, R^{2b}, and R^{2b′} is independently selected from hydrogen, halogen, —OH, —NH₂, C1-C4 alkyl, and C2-C4 alkenyl; and wherein each of R³ and R^{3′} is independently selected from hydrogen and C1-C4 alkyl, or a pharmaceutically acceptable salt thereof, for treating psychological disorders. In a further aspect, the compound is:

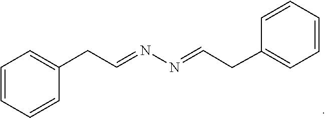 [SMILES: C(/Cc1ccccc1)=N\N=C\Cc1ccccc1]

In a further aspect, the invention relates to the use of a disclosed compound, or a pharmaceutically acceptable salt thereof, optionally in combination with an antidepressant, in the manufacture of a medicament for the treatment of a psychological disorder such as, for example, a severe anxiety disorder (e.g., generalized anxiety disorder (GAD), panic disorder, depression, treatment-resistant depression, depression with severe anxiety, and bipolar disorder with severe anxiety).

In a further aspect, the use relates to a process for preparing a pharmaceutical composition comprising a disclosed compound, or a pharmaceutically acceptable salt thereof, and a pharmaceutically acceptable carrier, for use as a medicament.

In a further aspect, the use relates to a process for preparing a pharmaceutical composition comprising a therapeutically effective amount of a compound having a structure:

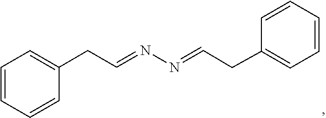 [SMILES: C(/Cc1ccccc1)=N\N=C\Cc1ccccc1]

or a pharmaceutically acceptable salt thereof, wherein a pharmaceutically acceptable carrier is intimately mixed with a therapeutically effective amount of the compound.

In various aspects, the use relates to the treatment of a psychological disorder in a vertebrate animal. In a further aspect, the use relates to the treatment of a psychological disorder in a human subject.

In a further aspect, the use is the treatment of a psychological disorder, for example, a severe anxiety disorder (e.g., generalized anxiety disorder (GAD), panic disorder, depression, treatment-resistant depression, depression with severe anxiety, and bipolar disorder with severe anxiety) or obsessive-compulsive disorder (OCD).

It is understood that the disclosed uses can be employed in connection with the disclosed compounds, methods, compositions, and kits. In a further aspect, the invention relates to the use of a disclosed compound or composition of a medicament for the treatment of a psychological disorder in a mammal.

In a further aspect, the invention relates to the use of a disclosed compound or composition in the manufacture of a medicament for the treatment of a psychological disorder such as, for example, a severe anxiety disorder (e.g., generalized anxiety disorder (GAD), panic disorder, depression, treatment-resistant depression, depression with severe anxiety, and bipolar disorder with severe anxiety) or obsessive-compulsive disorder (OCD).

### 3. Devices

In one aspect, disclosed are devices comprising: (a) a compound having a structure represented by a formula:

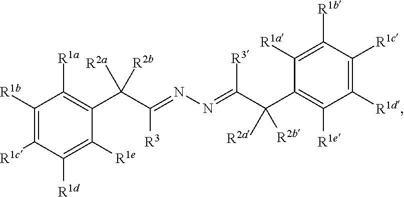

wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a}, R^{2a′}, R^{2b}, and R^{2b′} is independently selected from hydrogen, halogen, —OH, —NH₂, C1-C4 alkyl, and C2-C4 alkenyl; and wherein each of R³ and R^{3′} is independently selected from hydrogen and C1-C4 alkyl, or a pharmaceutically acceptable salt thereof; (b) a microneedle array or a transdermal patch; and (c) optionally, a transdermal agent.

In one aspect, disclosed are devices comprising: (a) a compound having a structure:

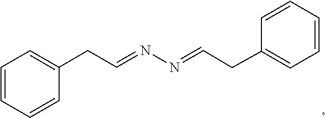 [SMILES: C(/Cc1ccccc1)=N\N=C\Cc1ccccc1]

or a pharmaceutically acceptable salt thereof; (b) a microneedle array or a transdermal patch; and (c) optionally, a transdermal agent.

In various aspects, the device comprises the microneedle array.

In various aspects, the device comprises the transdermal patch.

In various aspects, the devices comprises the transdermal agent. Examples of transdermal agents include, but are not limited to, is an alkyl myristate, a glycol, a surfactant, a terpene, an azone, a sulfoxide, and a pyrrolidone.

### 4. Kits

In one aspect, disclosed are kits comprising a compound having a structure represented by a formula:

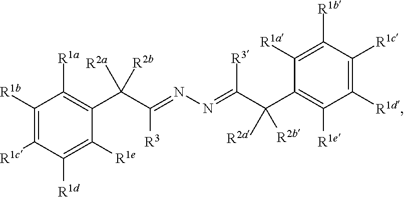

wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl; wherein each of R^{2a}, R^{2a′}, R^{2b}, and R^{2b′} is independently selected from hydrogen, halogen, —OH, —NH₂, C1-C4 alkyl, and C2-C4 alkenyl; and wherein each of R³ and R^{3′} is independently selected from hydrogen and C1-C4 alkyl, or a pharmaceutically acceptable salt thereof, and one or more selected from: (a) an antidepressant; (b) a device for delivering a medicament orally or intranasally; (c) a microneedle array or a transdermal patch; and (d) instructions for treating a psychological disorder.

In one aspect, disclosed are kits comprising a compound having a structure:

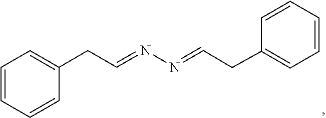 [SMILES: C(/Cc1ccccc1)=N\N=C\Cc1ccccc1]

or a pharmaceutically acceptable salt thereof, and one or more selected from: (a) an antidepressant; (b) a device for delivering a medicament orally or intranasally; (c) a microneedle array or a transdermal patch; and (d) instructions for treating a psychological disorder.

In various aspects, the kit comprises an antidepressant. Examples of antidepressants include, but are not limited to, selective serotonin reuptake inhibitors (SSRIs) (e.g., citalopram, dapoxetine, escitalopram, fluoxetine, fluvoxamine, paroxetine, sertraline, vortioxetine), serotonin-norepinephrine reuptake inhibitors (SNRIs) (e.g., desvenlafaxine, duloxetine, levomilnacipran, venlafaxine), tricyclic antidepressants (TCAs) (e.g., amitriptyline, amoxapine, desipramine, doxepin, imipramine, nortriptyline, protriptyline, and trimipramine), and 5HT_{1A} receptor agonists (e.g., buspirone, trazodone, nefazodone, vortioxetine, flibanserin, etappirone, lesopitron, alnespirone, repinotan, gepirone). In a further aspect, the kit comprises a 5HT_{1A} receptor agonist. In a still further aspect, the 5HT_{1A} receptor agonist is buspirone.

In various aspects, the kit comprises a device for delivering a medicament orally or intranasally. Examples of devices for delivering medicaments orally or intranasally include, but are not limited to, a teaspoon, a tablespoon, a medicine cup, a calibrated medicine spoon, a calibrated oral medicine dropper, an oral dosing syringe, an injectable syringe, an atomizer, and a medicine bottle.

In various aspects, the kit comprises a microneedle array or a transdermal patch.

In various aspects, the psychological disorder is a severe anxiety disorder. Examples of severe anxiety disorders include, but are not limited to generalized anxiety disorder (GAD), panic disorder, depression, depression with severe anxiety, and bipolar disorder with severe anxiety.

In various aspects, the psychological disorder is obsessive-compulsive disorder (OCD).

In various aspects, the psychological disorder is depression. In a further aspect, depression is treatment-resistant depression.

In various aspects, the compound and the antidepressant are co-packaged. In a further aspect, the compound and the antidepressant are co-formulated.

In some aspects, the kit further comprises a plurality of dosage forms, the plurality comprising one or more doses; wherein each dose comprises an effective amount of the compound and, optionally, the antidepressant agent. In a still further aspect, the effective amount is a therapeutically effective amount. In yet a further aspect, the effective amount is a prophylactically effective amount.

In some aspects, each dose of the compound and the antidepressant are co-formulated. In a still further aspect, each dose of the compound and the antidepressant are co-packaged.

In some aspects, the dosage forms are formulated for oral administration, inhalation, topical administration, and/or parenteral administration. In a still further aspect, the dosage form for the compound is formulated for oral administration and the dosage form for the antidepressant is formulated for parental administration. In yet a further aspect, the dosage form for the compound is formulated for parental administration and the dosage form for the antidepressant is formulated for oral administration. In an even further aspect, the dosage form for the compound is formulated for topical administration and the dosage form for the antidepressant is formulated for parental administration. In a still further aspect, the dosage form for the compound is formulated for parental administration and the dosage form for the antidepressant is formulated for topical administration. In yet a further aspect, the dosage form for the compound is formulated for oral administration and the dosage form for the antidepressant is formulated for inhalation. In an even further aspect, the dosage form for the compound is formulated for inhalation and the dosage form for the antidepressant is formulated for oral administration. In a still further aspect, the dosage form for the compound is formulated for topical administration and the dosage form for the antidepressant is formulated for inhalation. In a yet further aspect, the dosage form for the compound is formulated for inhalation and the dosage form for the antidepressant is formulated for topical administration.

It is understood that the disclosed kits can be prepared from the disclosed compounds, products, and pharmaceutical compositions. It is also understood that the disclosed kits can be employed in connection with the disclosed methods of using.

### 5. Subjects

In various aspects, the subject of the herein disclosed methods is a vertebrate, e.g., a mammal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

In some aspects of the disclosed methods, the subject has been diagnosed with a need for treatment prior to the administering step. In some aspects of the disclosed method, the subject has been diagnosed with a psychological disorder prior to the administering step. In some aspects of the disclosed methods, the subject has been identified with a need for treatment prior to the administering step. In one aspect, a subject can be treated prophylactically with a compound or composition disclosed herein, as discussed herein elsewhere.

### A. Dosage

Toxicity and therapeutic efficacy of the agents and pharmaceutical compositions described herein can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and can be expressed as the ratio LD₅₀/ED₅₀. Polypeptides or other compounds that exhibit large therapeutic indices are preferred.

Data obtained from cell culture assays and further animal studies can be used in formulating a range of dosage for use in humans. The dosage of such compounds lies preferably within a range of circulating concentrations that include the ED₅₀ with little or no toxicity, and with little or no adverse effect on a human's ability to hear. The dosage may vary within this range depending upon the dosage form employed and the route of administration utilized. For any agents used in the methods described herein, the therapeutically effective dose can be estimated initially from cell culture assays. A dose can be formulated in animal models to achieve a circulating plasma concentration range that includes the IC₅₀ (that is, the concentration of the test compound which achieves a half-maximal inhibition of symptoms) as determined in cell culture. Such information can be used to more accurately determine useful doses in humans. Exemplary dosage amounts of a differentiation agent are at least from about 0.01 to 3000 mg per day, e.g., at least about 0.00001, 0.0001, 0.001, 0.01, 0.1, 1, 2, 5, 10, 25, 50, 100, 200, 500, 1000, 2000, or 3000 mg per kg per day, or more.

The formulations and routes of administration can be tailored to the disease or disorder being treated, and for the specific human being treated. For example, a subject can receive a dose of the agent once or twice or more daily for one week, one month, six months, one year, or more. The treatment can continue indefinitely, such as throughout the lifetime of the human. Treatment can be administered at regular or irregular intervals (once every other day or twice per week), and the dosage and timing of the administration can be adjusted throughout the course of the treatment. The dosage can remain constant over the course of the treatment regimen, or it can be decreased or increased over the course of the treatment.

In various aspects, the dosage facilitates an intended purpose for both prophylaxis and treatment without undesirable side effects, such as toxicity, irritation or allergic response. Although individual needs may vary, the determination of optimal ranges for effective amounts of formulations is within the skill of the art. Human doses can readily be extrapolated from animal studies (Katocs et al., (1990) Chapter 27 in Remington's Pharmaceutical Sciences, 18th Ed., Gennaro, ed., Mack Publishing Co., Easton, PA). In general, the dosage required to provide an effective amount of a formulation, which can be adjusted by one skilled in the art, will vary depending on several factors, including the age, health, physical condition, weight, type and extent of the disease or disorder of the recipient, frequency of treatment, the nature of concurrent therapy, if required, and the nature and scope of the desired effect(s) (Nies et al., (1996) Chapter 3, In: Goodman & Gilman's The Pharmacological Basis of Therapeutics, 9th Ed., Hardman et al., eds., McGraw-Hill, New York, NY).

b. Routes of Administration

Also provided are routes of administering the disclosed compounds and compositions. The compounds and compositions of the present invention can be administered by direct therapy using systemic administration and/or local administration. In various aspects, the route of administration can be determined by a patient's health care provider or clinician, for example following an evaluation of the patient. In various aspects, an individual patient's therapy may be customized, e.g., the type of agent used, the routes of administration, and the frequency of administration can be personalized. Alternatively, therapy may be performed using a standard course of treatment, e.g., using pre-selected agents and pre-selected routes of administration and frequency of administration.

Systemic routes of administration can include, but are not limited to, parenteral routes of administration, e.g., intravenous injection, intramuscular injection, and intraperitoneal injection; enteral routes of administration e.g., administration by the oral route, lozenges, compressed tablets, pills, tablets, capsules, drops (e.g., ear drops), syrups, suspensions and emulsions; rectal administration, e.g., a rectal suppository or enema; a vaginal suppository; a urethral suppository; transdermal routes of administration; and inhalation (e.g., nasal sprays).

In various aspects, the modes of administration described above may be combined in any order.

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

### G. Examples

Without wishing to be bound by theory, it is believed that PEH may offer therapeutic benefits towards the treatment of various psychological disorders; however, its utility remains limited because it is unstable and purification is difficult. As detailed herein, the instant invention seeks to circumvent these shortcomings by administering PEH or a derivative thereof as a prodrug (i.e., an azine prodrug). As such, it is desirable not only to synthesize and purify the azine product, but also to confirm the conditions under which the azine converts to PEH. Further, by preparing PEH alongside the azine, it is possible to compare the stabilities and purification methodologies. The various synthetic protocols used to prepare the azine and PEH products are detailed below, as well as data characterizing, analyzing, and comparing these products.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

The Examples are provided herein to illustrate the invention, and should not be construed as limiting the invention in any way. Examples are provided herein to illustrate the invention and should not be construed as limiting the invention in any way.

### 1. Synthesis of PEH and Azine (Method 1)

PEH and the azine were synthesized according to the following general reaction scheme:

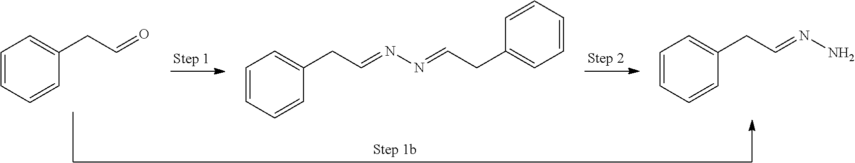 [SMILES: O=CCc1ccccc1 ; C(/Cc1ccccc1)=N\N=C\Cc1ccccc1 ; N/N=C/Cc1ccccc1]

### A. Step 1: Azine Formation

Reaction conditions for Batches 010 and 012-015 are shown in Table 1a and the results shown in Table 1b below.

TABLE 1A

| No. | Batch | Conditions |
|---|---|---|
| 1 | 1830-010 | Phenylacetaldehyde (0.5 g, 0.49 mL, 4.2 mmol), 1M |
| | | hydrazine in THF (0.067 g, 2.08 mL, 2.1 mmol), |
| | | DMSO-d₆ (2.5 mL), 100° C., 1 h |
| | | Work up: extraction DCM/H₂O |
| 2 | 1830-012 | Phenylacetaldehyde (1.5 g, 1.45 mL, 12.5 mmol), |
| | | ethanol (6.0 mL), hydrazine monohydrate (0.313 g, 0.3 |
| | | mL, 6.24 mmol), reflux (86-88° C.), 1 h |
| | | Work up: Addition of water (8 mL), extraction with |
| | | chloroform (3 × 20 mL) |
| 3 | 1830-014¹ | Phenylacetaldehyde (5.0 g, 4.8 mL, 41.6 mmol), |
| | | ethanol (20.0 mL), hydrazine monohydrate (1.04 g, |
| | | 1.0 mL, 20.8 mmol), reflux (80° C.), 1 h |
| | | Work up: Addition of water (20 mL), extraction with |
| | | chloroform |
| | | Purification: Trituration with cold ethanol was |
| | | performed |
| 4 | 1830-015 | Phenylacetaldehyde (1.5 g, 1.45 mL, 12.5 mmol), |
| | | ethanol (6.0 mL), hydrazine monohydrate (0.313 g, 0.3 |
| | | mL, 6.24 mmol), reflux (86-88° C.), 1 h |
| | | Work up: Addition of water (8 mL), extraction with |
| | | chloroform |
| 10 | 1830-013¹ | Phenylacetaldehyde (5.0 g, 4.8 mL, 41.6 mmol), |
| | | ethanol (20.0 mL), hydrazine monohydrate (1.04 g, |
| | | 1.0 mL, 20.8 mmol), reflux (89° C.), 1 h |
| | | Work up: Addition of water (20 mL), extraction with |
| | | chloroform |
| | | Purification: Trituration with cold ethanol was |
| | | performed |

¹In CDCl₃ NMR showed no characteristic azine signals and signal from water, but NMR in DMSO and MeOD confirmed the structure of the azine.

TABLE 1B

| No. | Amount | Purity³ | Yield² | Comments |
|---|---|---|---|---|
| 1 | Not | — | — | Reaction progress was |
| | isolated | | | measured by NMR and |
| | | | | UPLC. |
| 2 | 1.480 g | UPLC: 8% | 73-82% | NMR performed in |
| | | contamination | | deuterated CAN, py, |
| | | with PEH | | CDCl₃, MeOD, C₆D₆, |
| | | NMR: depends on | | THF-d₈. |
| | | deuterated solvent | | |
| | | used (73-82% of | | |
| | | azine) | | |
| 3 | 4.470 g | Crude product: | 91% | After trituration of |
| | | UPLC: 80% of | | 3.6 g, obtained mass |
| | | azine | | of pure azine: m = |
| | | NMR²: 100% of | | 1.963 g; NMR performed |
| | | azine | | in MeOD and DMSO-d₆. |
| | | After trituration: | | |
| | | UPLC: 100% of | | |
| | | azine | | |
| | | NMR: 100% of | | |
| | | azine | | |
| 4 | 1.590 g | Crude product: | 95% | NMR performed in |
| | | UPLC: 84% of | | CDCl₃. |
| | | azine | | |
| | | NMR: 88% of azine | | |
| 5 | 4.767 g | Crude product: | 97% | NMR performed in |
| | | NMR: 100% of | | DMSO-d₆. |
| | | azine (no PEH | | |
| | | signals) | | |

¹Yields were estimated acc. to ¹H NMR spectrum.
²NMR performed 2.5 weeks after product was obtained.
³UPLC was performed in basic conditions (in acidic conditions - decomposition of the azine was observed).

### (i) Synthesis of 1830-013

The azine was synthesized according to the procedure in Pross and Sternhell, Aust. J. Chem., 1970, 989-1003. Hydrazine monohydrate (1.04 g, 1.0 mL, 20.8 mmol) was added to a solution of phenylacetaldehyde (5.0 g, 4.8 mL, 41.6 mmol) in ethanol (20.0 mL). The mixture was then refluxed for 1 hour. At about 60° C. the color of the solution turned yellow. Water (20 mL) was added to the cooled mixture (cooling time: 20 min), which was then extracted with chloroform. The chloroform layer was washed with water, dried over anhydrous potassium carbonate, and filtered (extraction-filtration time: 30 min). The filtrate was evaporated (20 min, 35° C.) and the obtained yellow oil was dried (15 min) under vacuum to obtain 4.77 g of crude product (yield: 97%, LCMS 88.02%). The azine (4.0 g) was purified via trituration with small volume (15 mL) of cold ethanol to obtain a yellowish solid, 2.5 g, yield: 62%.

### (II) Analysis of Reaction Conditions

Batch 1830-010. Neither azine or PEH was detected for sample 1830-10 via chromatography or NMR analysis (data not shown).

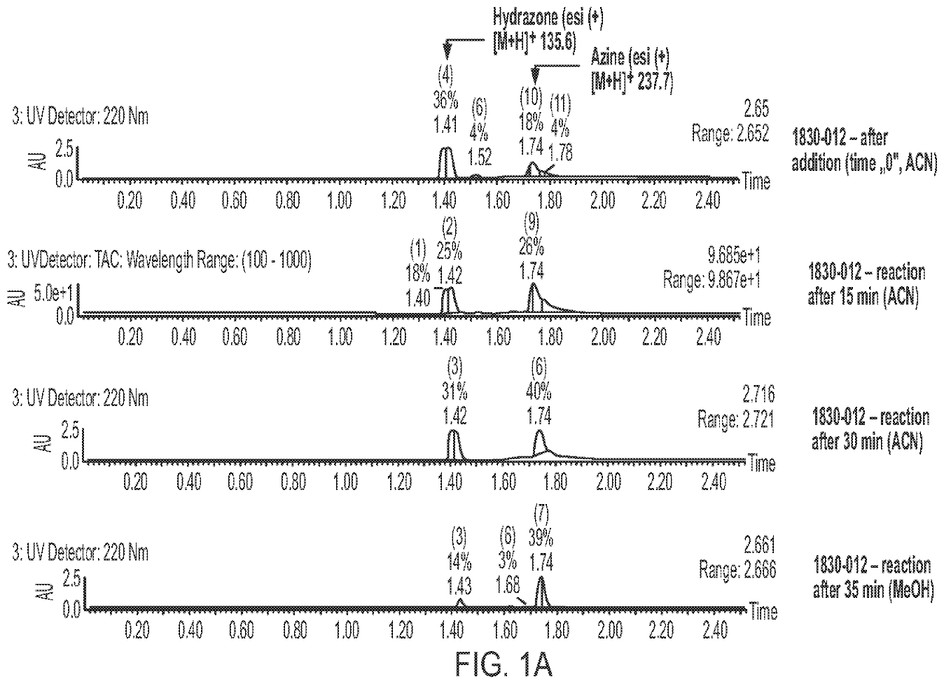

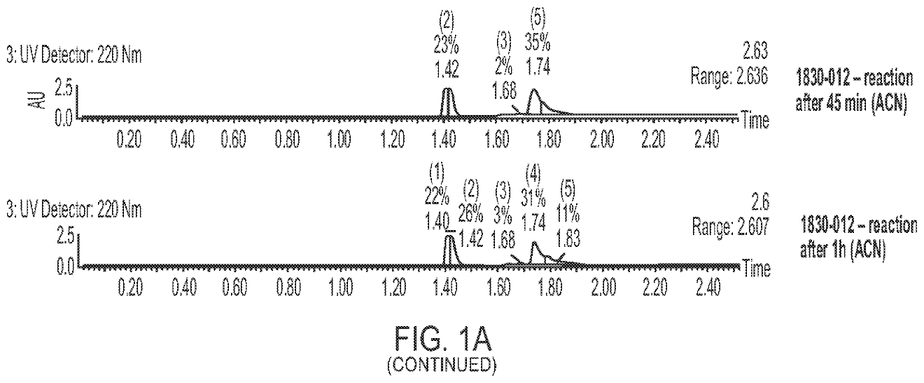

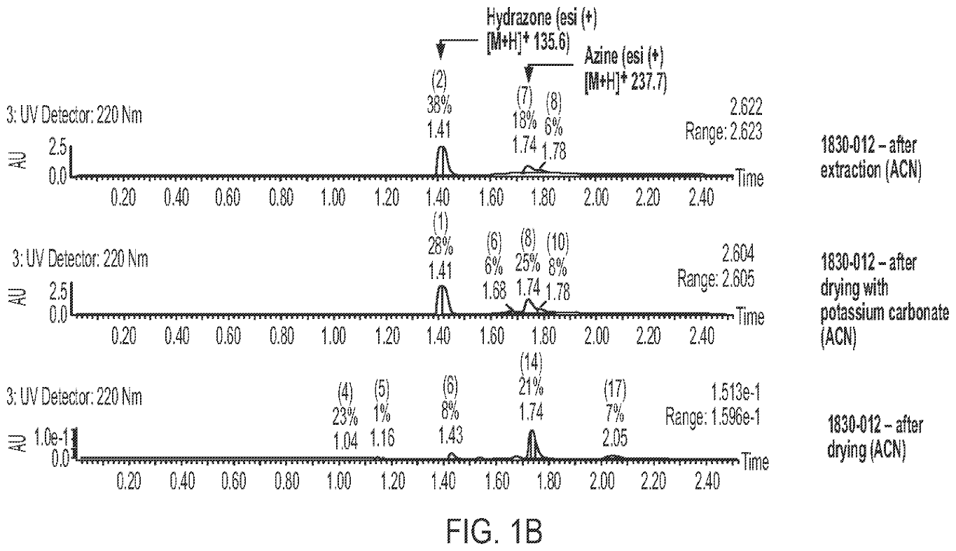

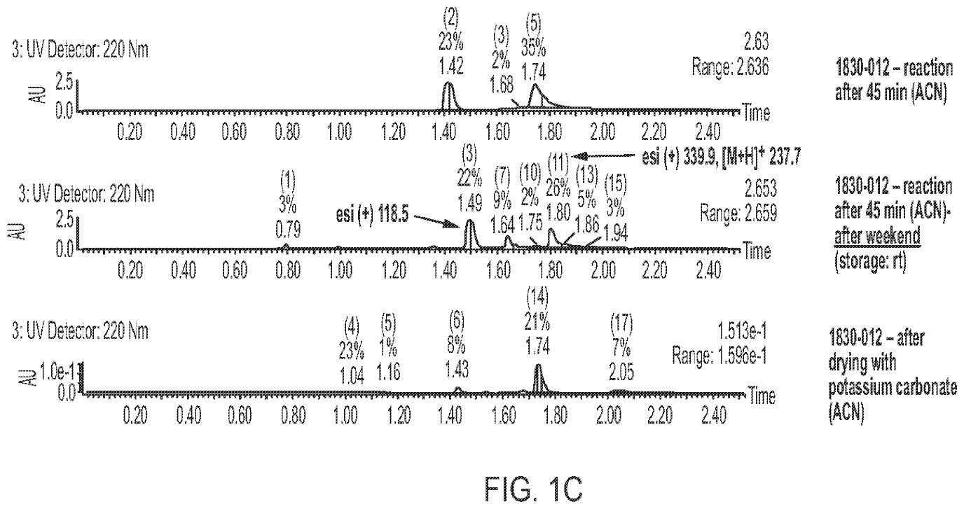

Batch 1830-012. UPLC chromatograms for sample 1830-012 were measured in ACN or MeOH directly after preparation. The UPLC method parameters were the same as those for PEH. FIG. 1A-C are chromatograms of sample 1830-012 obtained at different points in the reaction. FIG. 1A, top panel, is the chromatogram obtained at time “0,” and confirms the presence of PEH and the azine at [M+H]⁺ 135.6 and 237.7, respectively. As the reaction proceeds, the growth of the peak corresponding to the azine increases until 35 min. FIG. 1B is the chromatogram obtained in ACN after extraction (top panel) and after drying with potassium carbonate (middle panel). In both chromatograms, the signal due to the azine decreased and peak broadening was observed. FIG. 1B, bottom panel, is the chromatogram after drying, in which the signal due to PEH decreased. The chromatograms in FIG. 1C of the resultant species were obtained after 3 days storage at room temperature and −20° C. The presence of various impurities, including unidentified compounds at m/z 339.9 and 118.5, was observed. Without wishing to be bound by theory, these data suggest that PEH is likely unstable after 3 days.

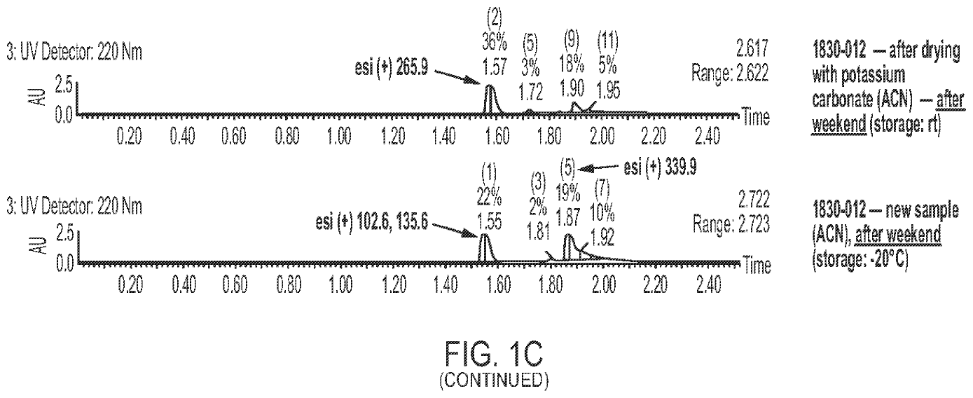

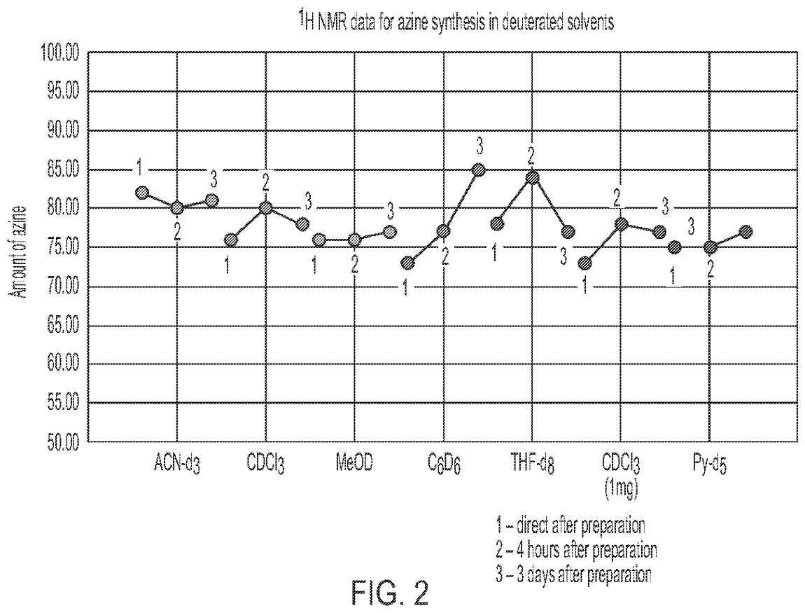

¹H NMR was employed to probe the stability of azine sample 1830-012 both in terms of time after preparation and in different deuterated solvents. The results are illustrated in FIG. 2 and summarized in Table 2 below.

TABLE 2

| | | PEH:Azine | | |
|---|---|---|---|---|
| Deuterated | Mass of | Direct after | 4 h after | 3 days after |
| solvent | Sample (mg) | preparation | preparation | preparation |
| ACN-d₃ | 2.9 | 18:82* | 20:80* | 19:81 |
| CDCl₃ | 2.9 | 24:76 | 20:80 | 22:78 |
| MeOD | 3.1 | 24:76 | 24:76 | 23:77 |
| C₆D₆ | 2.9 | 27:73* | 23:77* | 15:85 |
| THF-d₈ | 3.0 | 22:78 | 16:84 | 23:77 |
| CDCl₃ | 1.0 | 27:73 | 22:78 | 23:77 |
| Py-d₅ | 2.9 | 25:75* | 25:75* | 23:77 |

*Estimated values (signals from PEH overlapped with unidentified signals)

The ratio of PEH:Azine was similar for all deuterated solvents except for C₆D₆ 3 days after preparation, in which case the amount of azine increased.

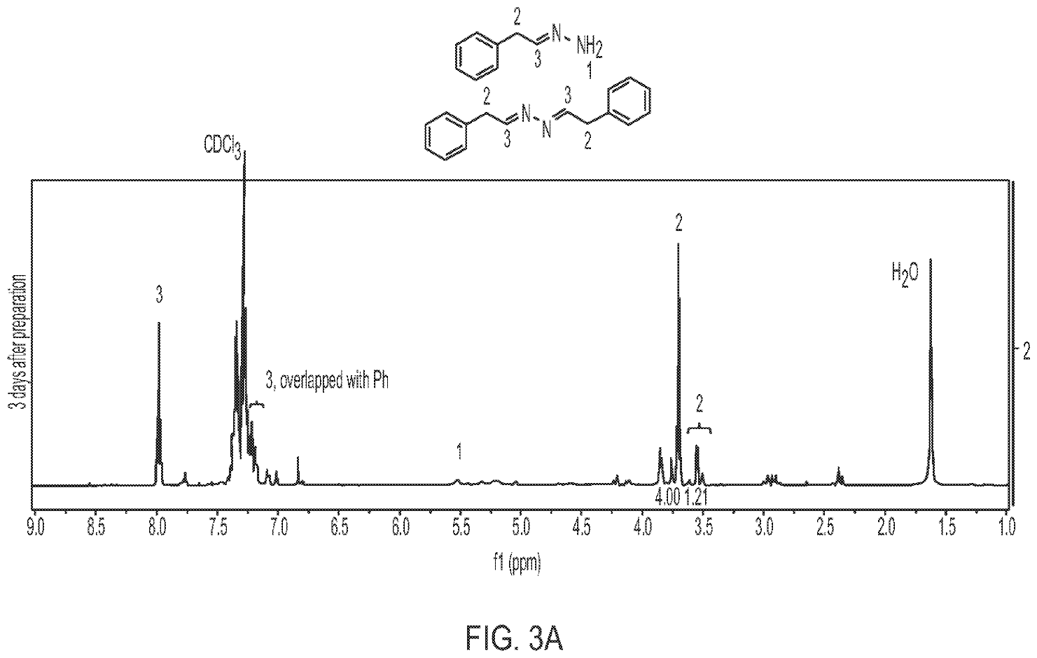

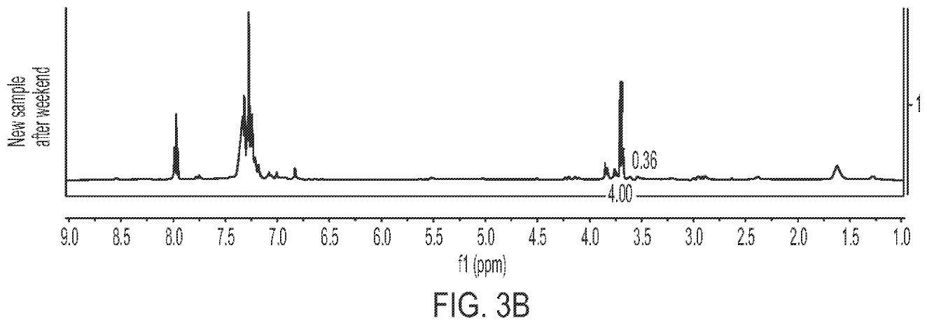

Additionally, a new sample from the oil obtained in sample 1830-012, in which the oil was prepared after 3 days storage in −20° C. under argon, was prepared and measured directly in CDCl₃. These spectra are shown in FIG. 3A and FIG. 3B.

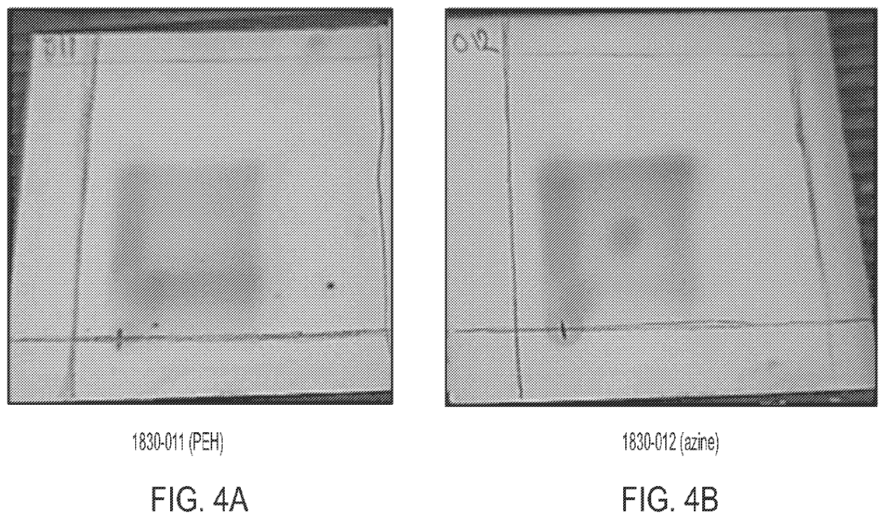

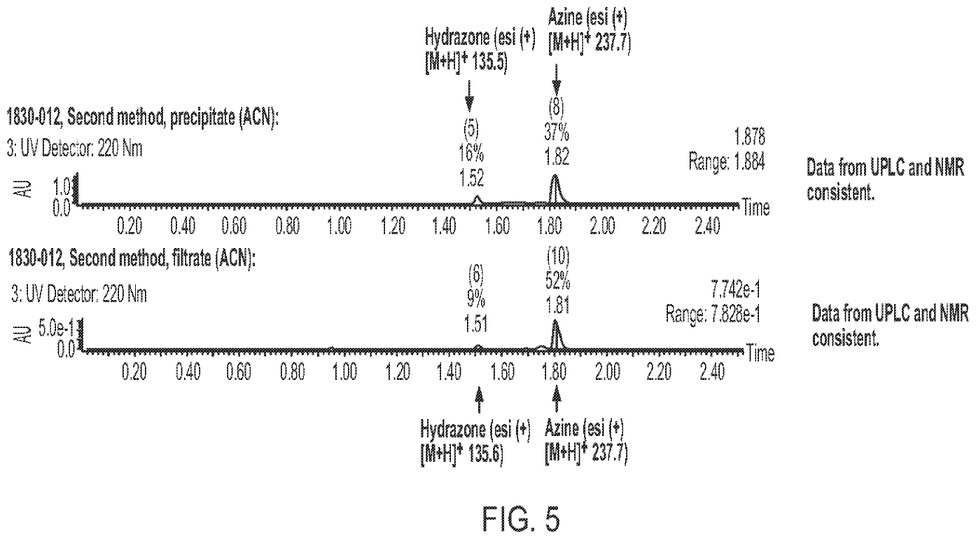

After 3 days, the contamination of the azine with PEH was equal to 9% (the ratio of PEH:Azine was 9:91). 2D TLC analysis (Hex:AcOEt=6:1, TLC Silica gel 60 NH₂F₂₅₄s) (FIG. 4A and FIG. 4B) shows that neither PEH (1830-011) nor the azine (1830-012) are stable in basic SiO₂. Thus, it is theorized that purification cannot be accomplished by column chromatography. Similarly, it was also found that crystallization did not allow for separation of the PEH and the azine—both the filtrate and the precipitate contained the azine with PEH contamination. First, EtOH (2.0 mL) was added to 300 mg of crude product 1830-012 and heated to reflux. During refluxing, ˜1.0 mL of EtOH evaporated. The mixture was then slowly cooled to room temperature and then cooled to 0° C. After 3 days at 0° C., no precipitate was observed. In a second attempt, cold EtOH (3.0 mL) was added to 600 mg of crude product 1830-012 and the suspension was stirred at 0° C. for 15 minutes. The white precipitate was filtered (m=131 mg) and washed with small amounts of cold EtOH (see FIG. 5).

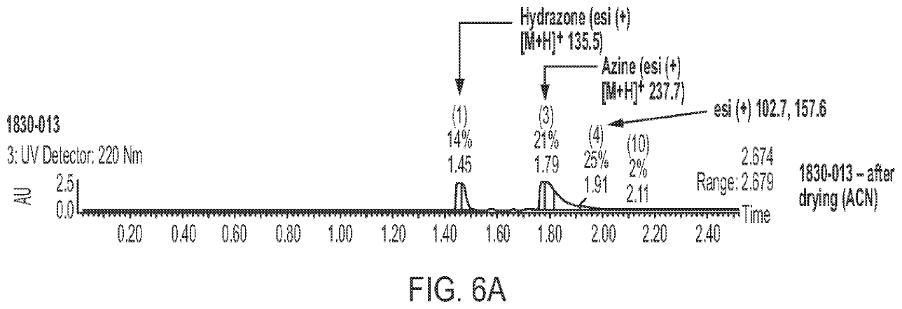

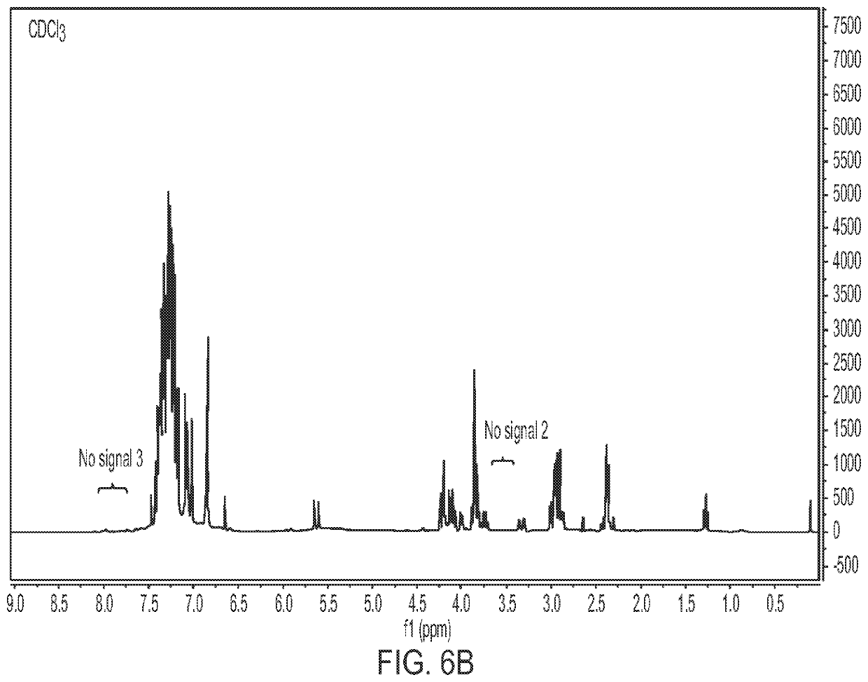

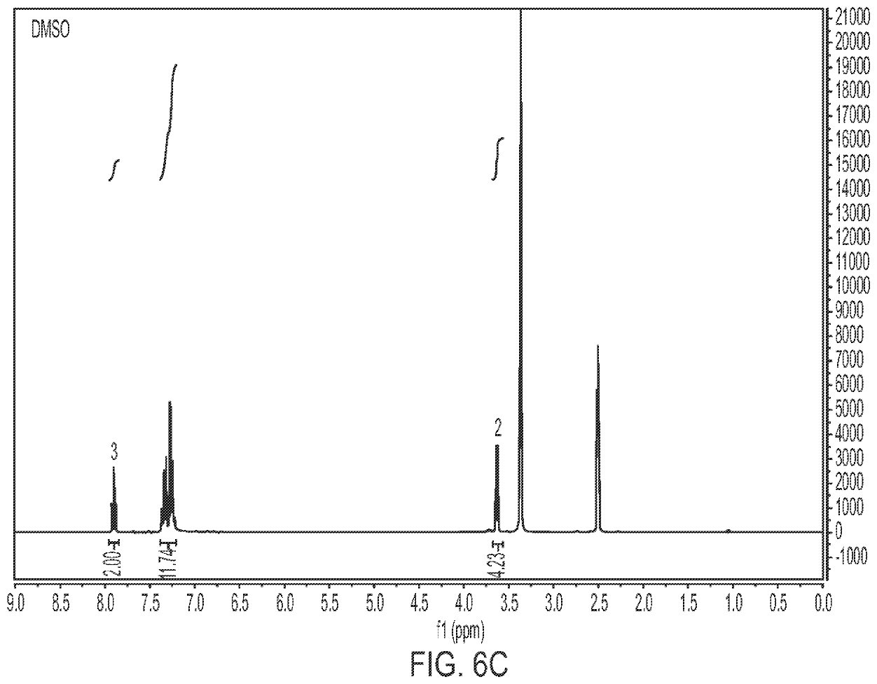

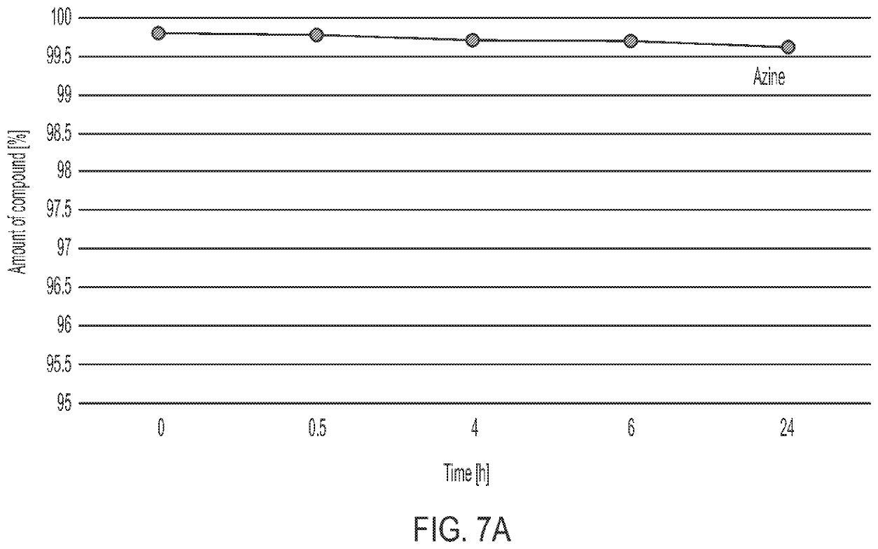

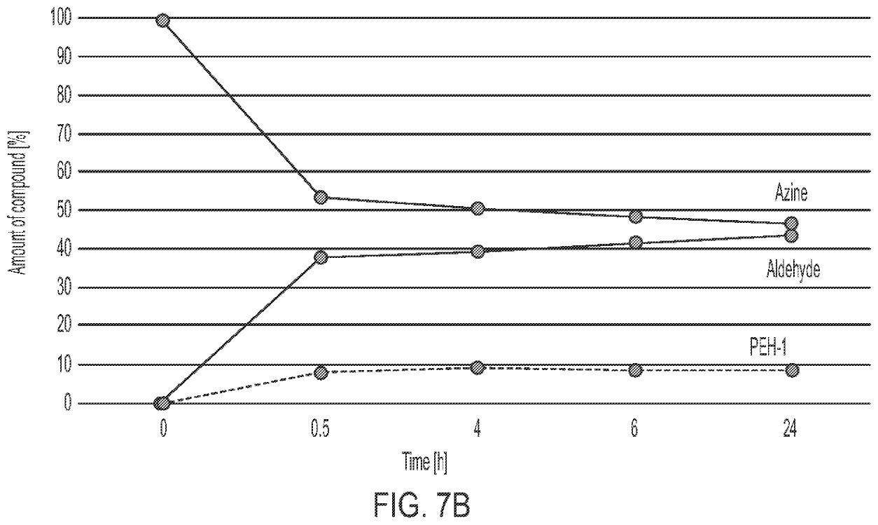

Batch 1830-013. The UPLC and ¹H NMR data for reaction 1830-013 are shown in FIG. 6A-C. Although UPLC confirmed the presence of PEH and the azine (FIG. 6A), no signals due to either species were detected in the NMR in CDCl₃ (FIG. 6B). NMR taken in DMSO-d₆ revealed signals for the azine only (FIG. 6C). pH stability studies indicated that the azine is stable at pH 9.2 (FIG. 7A). No signals for the aldehyde or PEH were observed at pH 9.2. FIG. 7B shows the distribution of compounds at pH 7.4. Similar to PEH, the azine undergoes rapid hydrolysis to the aldehyde at pH 7.4.

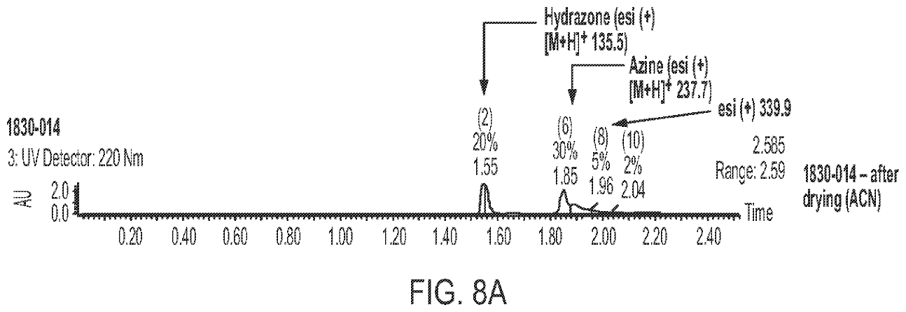

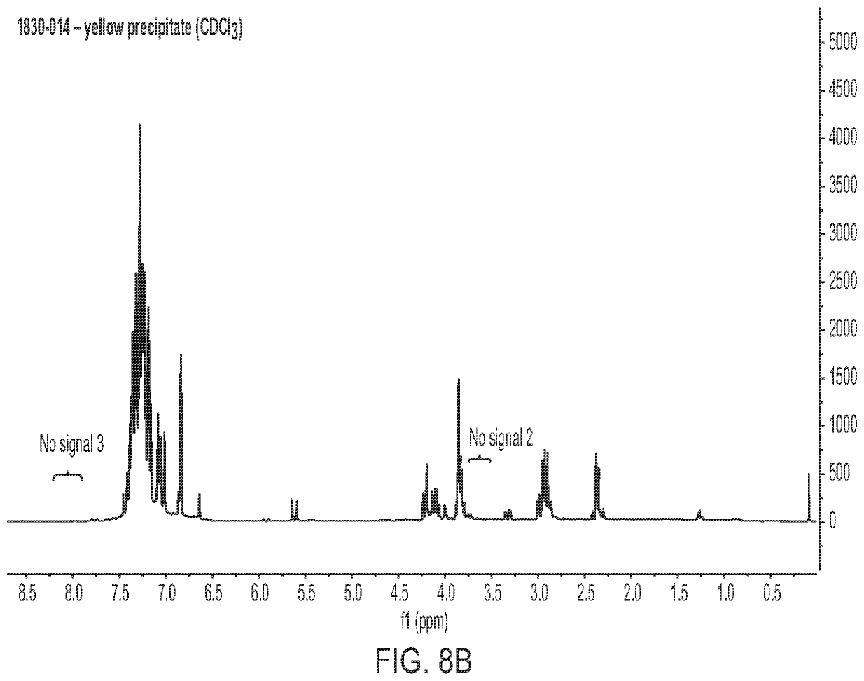

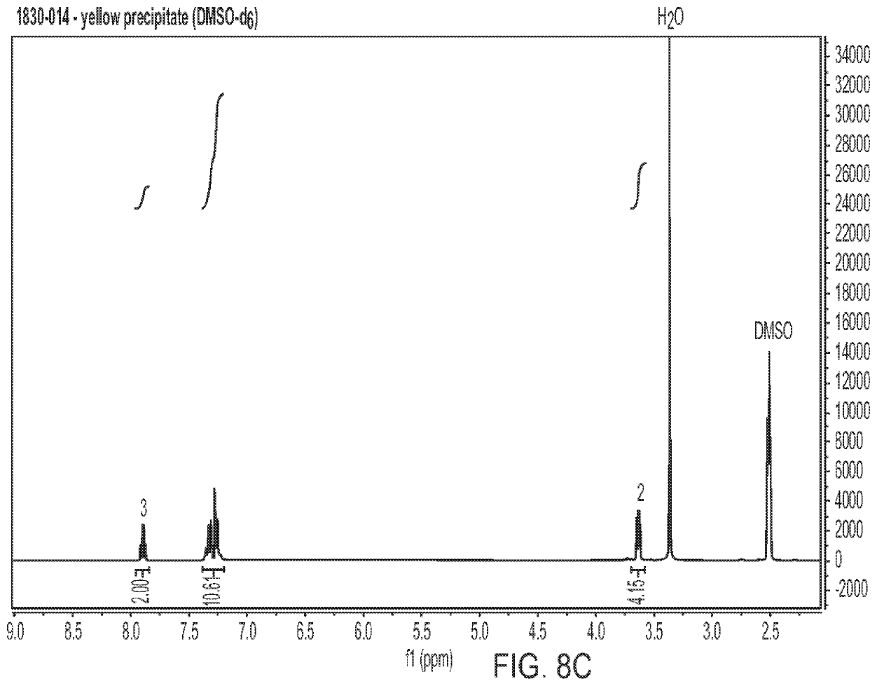

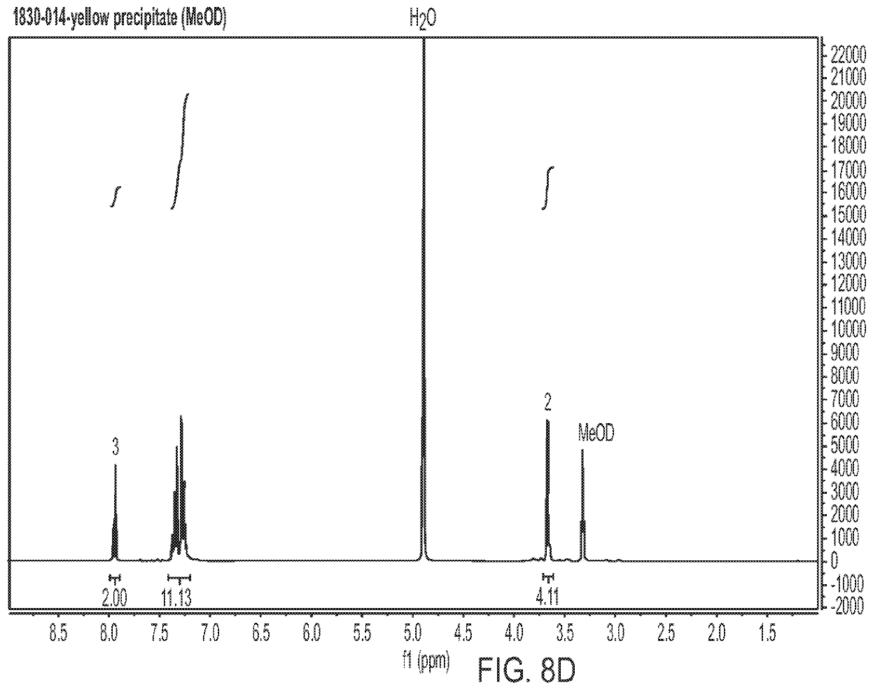

Batch 1830-014. The UPLC and ¹H NMR data for reaction 1830-014 are shown in FIG. 8A-D. Again, the presence of PEH and the azine was detected in the chromatography (FIG. 8A), but not the NMR in CDCl₃ (FIG. 8B). In DMSO-d₆ (FIG. 8C) and MeOD (FIG. 8D) NMR signals from the azine only were observed.

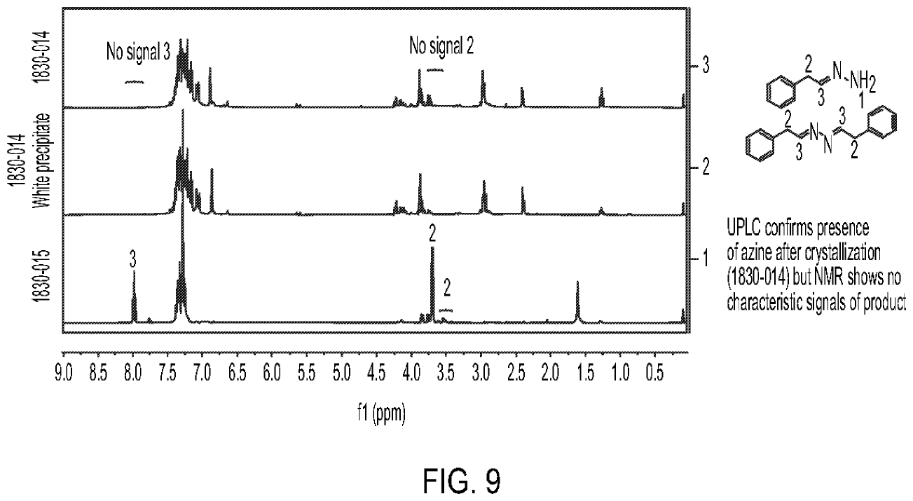

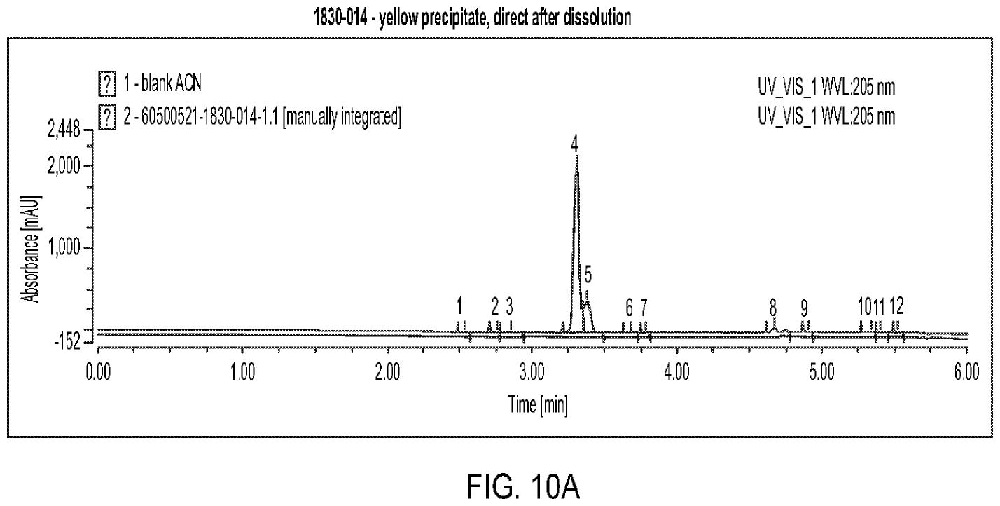

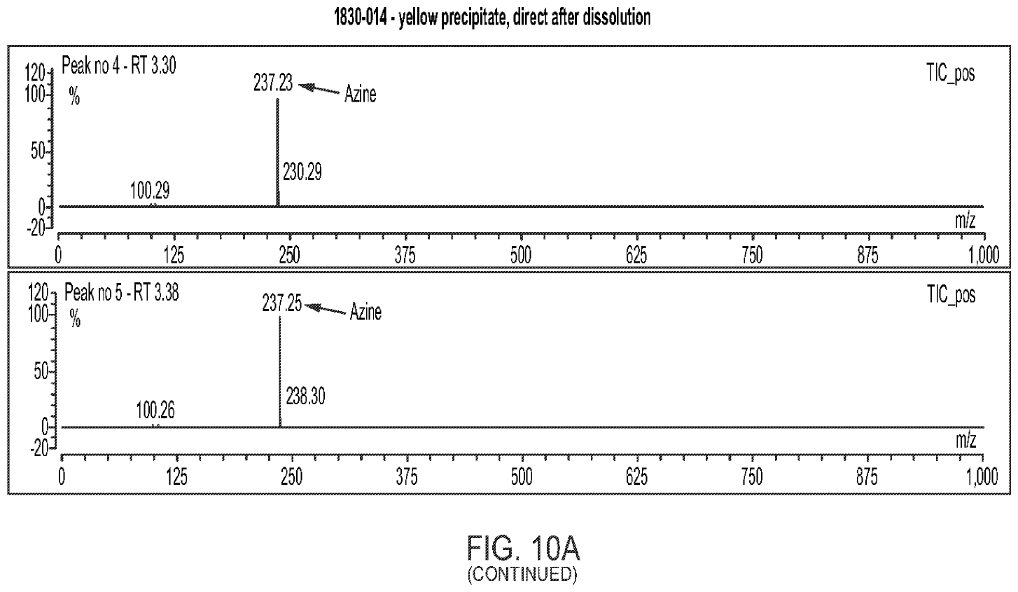

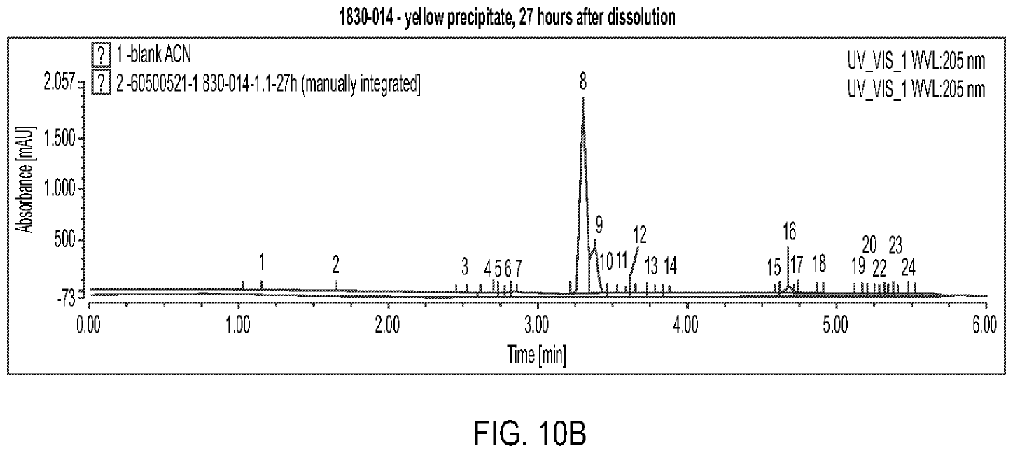

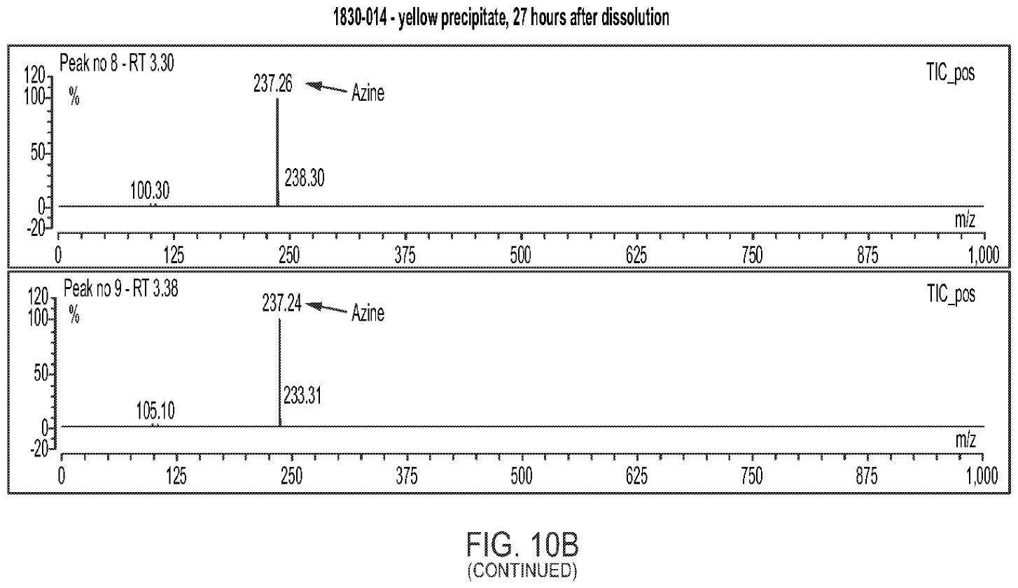

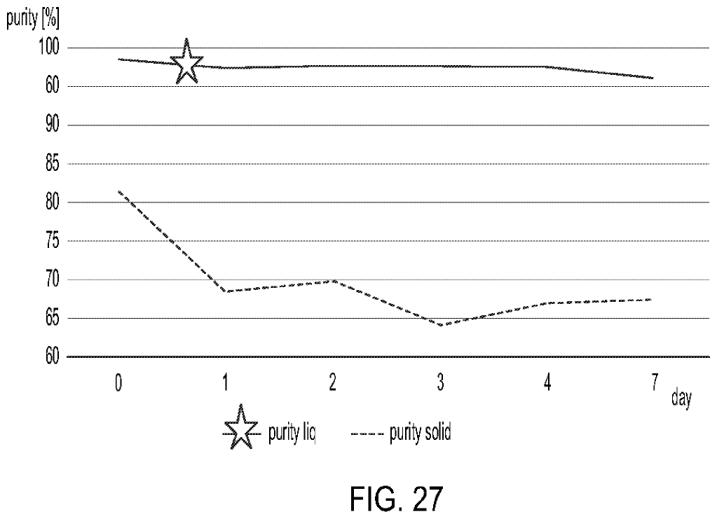

Crystallization was performed on 3.6 g of the crude azine product for 1830-014 using the second method as described above for 1830-012 (FIG. 9). White (m=0.490 g) and yellow (m=1.473 g) precipitates were obtained. LC-MS analysis was also performed on the azine sample obtained as a yellow precipitate (1830-014), with chromatograms obtained directly after dissolution (FIG. 10A) and 27 hours after dissolution (FIG. 10B). The quantitative results are described in Table 12 and Table 13 below. According to the LC-MS analysis using % relative area corresponding to the appropriate MS peaks, the azine sample obtained as a yellow precipitate was 96.24% azine. 27 hours after dissolution, the sample was 91.77% azine. After one month of storage at −20° C. under Argon, the sample was 91.56% azine (data not shown).

TABLE 3A

| Peak | Time | Area | Height | Relative |
|---|---|---|---|---|
| No. | (min) | (MAU*min) | (mAU) | Area % |
| 1 | 2.530 | 0.090 | 2.357 | 0.07 |
| 2 | 2.750 | 0.170 | 4.192 | 0.14 |
| 3 | 2.857 | 1.111 | 13.692 | 0.92 |
| 4 | 3.303 | 96.350 | 2037.573 | 79.95 |
| 5 | 3.380 | 19.629 | 382.023 | 16.29 |
| 6 | 3.677 | 0.133 | 2.227 | 0.11 |
| 7 | 3.783 | 0.088 | 2.367 | 0.07 |
| 8 | 4.673 | 2.184 | 38.396 | 1.81 |
| 9 | 4.903 | 0.103 | 3.115 | 0.09 |
| 10 | 5.343 | 0.371 | 5.980 | 0.31 |
| 11 | 5.403 | 0.205 | 4.000 | 0.17 |
| 12 | 5.530 | 0.073 | 1.792 | 0.06 |

TABLE 3B

| Peak | Time | Area | Height | Relative |
|---|---|---|---|---|
| No. | (min) | (MAU*min) | (mAU) | Area % |
| 1 | 1.147 | 0.751 | 7.939 | 0.60 |
| 2 | 1.647 | 0.045 | 0.000 | 0.04 |
| 3 | 2.533 | 0.279 | 5.957 | 0.22 |
| 4 | 2.707 | 1.016 | 21.435 | 0.82 |
| 5 | 2.750 | 0.597 | 15.239 | 0.48 |
| 6 | 2.827 | 0.532 | 17.886 | 0.43 |
| 7 | 2.860 | 1.447 | 27.500 | 1.16 |
| 8 | 3.303 | 90.687 | 1863.742 | 73.01 |
| 9 | 3.383 | 23.302 | 452.411 | 18.76 |
| 10 | 3.453 | 0.100 | 3.600 | 0.08 |
| 11 | 3.587 | 0.180 | 4.509 | 0.14 |
| 12 | 3.653 | 0.647 | 12.477 | 0.52 |
| 13 | 3.787 | 0.180 | 4.756 | 0.17 |
| 14 | 3.873 | 0.063 | 1.420 | 0.05 |
| 15 | 4.623 | 0.138 | 3.077 | 0.11 |
| 16 | 4.673 | 2.819 | 69.940 | 2.27 |
| 17 | 4.743 | 0.613 | 17.425 | 0.49 |
| 18 | 4.900 | 0.108 | 2.948 | 0.09 |
| 19 | 5.163 | 0.097 | 1.909 | 0.08 |
| 20 | 5.247 | 0.090 | 2.139 | 0.07 |
| 21 | 5.303 | 0.119 | 3.096 | 0.10 |
| 22 | 5.340 | 0.160 | 3.519 | 0.13 |
| 23 | 5.400 | 0.158 | 3.125 | 0.13 |
| 24 | 5.517 | 0.061 | 1.150 | 0.05 |

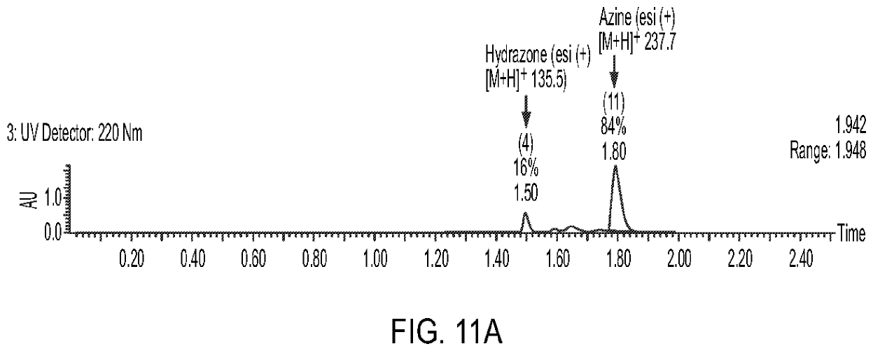

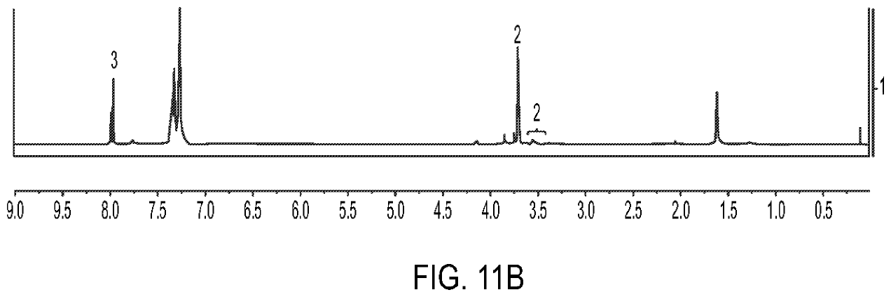

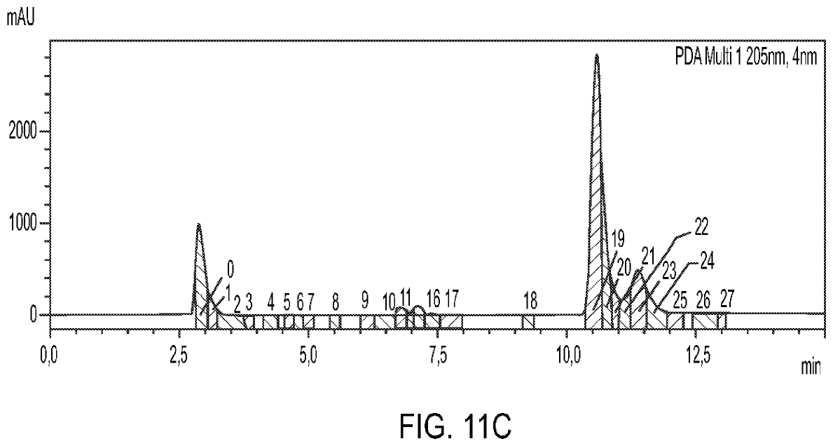

Batch 1830-015. Azine (1830-015) was analyzed using UPLC immediately before subjecting the sample to preparative HPLC. The chromatographic run is shown in FIG. 11A, and shows that the sample contained both PEH ([M+H]+ 135.5) and azine ([M+H]⁺ 237.7), as obtained by positive mode ESI. The ¹H NMR spectrum obtained in CDCl₃ is shown in FIG. 11B, and confirmed that both compounds are present in the azine sample according to the proton as-signments, although the signals of protons 1 and 3 of PEH were not detected. The ratio of Azine: PEH as determined by NMR was 88:12. Preparative HPLC was performed on 19.7 mg of solid sample in 0.4 mL acetonitrile on a reverse phase C18 column with a mobile phase of H₂O+0.05% NH₃ and acetonitrile. The method duration was 15 minutes, and the purification duration was 1 hour. The chromatogram obtained from the preparative HPLC purification is shown in FIG. 11C, and shows the two desired peaks at 10.6 min (first fraction) and 11.4 min (second fraction). The pump parameters were as described in Table 4 below.

TABLE 4

| | |
|---|---|
| Mode | Binary gradient |
| Pump A | LC-20 AP |
| Pump B | LC-20 AP |
| Total Flow | 30.00 mL/min |
| B. Conc. | 54.0% |
| B Curve | 0 |
| Maximum Pressure | 42.0 MPa |
| Minimum Pressure | 0 MPa |
| Solenoid Valve B Name | FCV-200AL |
| Solenoid Valve B | A |
| Compressibility Setting | On |
| Pump A Compressibility | 0.45/GPa |
| Pump B Compressibility | 1.20/GPa |

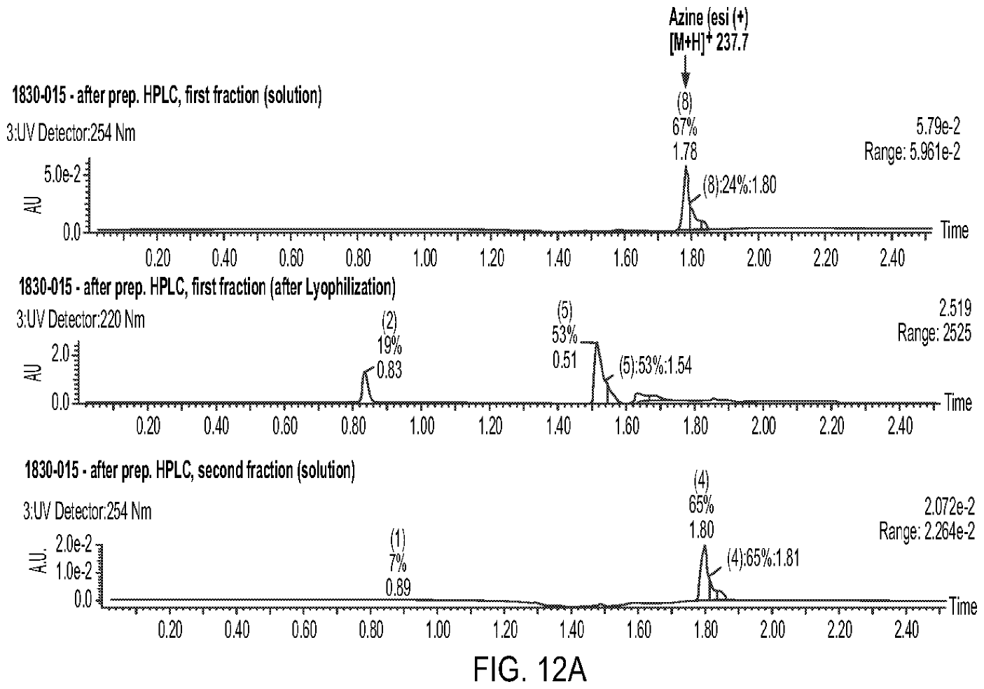

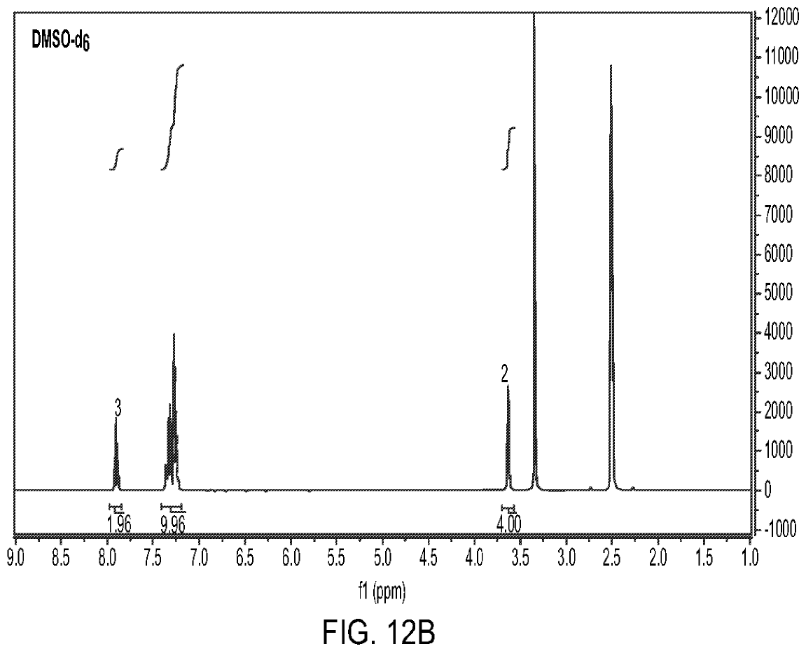

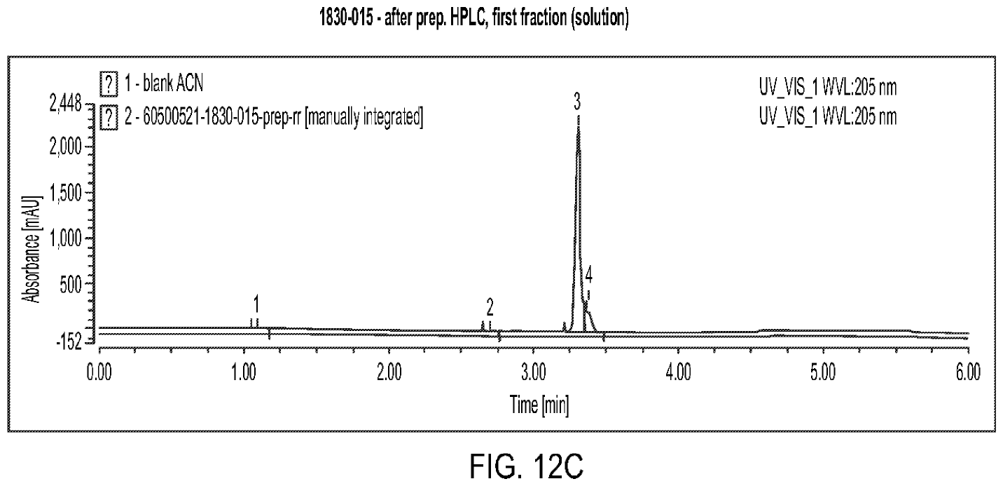

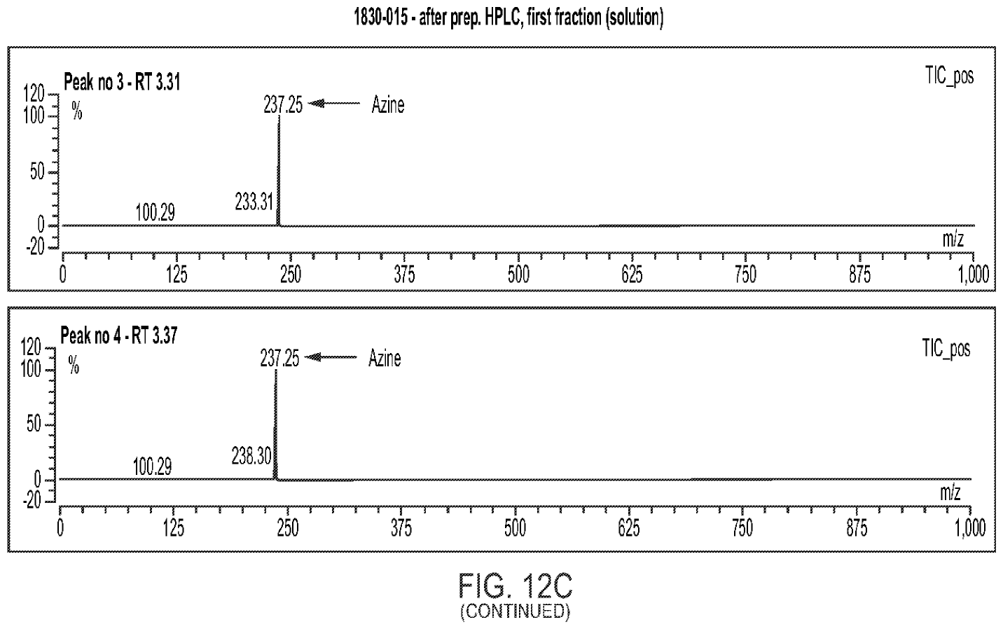

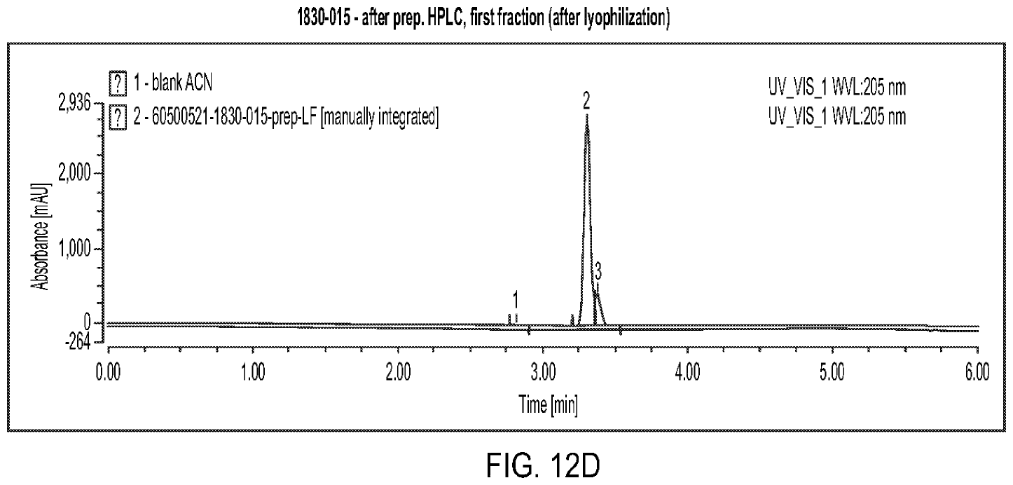

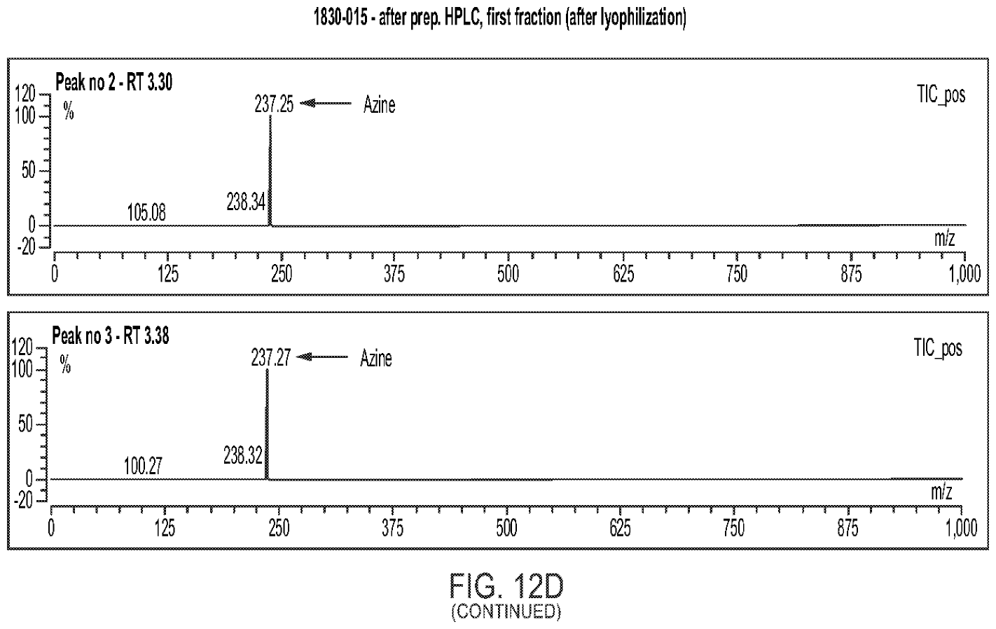

The chromatographic runs of the fractions obtained by purification are shown in FIG. 12A. The top panel is the chromatogram of the first fraction in solution phase, and the peak corresponding to the azine is observed at [M+H]⁺=237.7 m/z. The middle panel is the chromatogram of the first frac-tion after lyophilization. The bottom panel is the chromatogram of the second fraction in solution phase, and once again the peak (237.7 m/z) corresponding to the azine was observed. Without wishing to be bound by theory, this suggests that the two fractions obtained from the azine sample are likely azine isomers. The ¹H NMR spectrum in DMSO-d₆ of the first fraction after lyophilization is shown in FIG. 12B. Only signals corresponding to azine protons 2 and 3 were observed, suggesting a stable and relatively pure sample of azine after lyophilization. The results obtained for the LC-MS analysis of the first fraction of the azine sample in solution and after lyophilization are shown in FIG. 12C and FIG. 12D, respectively. The quantitative results are in Table 5a and Table 5b below. According to the LC-MS analysis using % relative area corresponding to the appropriate MS peaks, the first fraction of the azine sample after preparative HPLC was 99.61% azine (Table 5a, row nos. 3 and 4). Using a similar analysis for the first fraction of the azine sample after lyophilization, the lyophilized sample was 99.93% azine (Table 5b, row nos. 2 and 3), indicating that azine remained stable after lyophilization, and purification of >99% azine was obtained using preparative HPLC.

TABLE 5A

| Peak | Time | Area | Height | Relative |
|---|---|---|---|---|
| No. | (min) | (MAU*min) | (mAU) | Area % |
| 1 | 1.100 | 0.387 | 7.089 | 0.33 |
| 2 | 2.707 | 0.072 | 1.526 | 0.06 |
| 3 | 3.307 | 108.895 | 2247.671 | 91.86 |
| 4 | 3.370 | 9.185 | 234.699 | 7.75 |

TABLE 5B

| Peak | Time | Area | Height | Relative |
|---|---|---|---|---|
| No. | (min) | (MAU*min) | (mAU) | Area % |
| 1 | 2.817 | 0.100 | 1.487 | 0.07 |
| 2 | 3.303 | 130.079 | 2687.708 | 85.73 |
| 3 | 3.380 | 21.545 | 429.020 | 14.20 |

### (iii) Analytics

### UPLC Method.

b. Step 1B/2: PEH Formation

Reaction conditions for Batches 001 to 004, 006 to 007, 011, 016, and 018 are shown in Table 6a and the results shown in Table 6b below.

TABLE 6A

| No. | Step | Batch | Conditions |
|---|---|---|---|
| 1 | 1b | 1830-001 | 0.2 g SM, 0.8 mL (0.5 eq) 1M hydrazine in THF, |
| | | | absolute EtOH [8.2*10⁻⁴ M], refluxed for 1.5 hours - |
| | | | hydrazine was added to aldehyde |
| 2 | 1b | 1830-002 | 0.2 g SM, 0.8 mL (0.5 eq) 1M hydrazine in THF, |
| | | | absolute EtOH [8.2*10⁻⁴ M], room temperature for 1.5 |
| | | | hours - hydrazine was added to aldehyde |
| 3 | 1b | 1830-003A¹ | 0.2 g SM, 5.0 mL (3.0 eq) 1M hydrazine in THF, |
| | | | absolute EtOH [2.7*10⁻⁴ M], room temperature for 1.5 |
| | | | hours - aldehyde was slowly added to hydrazine |
| | | | Work up: Extraction DCM/H₂O |
| | | | Purification: Recrystallization with MTBE/Hexane |
| 4 | 1b | 1830-003B¹ | 0.2 g SM, 5.0 mL (3.0 eq) 1M hydrazine in THF, |
| | | | absolute EtOH [2.7*10⁻⁴ M], room temperature for 1.5 |
| | | | hours - aldehyde was slowly added to hydrazine |
| | | | Work up: Extraction DCM/H₂O |
| | | | Purification: Extra heating with hydrazine monohydrate |
| 5 | 1b | 1830-004 | 0.2 g SM, 5.0 mL (3.0 eq) 1M hydrazine in THF, THF |
| | | | anhydrous [2.0*10⁻⁴ M], room temperature for 1.5 hours - |
| | | | aldehyde was added to hydrazine with syringe pump |
| | | | Work up: extraction DCM/H₂O |
| 6 | 1b | 1830-006 | 0.2 g SM, 5.0 mL (3.0 eq) 1M hydrazine in THF, THF |
| | | | anhydrous [1.6*10⁻⁴ M], 0° C. for 45 minutes - aldehyde |
| | | | was added to hydrazine with syringe pump |
| | | | Work up: extraction DCM/H₂O |
| 7 | 1b | 1830-007 | 0.864 g SM, 4.7 mL 50-60% hydrazine monohydrate |
| | | | [0.28M], ethanol/H₂O, 100° C. - aldehyde was mixed |
| | | | with hydrazine in flow reactor [flow: 2 mL/min] |
| | | | Work up: extraction DCM/H₂O |
| | | | Purification: Distillation |
| 8 | 1b | 1830-011 | Phenylacetaldehyde (1.5 g, 1.45 mL, 12.5 mmol), |
| | | | hydrazine monohydrate (3.13 g, 3.0 mL, 62.4 mmol), |
| | | | 100° C., 1 h |
| | | | Work up: extraction chloroform/H₂O |
| 9 | 2 | 1830-016 | Azine (Batch 1830-014, 0.49 g, 2.1 mmol) hydrazine |
| | | | monohydrate (1.0 g, 1.0 mL, 20.7 mmol), ethanol (1.5 |
| | | | mL), 87° C., 1 h |
| | | | Work up: extraction chloroform/H₂O |
| 10 | 2 | 1830-018 | Azine (Batch 1830-013, 0.3 g, 1.2 mmol), hydrazine |
| | | | monohydrate (3.05 g, 3.0 mL, 60.9 mmol), ethanol (1.5 |
| | | | mL), 85° C., 1 h |
| | | | Work up: extraction chloroform/H₂O |

¹Crude material was split and used for method A or B purification method.

TABLE 6B

| No. | Amount | Purity¹ | Yield² | Comments |
|---|---|---|---|---|
| 1 | Not | — | — | Similar to UPLC profiles |
| | isolated | | | for reactions carried out in |
| 2 | Not | — | — | the reflux and the room |
| | isolated | | | temperature - the azine is |
| | | | | a main product |
| 3 | F* = 155 mg | UPLC (reaction | F* = 33% | F* = filtrate |
| | P** = 6 mg | mixture): 90% PEH | P** = 1.5% | P** = precipitate |
| | | NMR (after | | |
| | | purification): | | |
| | | F* = 48% | | |
| | | P** = 57% | | |
| 4 | 0.110 g | UPLC (reaction | 40% | — |
| | | mixture): 100% | | |
| | | PEH | | |
| | | NMR (after | | |
| | | purification): 82% | | |
| 5 | 0.278 g | UPLC (reaction | 67% | — |
| | | mixture): 85% PEH | | |
| | | NMR (after | | |
| | | purification): | | |
| | | PEH = 54% | | |
| | | Azine: 44% | | |
| 6 | 0.291 g | UPLC (reaction | 93% | — |
| | | mixture): 100% | | |
| | | PEH | | |
| | | NMR (after | | |
| | | purification): | | |
| | | PEH = 72% | | |
| | | Azine = 25% | | |
| 7 | 0.850 g | UPLC (reaction | 73% | — |
| | | mixture): 100% | | |
| | | PEH | | |
| | | NMR (after | | |
| | | purification): | | |
| | | PEH = 74% | | |
| | | Azine = 26% | | |
| 8 | 1.210 g | UPLC (crude): 25% | | NMR performed in |
| | | contamination with | | deuterated CAN, py, CDCl₃, |
| | | azine | | MeOD, C₆D₆, THF-d₈ |
| | | NMR: depends on | | |
| | | deuterated solvent | | |
| | | used (90-97%) | | |
| 9 | 0.450 g | UPLC (crude): | 63% | — |
| | | 100% PEH | | |
| | | NMR: | | |
| | | PEH = 77% | | |
| | | Azine = 23% | | |
| 10 | 0.313 g | UPLC: 100% of | 80% | — |
| | | PEG (last analysis) | | |
| | | NMR: | | |
| | | PEH = 80% | | |
| | | Azine = 20% | | |

¹UPLC was performed in basic conditions (acidic - decomposition of PEH)
²Yield was estimated acc. to ¹H NMR spectrum.

### (i) Synthesis of 1830-006 (Step 1B)

To the solution of hydrazine (1M in THF, 5.0 mL, 0.005 mol) in the anhydrous THF (4.0 mL) a solution of phenylacetaldehyde (0.2 g, 0.2 mL, 0.0017 mol) in anhydrous THF (1.0 mL) was added with syringe pump (0.1 mL/1 min) at 0° C. to the reaction water (15 mL) was added and the product was extracted with DCM (3×10 mL). The organic layer was washed with water (10 mL) and dried (anhydrous K₂CO₃) and the solvent was removed in vacuo to afford 1830-006.

### (ii) Synthesis of 1830-018 (Step 2)

To the azine (1830-013-p, 0.3 g, 1.2 mmol) solution in ethanol (1.5 mL) hydrazine monohydrate (3.05 g, 3.0 mL, 60.9 mmol) was added. The mixture was then refluxed for 1 h. Water was added to the cooled mixture and the product was extracted with chloroform (3×10 mL). The chloroform layer was washed with water (15 mL), dried over anhydrous potassium carbonate and filtered. The filtrate was evaporated (15 min, 35° C.) and the obtained yellowish oil was dried under vacuum (15 min).

### (iii) Analysis of Reaction Conditions

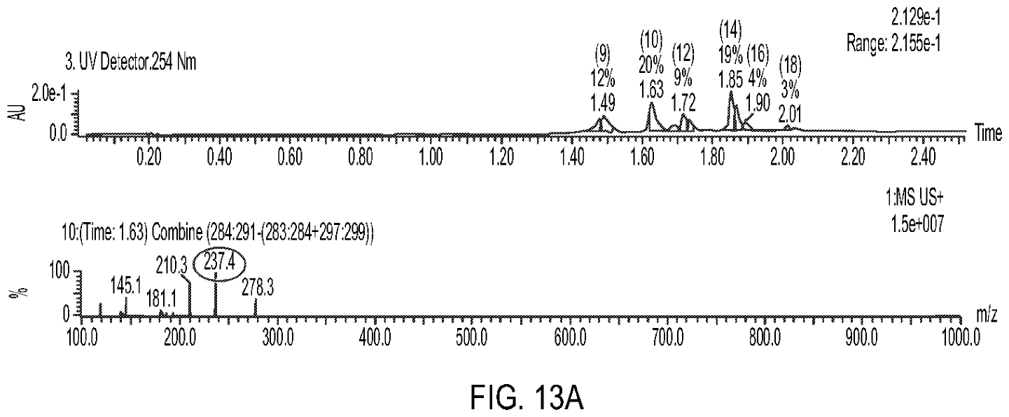

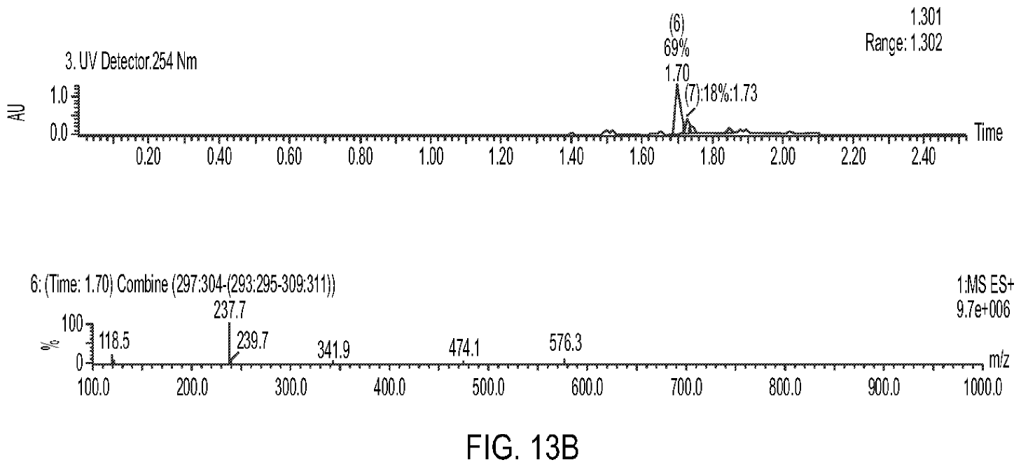

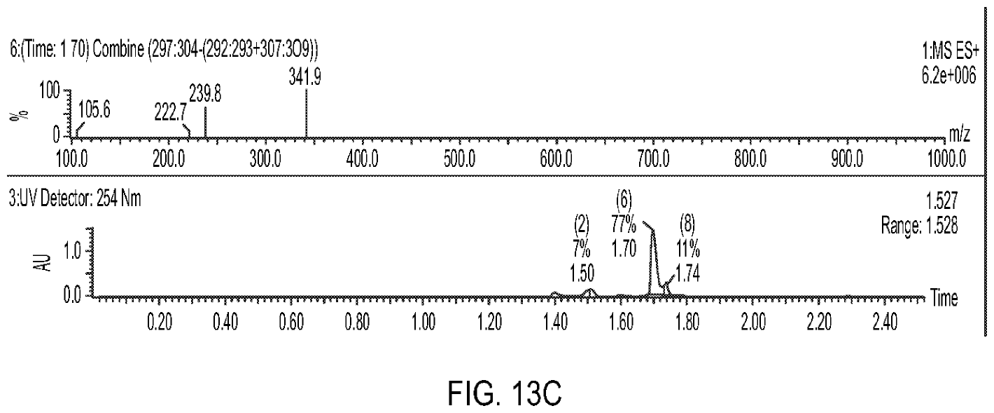

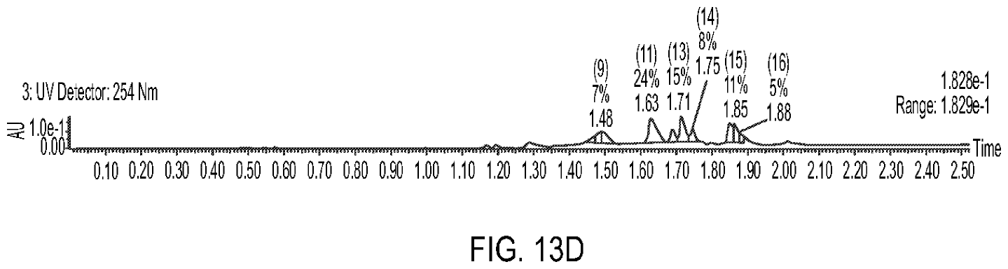

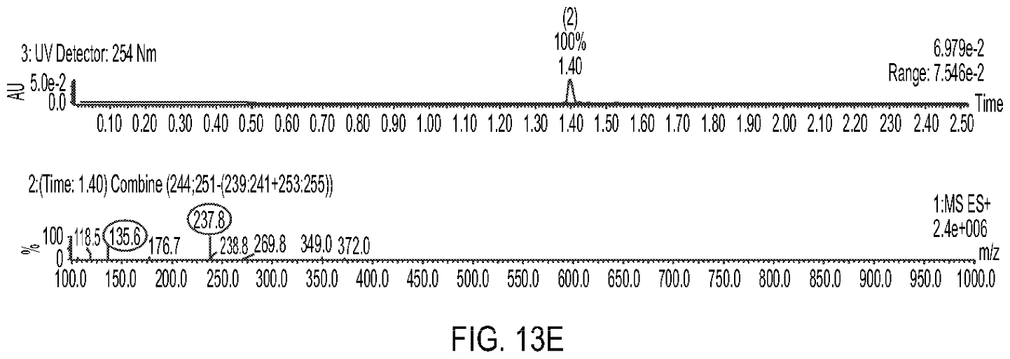

Batch 1830-001. The chromatogram in acidic conditions for the product obtained in 1830-001 is shown in FIG. 13A, top panel. Although the peak at retention time of 1.63 min was confirmed to correspond to the azine via the mass spectrum (FIG. 13A, bottom panel, ESI⁺[M+H]⁺ 237.4), without wishing to be bound by theory, the presence of impurities suggests that the product is not stable in acidic conditions. The purity of the sample was 20%.

The chromatogram in basic conditions for sample 1830-001 is shown in FIG. 13B, top panel. The peak at R.T.=1.70 min corresponds to the azine according to mass spectrometry (FIG. 13B, bottom panel), and the azine was present at 69% purity. Without wishing to be bound by theory, this suggests that the product is stable in basic conditions.

Batch 1830-002. The chromatogram obtained for sample 1830-002 and the corresponding mass spectrum are shown in FIG. 13C, top and bottom panels, respectively. The peak at R.T.=1.70 min corresponds to the azine (77% purity).

Batch 1830-003A and 1830-003B. FIG. 13D is the chromatogram obtained for sample 1830-003 in acidic conditions. No peaks were observed for PEH or the azine. FIG. 13E (top panel) is the chromatogram of reaction 1830-003 in basic conditions. A single peak of 100% purity corresponding to PEH (FIG. 13E, bottom panel, [M+H]⁺ 135.6) was observed at R.T. 1.40 min. The mass spectrum (FIG. 13E, bottom panel) showed molecular ion peaks corresponding to both PEH and the azine, [M+H]⁺ 135.6 and 237.8, respectively, indicating that the azine was likely formed during the electrospray ionization process.

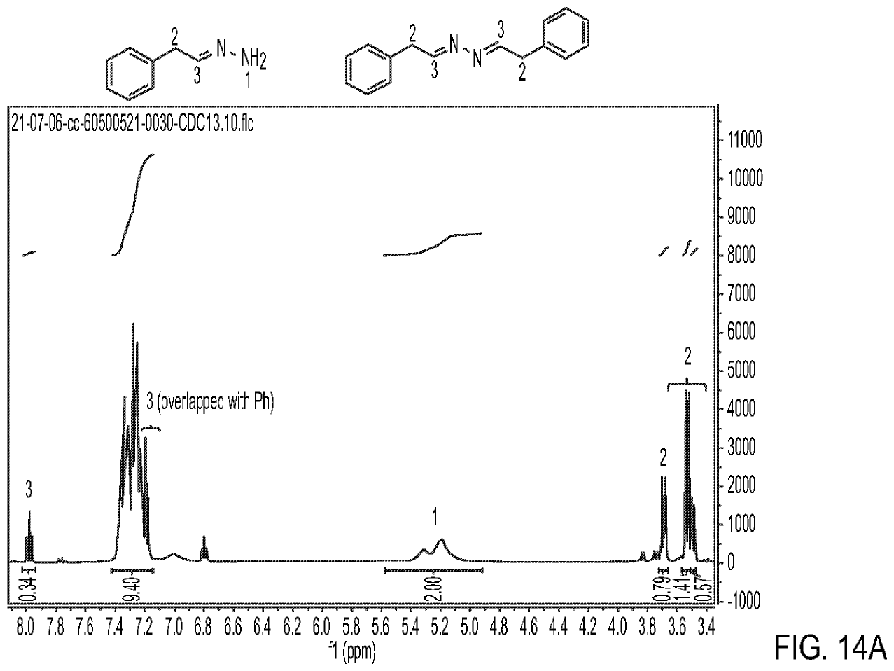

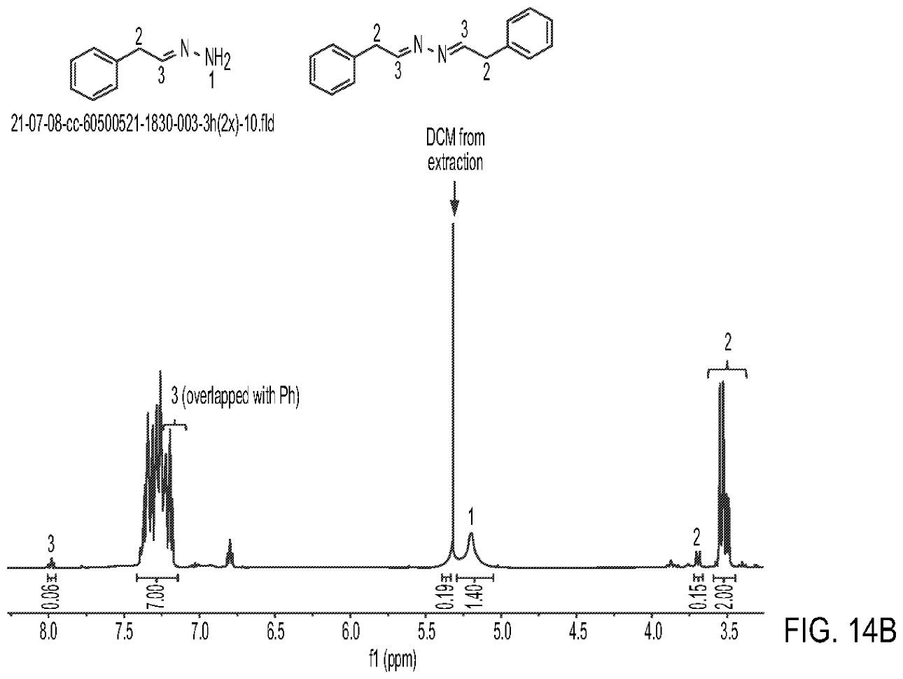

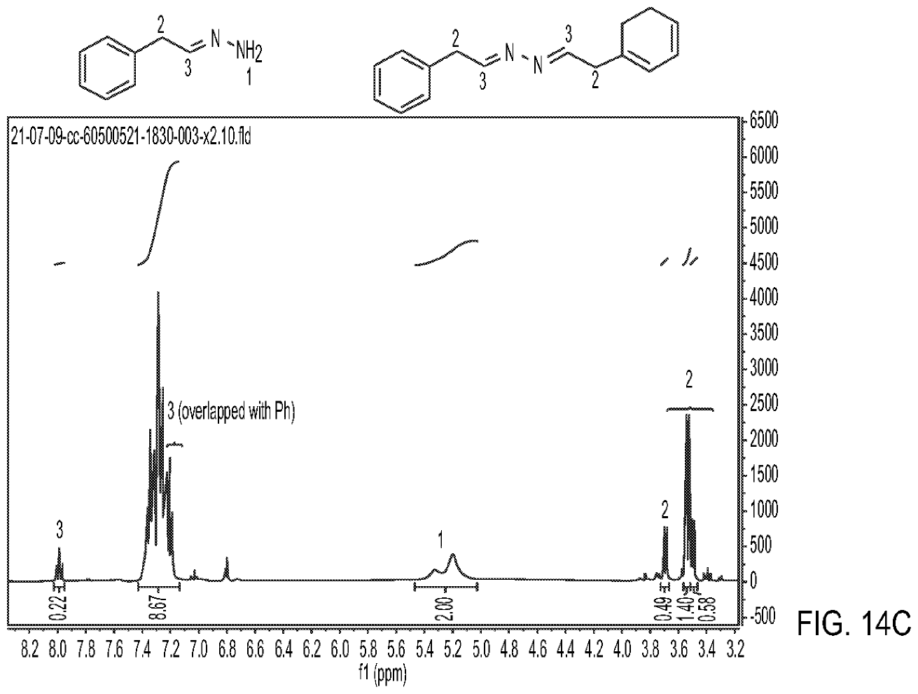

As noted above, reaction 1830-003 was worked up using two different methods: (1) extraction with dichloromethane/H₂O and recrystallization in methyl tert-butyl ether (A) (1830-003A); and (2) heating at 100° C. with NH₂NH₂·H₂O (1830-003B). The ¹H NMR spectrum obtained after method A is shown in FIG. 14A. Signals corresponding to both PEH and the azine were observed, and the azine comprised 26% of the mixture according to the NMR integration analysis. FIG. 14B and FIG. 14C show the NMR spectra after method B, where FIG. 14B is after drying for 15 min and FIG. 14C is after drying for 3 hours. During the drying process, the level of azine impurity increased from 7% to 22% for 15 min and 3 hours, respectively. Without wishing to be bound by theory, this suggests that PEH can react with a second PEH molecule to form the azine, and that PEH may be volatile.

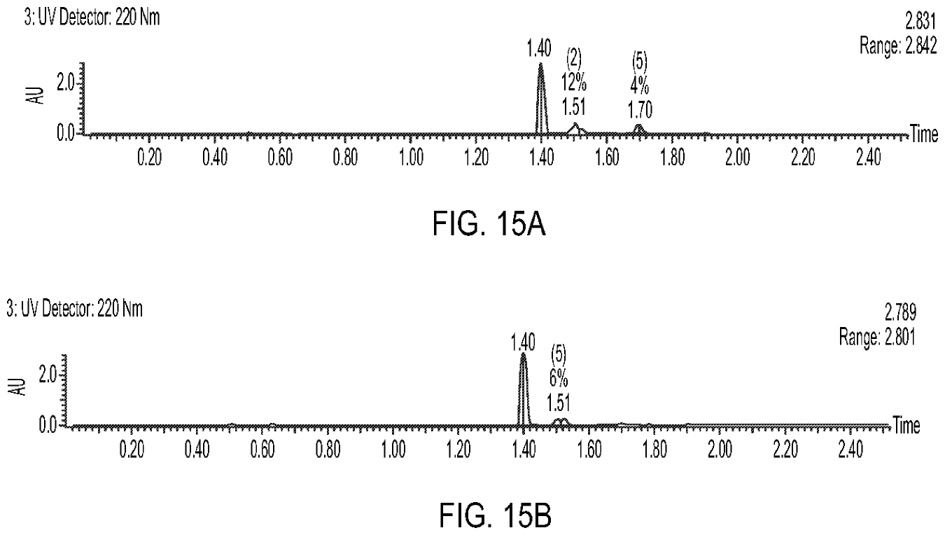

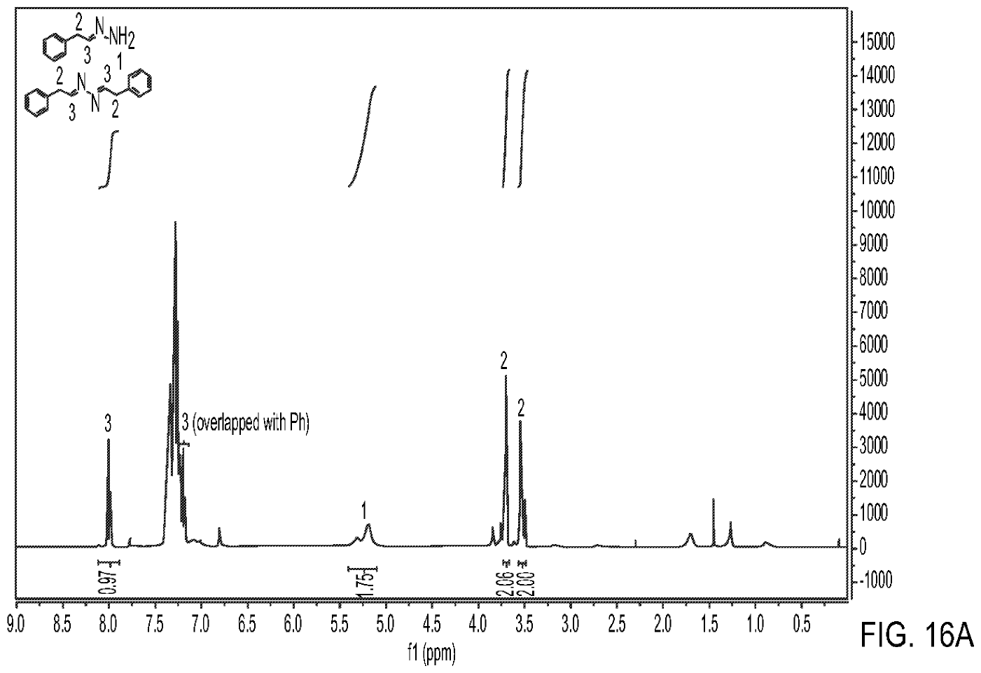

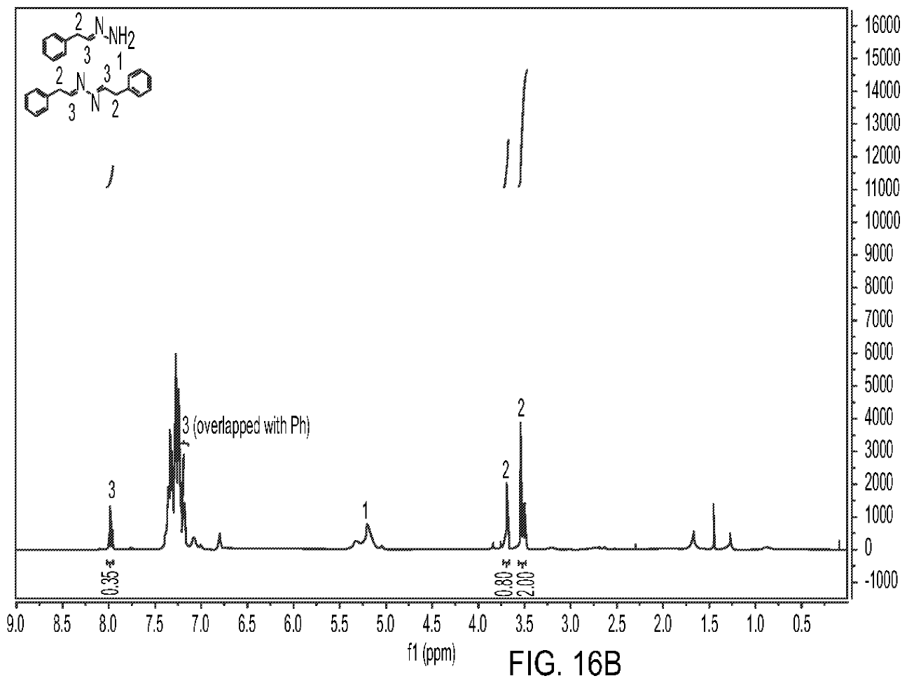

Batch 1830-004 and 1830-006. FIG. 15A and FIG. 15B show the UPLC chromatograms obtained for samples 1830-004 and 1830-006, respectively. The corresponding ¹H NMR spectrums are shown in FIG. 16A (1830-004) and FIG. 16B (1830-004).

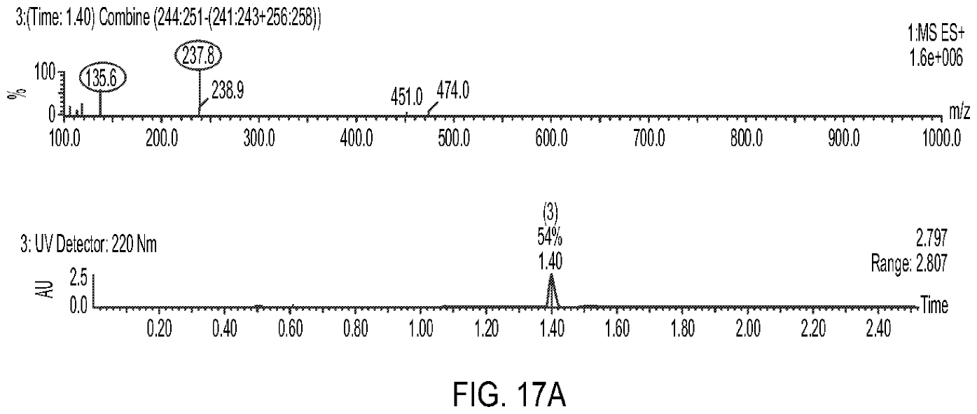

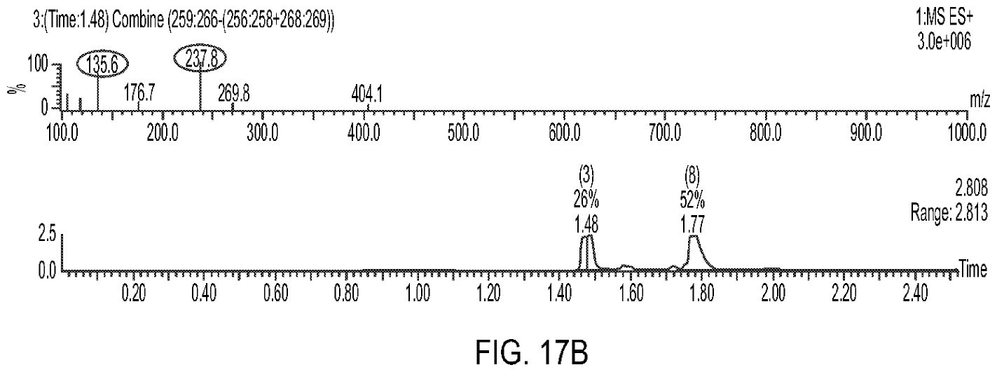

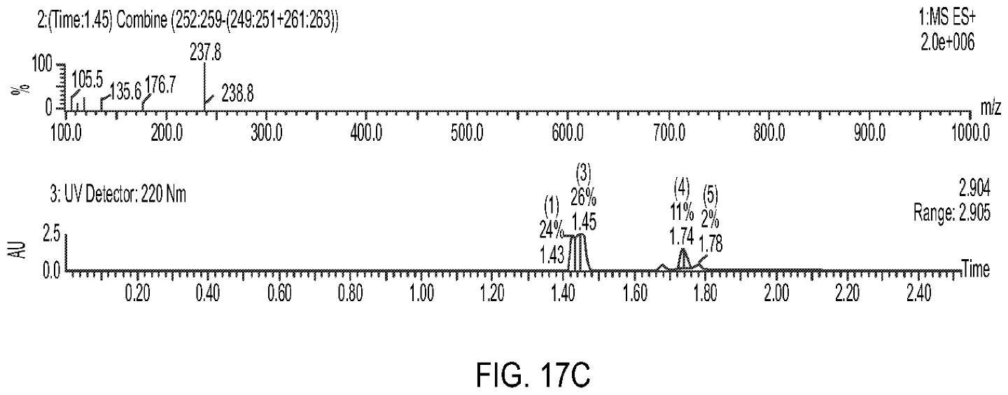

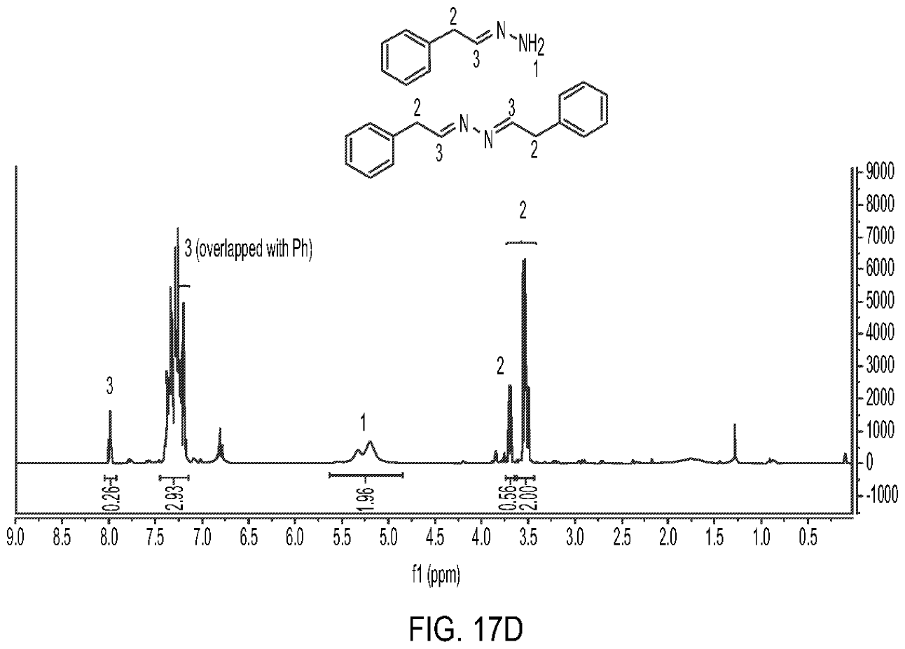

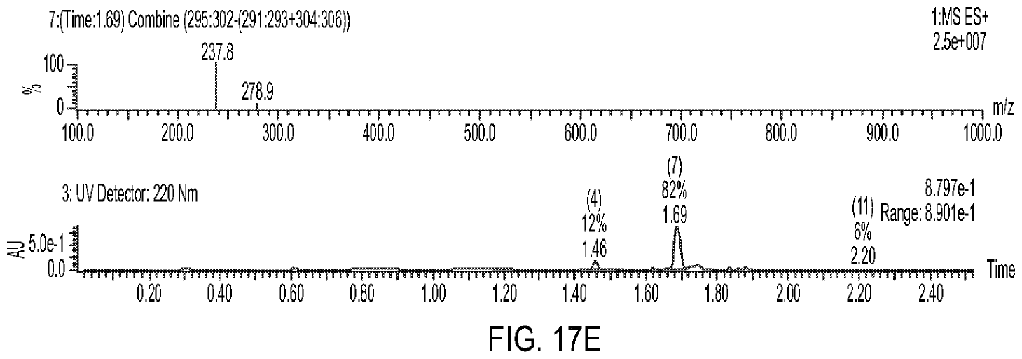

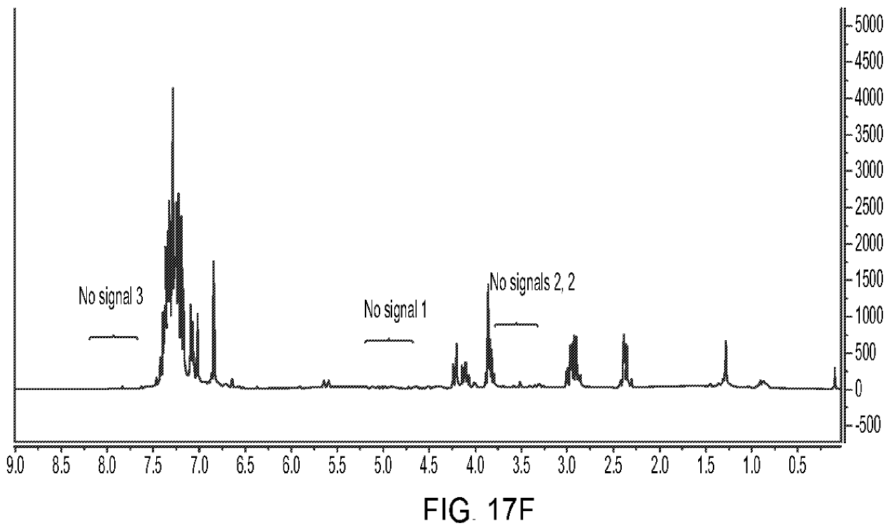

Batch 1830-007. Sample 1830-007 was subjected to purification by distillation. The reaction was worked up by evaporating volatile EtOH and diluting the crude product with water. The product was extracted with DCM (3×50 mL). The combined organic layers were washed with water 3× (100 mL) and brine, and the product was dried over sodium sulphate to give 850 mg of product as yellow oil. This mixture of azine and PEH was distilled under reduced pressure. Two fractions were collected: Fraction 1 (Fr1, 157 mg mass, T=110-120° C., Pressure=0.12 bar) and Fraction 2 (Fr2, 318 mg mass, T=160-170° C., Pressure=0.12 bar). The mass spectrum (top panel) and UPLC chromatogram (bottom panel) of the resultant material is shown in FIG. 17A. A single peak on the chromatogram was observed at R.T. 1.40 min, and mass spectrometry revealed peaks for the masses of both PEH and the azine. Without wishing to be bound by theory, the azine was likely formed during the ionization process. FIG. 17B shows the mass spectrum (top panel) and chromatogram (bottom panel) before distillation and after storage at −20° C. under Ar for 3 days. Two peaks are observed in the chromatogram, and the peak at R.T. 1.77 min contained unidentified masses at 239.7 and 341.9 m/z. Without wishing to be bound by theory, this suggests that PEH decomposed during storage at −20° C. The results for Fr1 after distillation are shown in FIG. 17C and FIG. 17D, with the NMR results being measured at a maximum of 40 min after distillation. PEH was detected at 26% purity, and the azine was detected at 11% purity. The results for Fr2 after distillation are shown in FIG. 17E and FIG. 17F, with the NMR results being measured at a maximum of 40 min after distillation. Although the azine was detected via mass spectrometry (FIG. 17E, top panel), no signals corresponding to PEH or the azine were observed in the NMR (FIG. 17F). Without wishing to be bound by theory, the lack of signals corresponding to PEH and the azine in Fr2 suggests that azine and PEH underwent decomposition at 160-170° C.

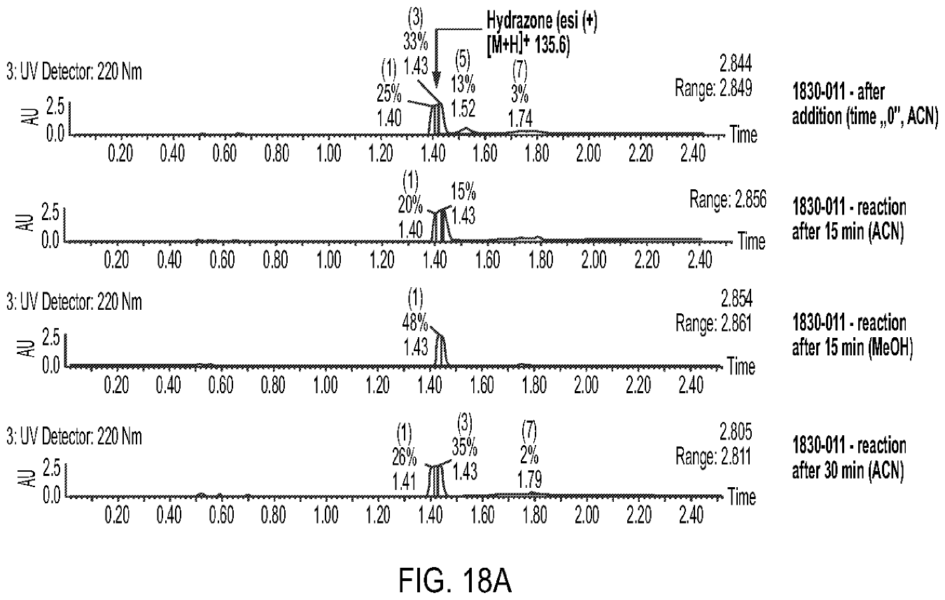

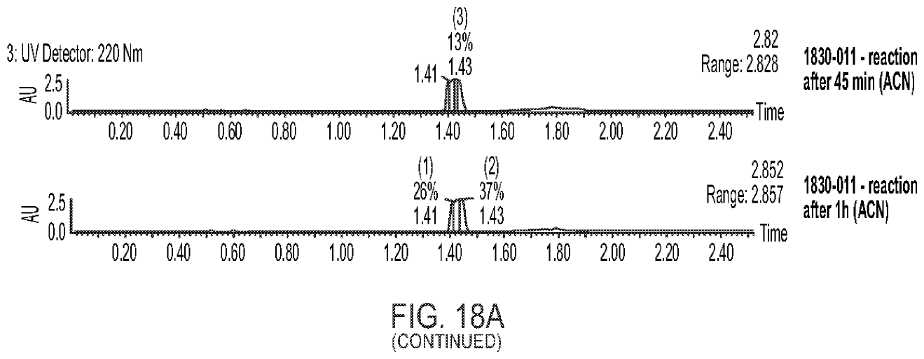

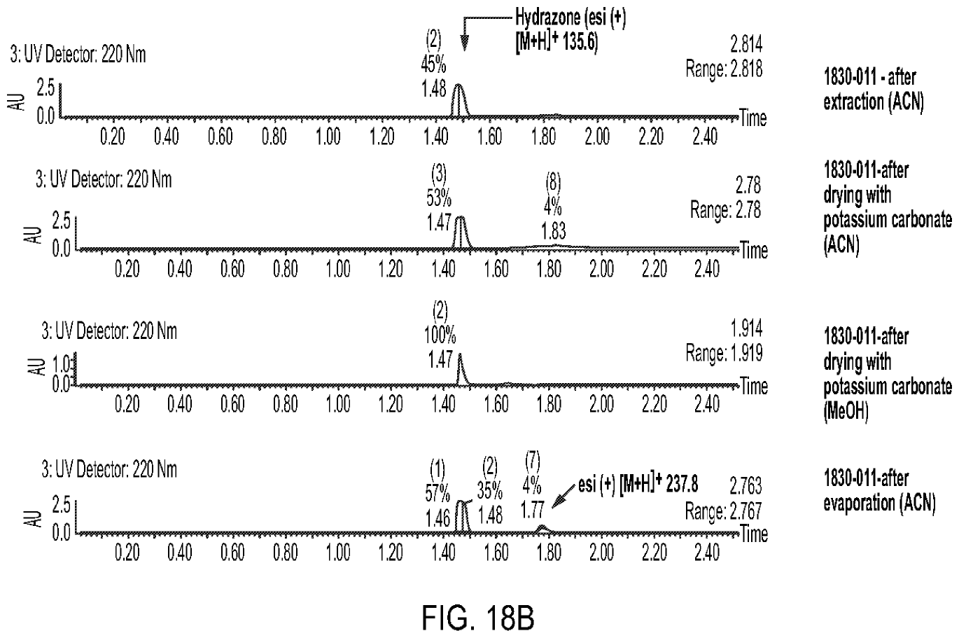

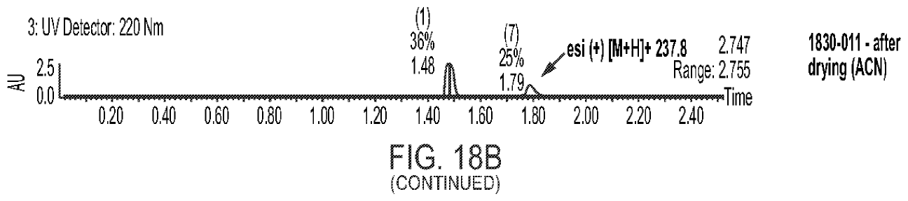

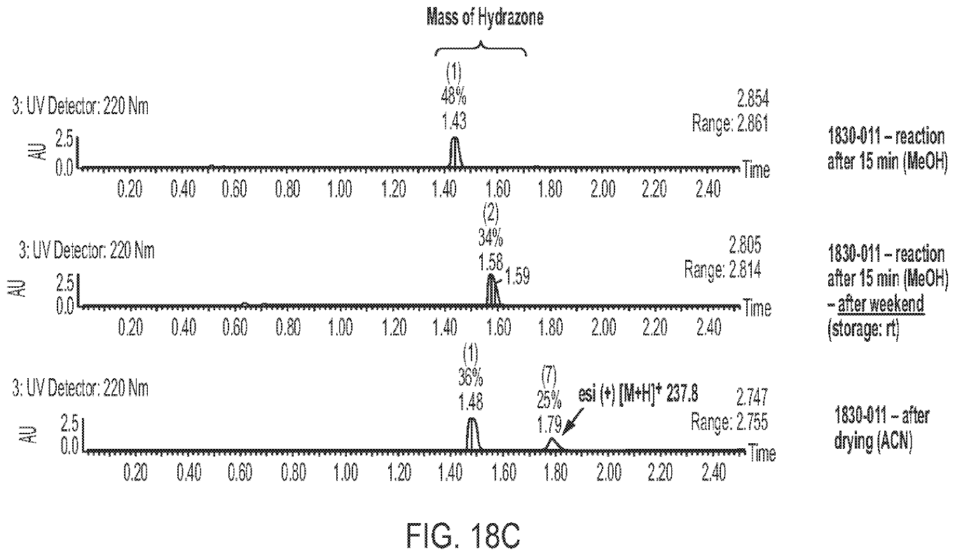

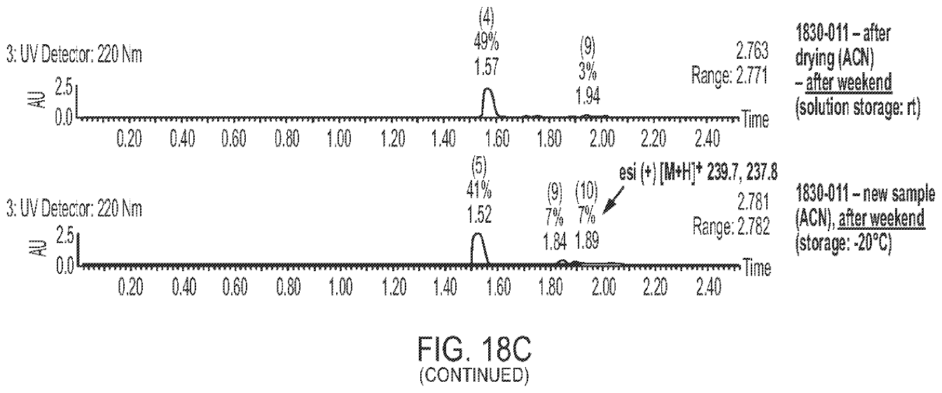

Batch 1830-011. UPLC chromatograms for PEH sample 1830-011 were measured in ACN or MeOH directly after preparation. FIG. 18A-C are chromatograms of sample 1830-011 obtained at different points during the reaction. As shown in FIG. 18A, PEH was detected at every time point. After evaporation and drying (FIG. 18B, bottom two panels), azine was detected ([M+H]⁺=237.8). The chromatograms in FIG. 18C of this species were obtained after 3 days storage at room temperature and −20° C.

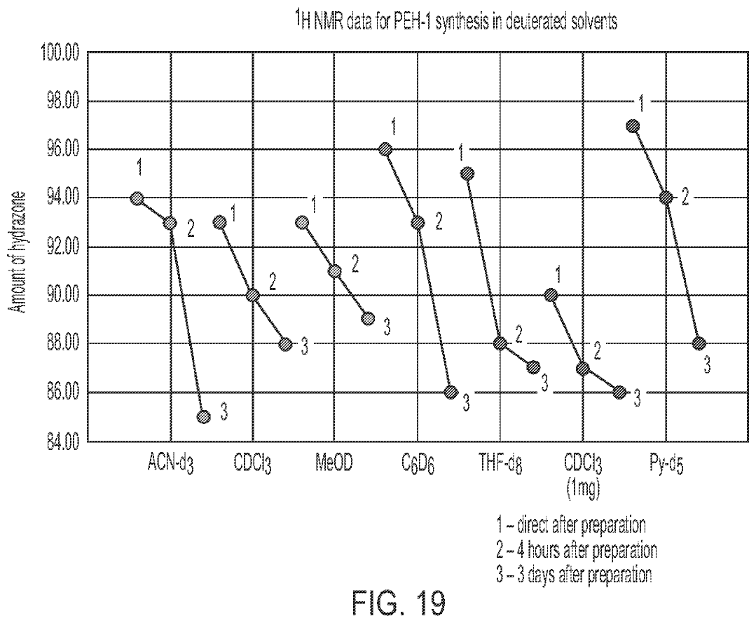

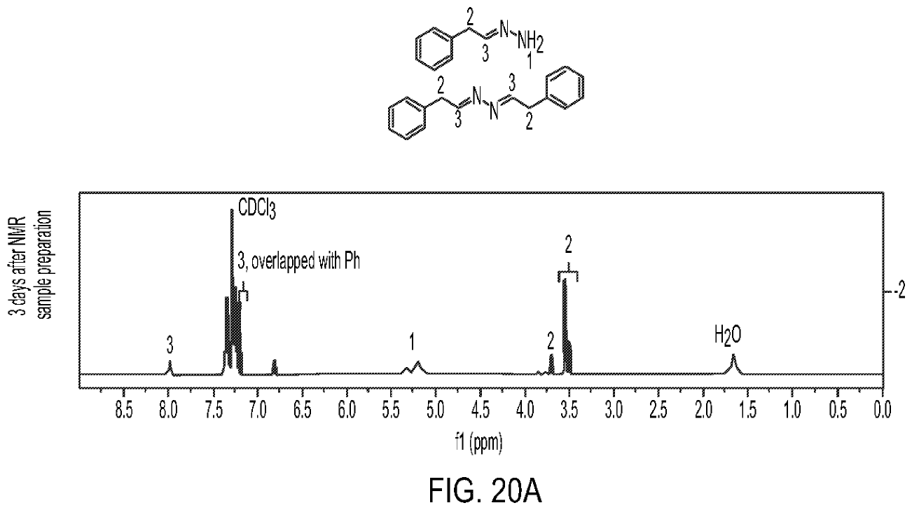

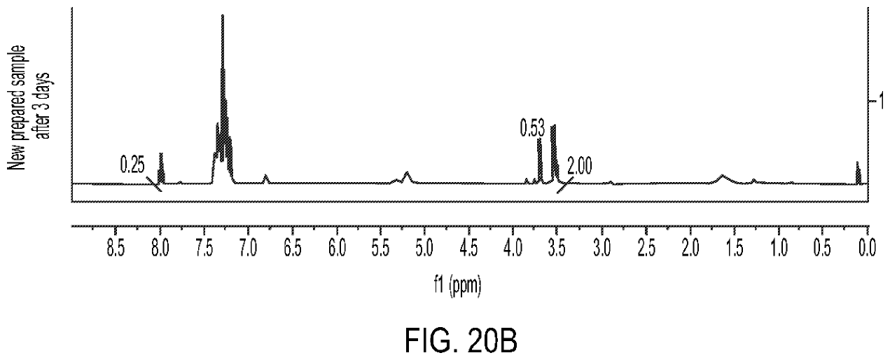

¹H NMR was employed to probe the stability of PEH sample 1830-011 both in terms of time after preparation and in different deuterated solvents. The results are summarized in Table 7 below and FIG. 19. The highest change of PEH:Azine ratio was observed for C₆D₆, ACN-d₃, THF-d₈, and Py-d₅. In Py-d₆, about 10% of PEH was transformed to the azine. Additionally, a new sample from the obtained oil in sample 1830-011 in which the oil was prepared after 3 days storage in −20° C. under argon was prepared and measured directly in CDCl₃. These spectra are shown in FIG. 20A and FIG. 20B. After 3 days, the contamination of PEH with azine was equal to 18% (the ratio of PEH:Azine was 82:18).

TABLE 7

| | | PEH:Azine | | |
|---|---|---|---|---|
| Deuterated | Mass of | Direct after | 4 h after | 3 days after |
| solvent | Sample (mg) | preparation | preparation | preparation |
| ACN-d₃ | 3.2 | 96:4 | 93:7 | 85:15 |
| CDCl₃ | 3.1 | 93:7 | 90:10 | 88:12 |
| MeOD | 3.1 | 93:7 | 91:9 | 89:11 |
| C₆D₆ | 3.1 | 96:4 | 93:7 | 86:14 |
| THF-d₈ | 3.0 | 95:5 | 88:12 | 87:13 |
| CDCl₃ | 1.0 | 90:10 | 87:13 | 86:14 |
| Py-d₅ | 3.0 | 97:3 | 94:6 | 88:12 |

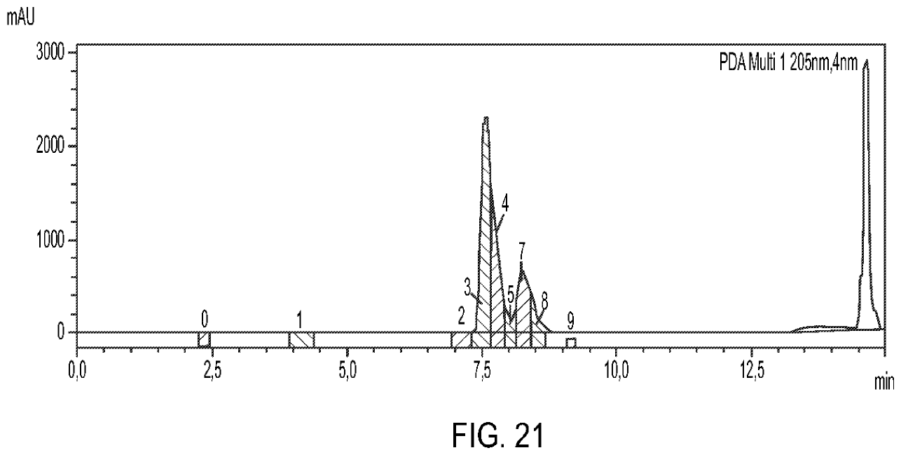

Preparative HPLC was performed on 20.5 mg of solid sample in 0.4 mL acetonitrile on a reverse phase C18 column with a mobile phase of H₂O+0.05% NH₃ and acetonitrile. The method duration was 15 minutes, and the purification duration was 1 hour. The chromatogram obtained from the preparative HPLC purification is shown in FIG. 21, and shows the two desired peaks at 7.6 min (first fraction) and 8.3 min (second fraction). The pump parameters were as described in Table 8 below.

TABLE 8

| | |
|---|---|
| Mode | Binary gradient |
| Pump A | LC-20 AP |
| Pump B | LC-20 AP |
| Total Flow | 30.00 mL/min |
| B. Conc. | 20.0% |
| B Curve | 0 GPa |
| Maximum Pressure | 42.0 MPa |
| Minimum Pressure | 0 MPa |
| Solenoid Valve B Name | FCV-200AL |
| Solenoid Valve B | A |
| Compressibility Setting | On |
| Pump A Compressibility | 0.45/GPa |
| Pump B Compressibility | 0.45/GPa |

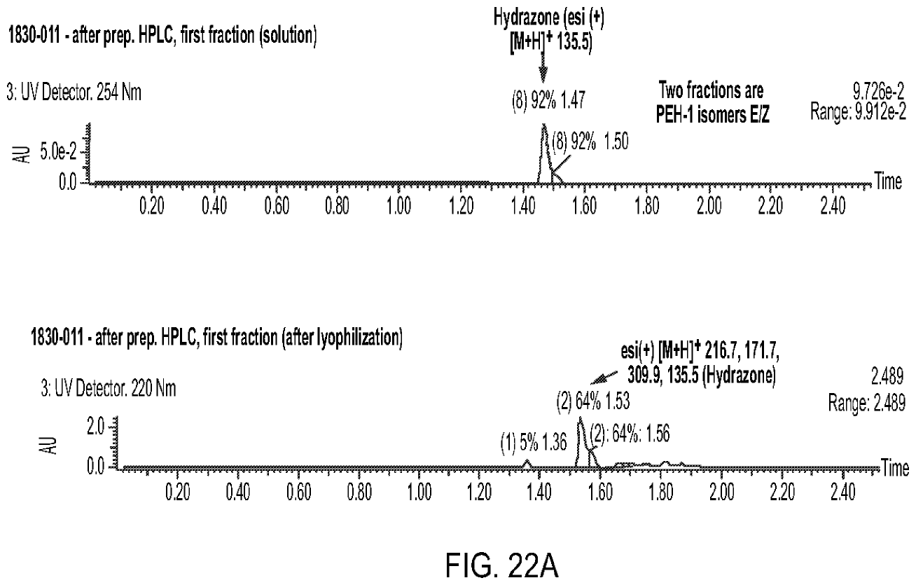

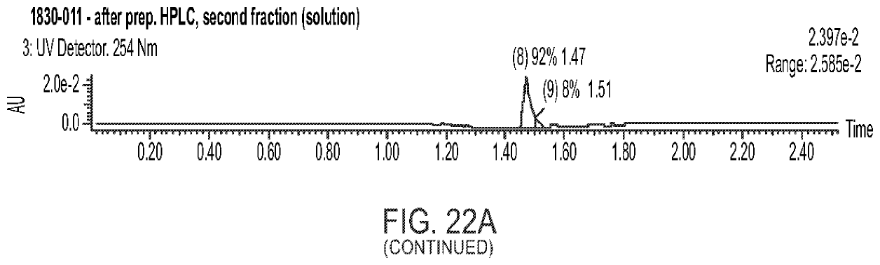

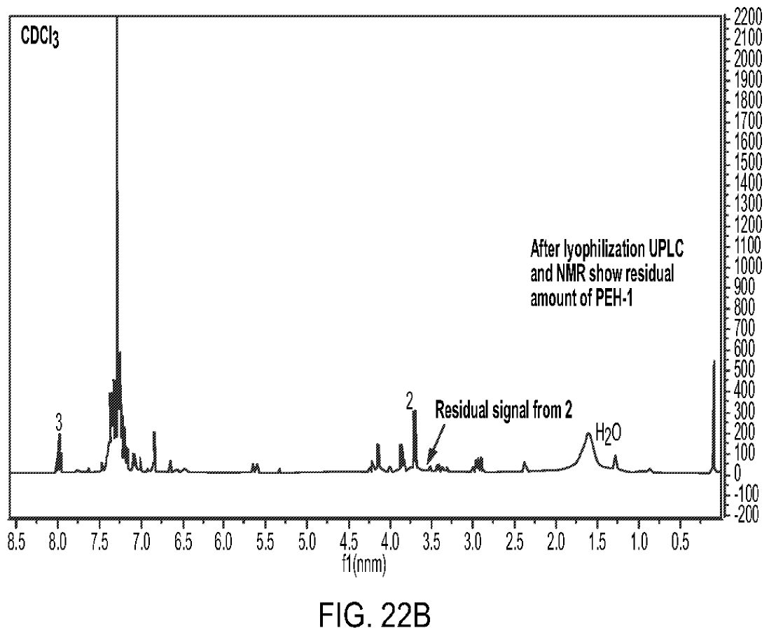

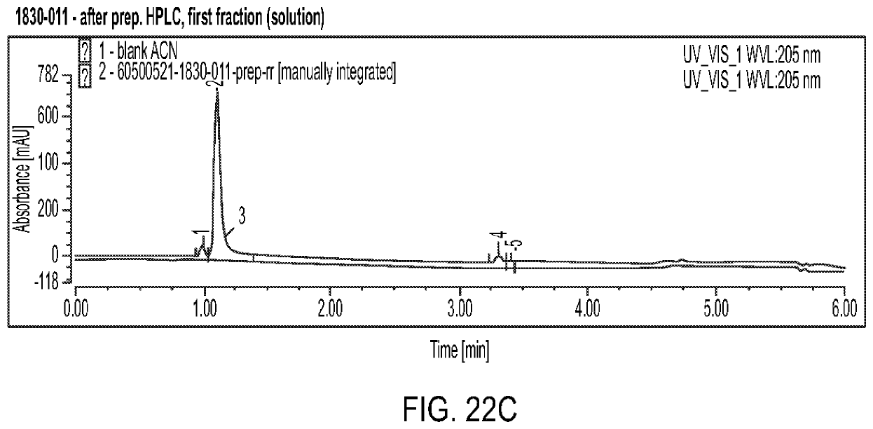

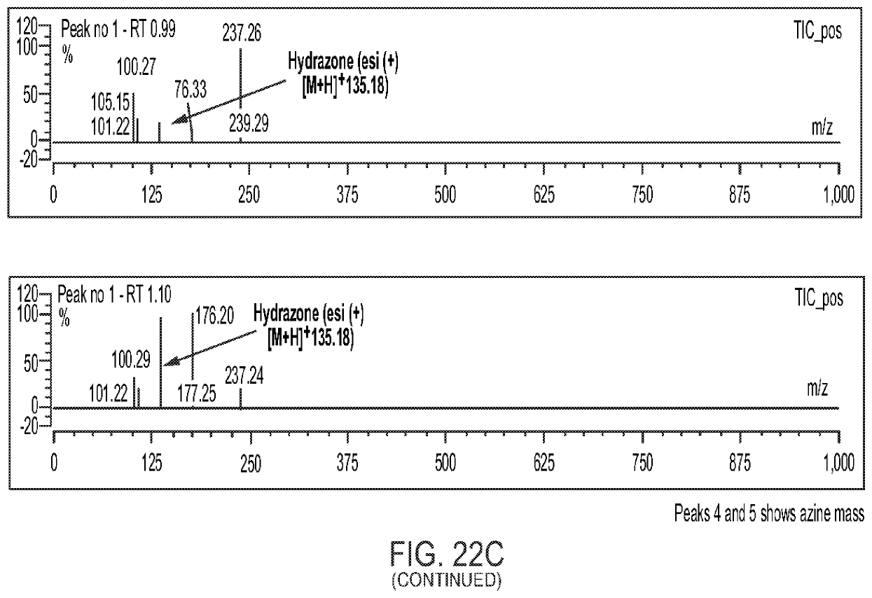

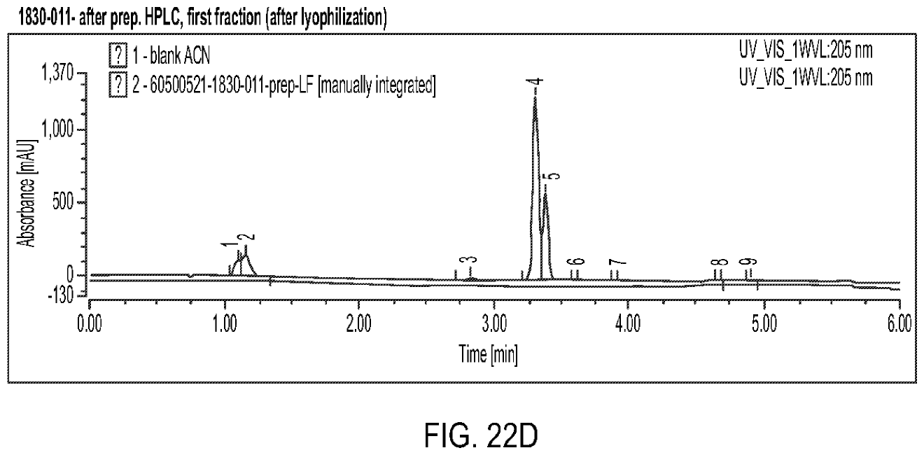

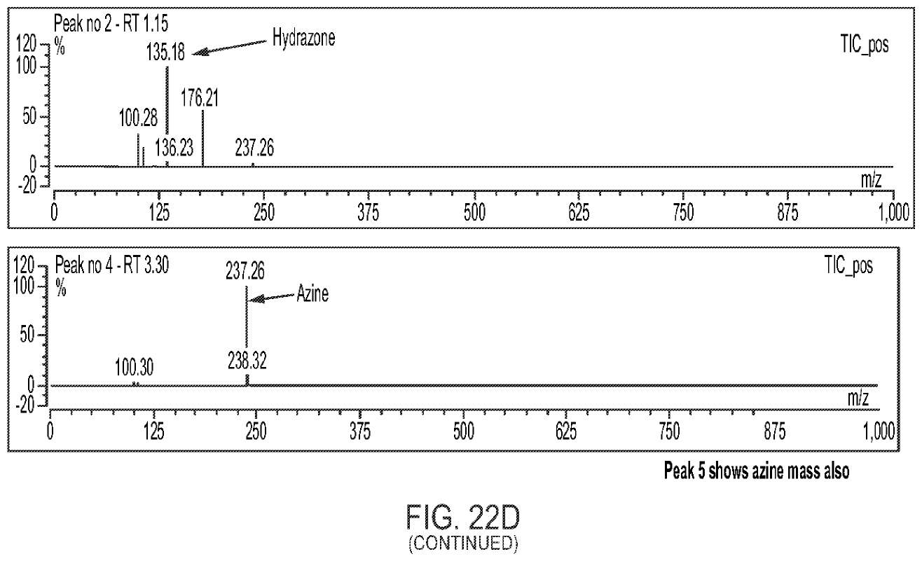

UPLC analysis of 1830-011 showed 100% of PEH after prep-HPLC (solution, LCMS: 92.66% PEH, before lyophilization) (FIG. 22A, top panel). After lyophilization the UPLC profile changed—no mass of the azine was observed (LCMS: 12.70% PEH, after lyophilization) (FIG. 22A, middle panel). NMR analysis (after lyophilization) revealed no signals from PEH (FIG. 22B). Without wishing to be bound by theory, it is believed that PEH undergoes decomposition during lyophilization and the azine is formed. See also FIG. 22A-C. Table 9a corresponds to FIG. 22B, top panel, and shows the analysis of the first fraction in solution after preparative HPLC. Peak nos. 1 and 2 correspond to PEH (92.66%) and peak nos. 4 and 5 correspond to azine (4.14%). Table 9b corresponds to FIG. 22C, top panel, and shows the analysis of the first fraction after preparative HPLC and lyophilization. Peak nos. 1 and 2 correspond to PEH (12.70%) and peak nos. 4 and 5 correspond to azine (85.89%).

TABLE 9A

| | Time | Area | Height | Relative |
|---|---|---|---|---|
| No. | (min.) | (mAU*min) | (mAU) | Area (%) |
| 1 | 0.990 | 1.928 | 44.577 | 4.53 |
| 2 | 1.103 | 37.491 | 706.661 | 88.13 |
| 3 | 1.167 | 1.360 | 23.158 | 3.20 |
| 4 | 3.307 | 1.515 | 30.952 | 3.56 |
| 5 | 3.383 | 0.246 | 5.212 | 0.58 |

| | |
|---|---|
| Total | 100.00 |

TABLE 9B

| | Time | Area | Height | Relative |
|---|---|---|---|---|
| No. | (min.) | (mAU*min) | (mAU) | Area (%) |
| 1 | 1.097 | 4.932 | 108.047 | 4.60 |
| 2 | 1.150 | 8.686 | 146.499 | 8.10 |
| 3 | 2.820 | 1.182 | 16.783 | 1.10 |
| 4 | 3.303 | 60.651 | 1250.869 | 56.53 |
| 5 | 3.380 | 31.501 | 595.878 | 29.36 |
| 6 | 3.613 | 0.081 | 1.554 | 0.08 |
| 7 | 3.913 | 0.040 | 1.036 | 0.04 |
| 8 | 4.677 | 0.043 | 1.328 | 0.04 |
| 9 | 4.903 | 0.171 | 4.841 | 0.16 |

| | |
|---|---|
| Total | 100.00 |

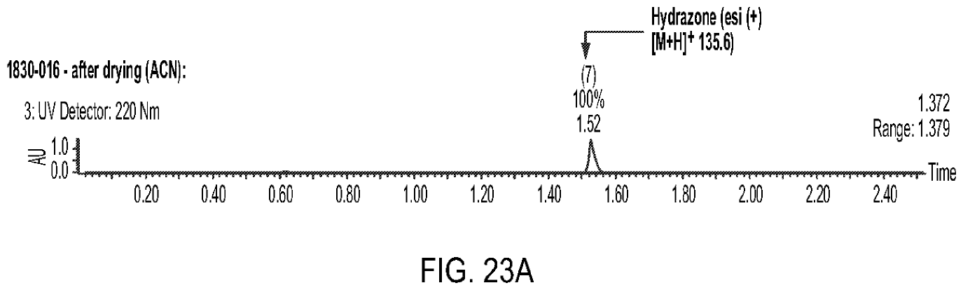

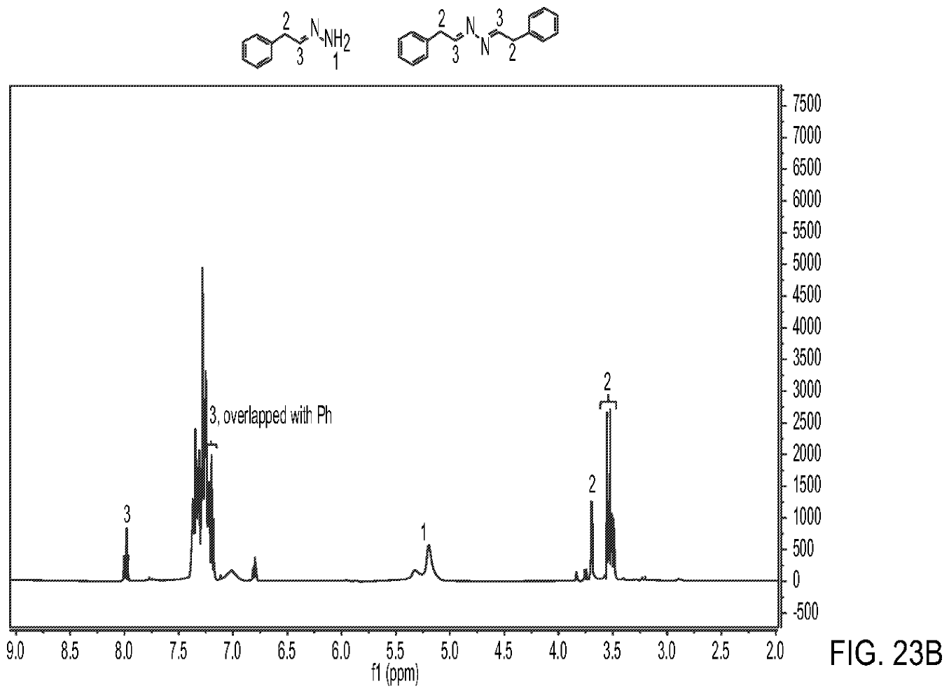

Batch 1830-016 The chromatogram obtained for the PEH product formed in 1830-016 in ACN after drying is shown in FIG. 23A. Pure PEH was detected ([M+H]⁺=135.6). The corresponding ¹H NMR spectrum is shown in FIG. 23B.

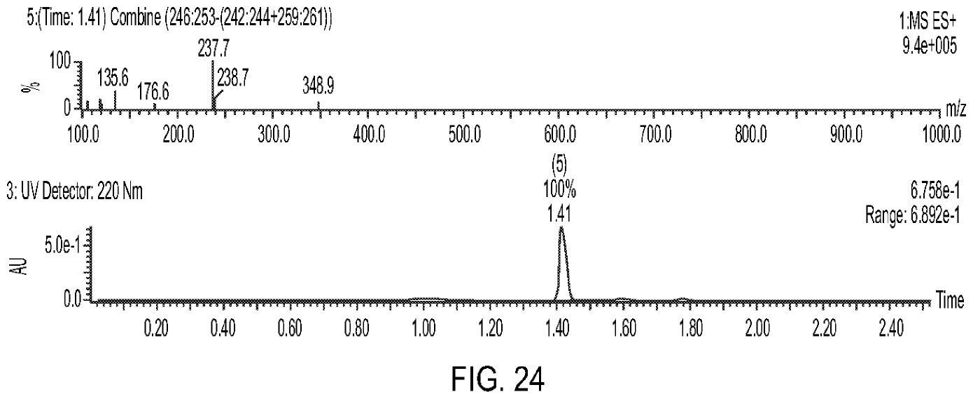

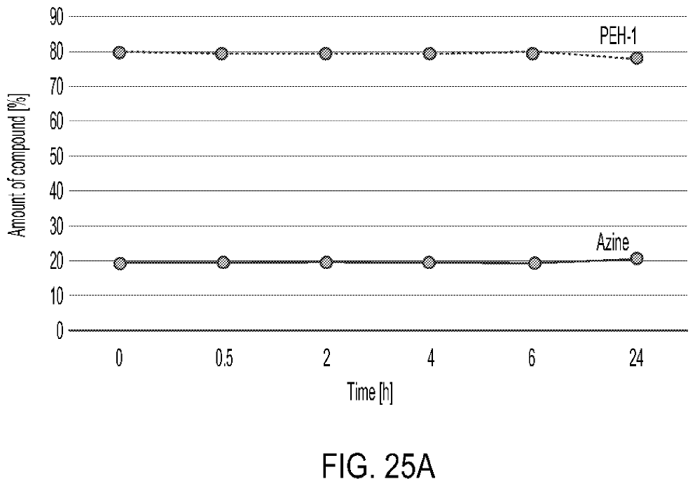

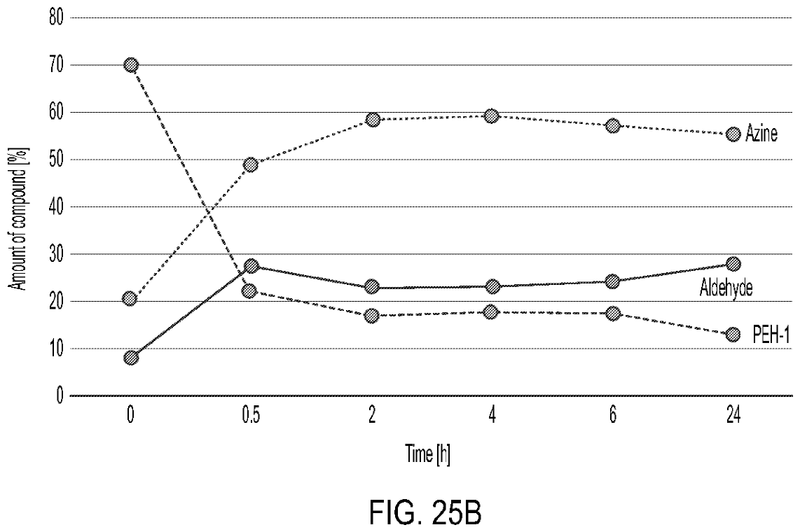

Batch 1830-018. The mass spectrum analysis and chromatogram (after extraction, before evaporation) obtained for the PEH product formed in 1830-018 is shown in FIG. 24 (top and bottom panels, respectively). pH stability studies demonstrated that PEH is stable at pH=9.2. See FIG. 25A, demonstrating the amount of PEH and azine at pH 9.2 after dissolution of PEH. No aldehyde was detected, indicating that hydrolysis does not occur at the higher pH. At pH=7.4, the azine and the aldehyde are formed. See FIG. 25A, which shows the distribution of PEH, azine, and aldehyde at pH 7.4 over time.

c. Analytics

Unless stated otherwise, all exemplary data pertaining to UPLC and LCMS chromatography reported in Method 1 above were obtained using the methods detailed below.

UPLC Method. The UPLC analysis was performed on a Waters ACQUITY UPLC I-Class PLUS System with a Waters SQ Detector 2 in the wavelength range of 200-400 nm. Compounds were separated on an Acquity UPLC BEH C18 1.7 μm/130 Å (2.1×100 mm) column using gradient elution with a column temperature of 40° C. at a flow of 0.5 mL/min. The elution scheme shown in Table 11 was used for chromatographic separation. Mobile phase A was 0.05% ammonium hydroxide in water (28.0-30.0% NH₃ basis) and Mobile phase B was acetonitrile. The syringe washing solution was 50% ACN, 50% water. The MS conditions were a mass range of 100 and scan time of 0.15 s.

TABLE 11

| | Mobile | Mobile | |
|---|---|---|---|
| Time | phase A | phase B | Flow |
| (min) | (%) | (%) | (mL/min) |
| 0.00 | 80 | 20 | 0.5 |
| 0.10 | 80 | 20 | 0.5 |
| 1.10 | 0 | 100 | 0.5 |
| 2.00 | 0 | 100 | 0.5 |
| 2.50 | 80 | 20 | 0.5 |
| 3.00 | 80 | 20 | 0.5 |

LCMS Method. The LCMS analysis was performed on a Dionex UHPLC Ultimate 3000 with DAD detector/Thermo Scientific ISQ EC-Mass Spectrometer with a wavelength range of 190-350 nm±4 nm. Compounds were separated on a Kinetix® 2.6 μm XB—C18 (4.6×50 mm), 110 A, column no. 00B-4496-EO column using gradient elution with a column temperature of 25° C. at a flow of 1.0 mL/min. The elution scheme shown in Table 12 was used for chromatographic separation. Mobile phase C was 0.05% ammonium hydroxide in water (28.0-30.0% NH₃ basis) and Mobile phase D was acetonitrile. The syringe washing solution was 20% MeOH. The MS conditions were a mass range of 100-1000 m/z and scan speed of 12 000 amu/sec.

TABLE 12

| | Mobile | Mobile | |
|---|---|---|---|
| Time | phase C | phase D | Flow |
| (min) | (%) | (%) | (mL/min) |
| 0.00 | 70 | 30 | 1.0 |
| 3.35 | 20 | 80 | 1.0 |
| 3.75 | 20 | 80 | 1.0 |
| 3.90 | 5 | 95 | 1.0 |
| 4.75 | 5 | 95 | 1.0 |
| 5.00 | 70 | 30 | 1.0 |
| 6.00 | 70 | 30 | 1.0 |

### 2. Synthesis of PEH and Azine (Method 2)

A synthetic scheme for the azine and PEH is shown below.

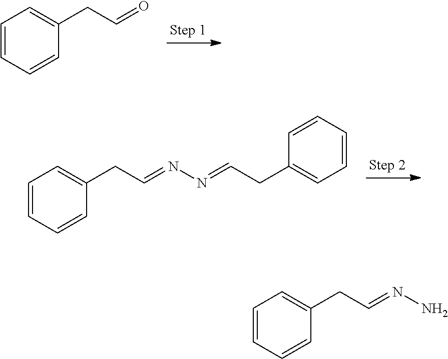 [SMILES: O=CCc1ccccc1 ; C(/Cc1ccccc1)=N\N=C\Cc1ccccc1 ; N/N=C/Cc1ccccc1]

a. Step 1: Azine Formation

Reaction conditions for Batches 018-022 are shown in Table 13a and the results shown in Table 13b below.

TABLE 13A

| No. | Batch | Conditions |
|---|---|---|
| 1 | 1830- | Phenylacetaldehyde (20.0 g, 18.54 mL, 16.65 mmol), |
| | 018 | ethanol (80.0 mL), hydrazine monohydrate (4.17 g, 4.17 |
| | | mL, 8.32 mmol), reflux (89° C.), 1 h. |
| | | Work up: Addition of water, extraction with chloroform |
| | | Purification: Trituration 19 g of crude with cold ethanol |
| | | at −5° C. -> 480 mg of yellow product, which was |
| | | crystallized with EtOH |
| 2 | 1830- | Phenylacetaldehyde (20.0 g, 18.54 mL, 16.65 mmol), |
| | 019 | ethanol (80.0 mL), hydrazine monohydrate (4.17 g, 4.17 |
| | | mL, 8.32 mmol), reflux, 1 h. |
| | | Work up: Addition of water, extraction with chloroform |
| | | Purification: 1 g of crude was dissolved at ~80° C. then |
| | | kept at −~10° C.; 1 g of crude was dissolved at RT then |
| | | kept at −~10° C.; Crystallization from 16 g of crude |
| | | combined with 1830-020 |
| 3 | 1830- | Phenylacetaldehyde (9.71 g, 9.0 mL, 8.08 mmol), |
| | 020 | ethanol (40.0 mL), hydrazine monohydrate (2.02 g, 2.02 |
| | | mL, 4.04 mmol), reflux, 2 h |
| | | Work up: Addition of water, extraction with chloroform |
| | | Purification: Crystallization from 8 g |
| | | combined with batch 1830-019 |
| 4 | 1830- | Phenylacetaldehyde fresh* (60.0 g, 55.61 mL, 49.94 |
| | 021 | mmol), ethanol (240.0 mL), hydrazine monohydrate |
| | | (12.50 g, 12.50 mL, 24.97 mmol), reflux, 1 h |
| | | Work up: Addition of water, extraction with chloroform |
| | | Purification: After combining with 1830-022 |
| | | crystallization from 400 mL of ethanol. Starting from |
| | | RT and keeping in freezer ON in aprox. −25° C. |
| 10 | 1830- | Phenylacetaldehyde fresh* (60.0 g, 55.61 mL, 49.94 |
| | 022 | mmol), ethanol (240.0 mL), hydrazine monohydrate |
| | | (12.50 g, 12.50 mL, 24.97 mmol), reflux, 1 h |
| | | Work up: Addition of water, extraction with chloroform |
| | | Purification: After combining with 1830-021 |
| | | crystallization from 400 mL of ethanol. Starting from |
| | | RT and keeping in freezer ON in aprox. −25° C. |

*320 g of phenylphenylacetaldehyde was distilled under vacuum 4.5*10−2 bar, 36-37° C., 290 g after distillation - ¹H NMR confirmed, synthesis of azine from freshly distilled aldehyde gives product with higher purity

TABLE 13B

| | | Purity/ | | |
|---|---|---|---|---|
| No. | Amount | LCMS | Yield | Comments |
| 1 | 0.380 g | 99.48% | 54% before | Purity comes from |
| | | | purification | summarized purities of |
| | | | | isomers at 205 nm. |
| | | | | Product was used as SM in |
| | | | | 1830-023. |
| | | | | ~18 g of filtrate material |
| | | | | left. |
| 2 | 11.88 g | 87.33% | — | 620 mg after crystallization |
| | | | | (94% purity by UPLC). |
| | | | | 853 mg after crystallization |
| | | | | at RT then (94% purity by |
| | | | | UPLC). |
| 3 | | | | Reaction was set up only to |
| | | | | check if longer time of |
| | | | | reaction will have an |
| | | | | impact on the yield. It did |
| | | | | not. |
| 4 | 90 g | 98.84% | 38% | Purity comes from |
| 5 | | | | summarized purities of |
| | | | | isomers at 205 nm. |

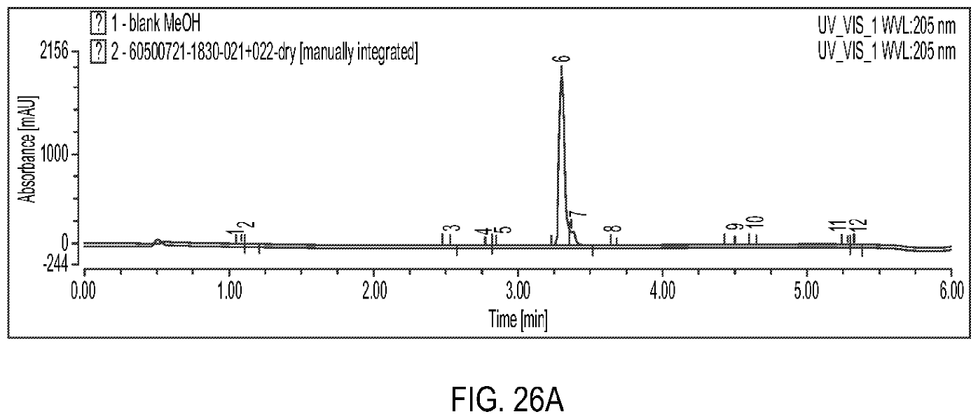

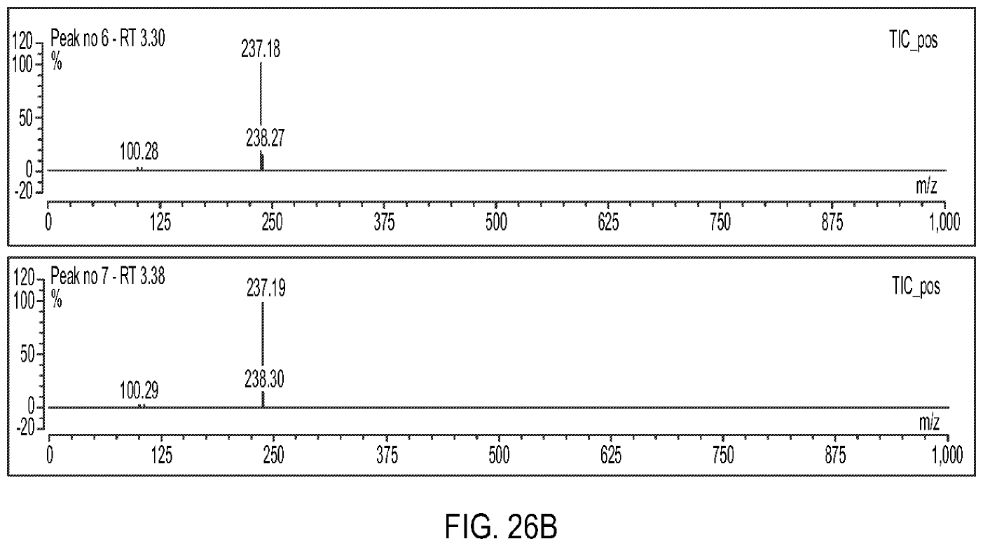

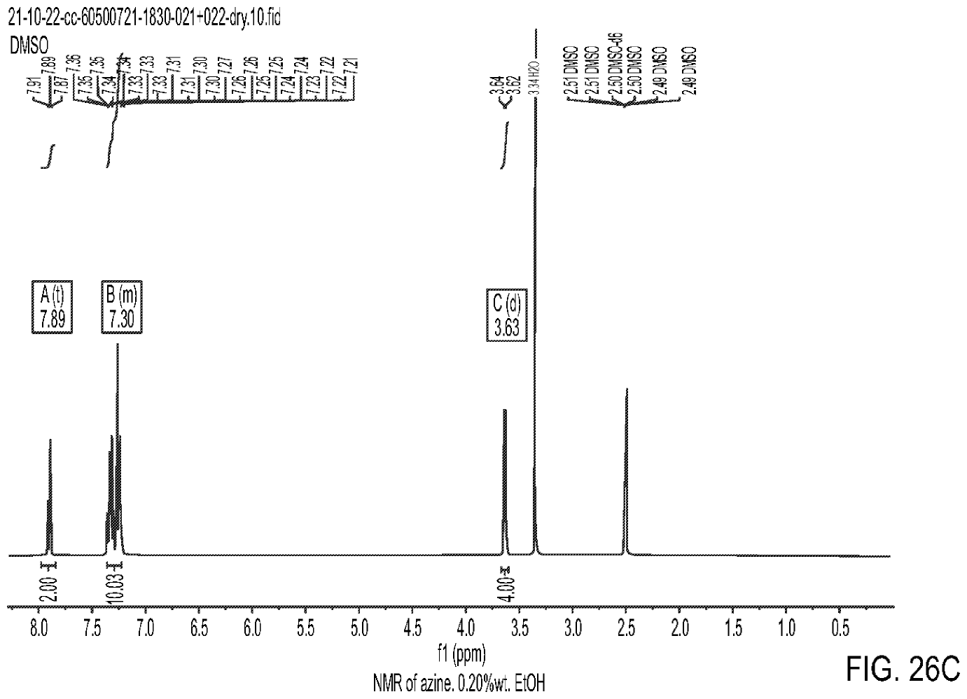

1830-021 and 1830-022. LCMS analysis of the azine product found 89.56% purity for the first isomer (r.t.=3.30 min, 205 nm, ESI (+) [M+H]⁺=237.18) and 9.32% purity for the second isomer (r.t.=3.38 min, 205 nm, ESI (+) [M+H]⁺=237.19). See also FIG. 26A and FIG. 26B. A ¹H NMR spectrum in DMSO-d₆ of the azine in 0.20 wt % EtOH is shown in FIG. 26C (¹H NMR (300 MHz, DMSO-d₆) δ 7.89 (t, J=5.8 Hz, 2H), 7.36-7.21 (m, 10H), 3.63 (d, J=5.8 Hz, 4H)).

b. Step 2: PEH Formation

Reaction conditions for Batch 1830-023 are shown in Table 14a and the results shown in Table 14b and Table 14c below. See also FIG. 27, which illustrates the results from the stability tests of PEH.

TABLE 14A

| No. | Batch | Conditions |
|---|---|---|
| 1 | 1830- | Azine batch 1830-018 (0.380 g, 1.61 mmol), ethanol |
| | 023 | (1.90 mL), hydrazine monohydrate (4.07 g, 4.07 mL, |
| | | 81.32 mmol), reflux (89° C.), 1 h |
| | | Work up: Addition of water, extraction with chloroform |
| | | Half of material was concentrated to give 108 mg of |
| | | colourless oil and half of material was not concentrated |
| | | and was left in 30 mL of chloroform. Samples were |
| | | stored at −80° C. |

TABLE 14B

| No. | Amount | Purity | Yield | Comments |
|---|---|---|---|---|
| 1 | Quant. | Stability tests were | Quant. | Purities below comes |
| | | run, purities shown | | from the summarized |
| | | below in Table 14c. | | isomers. |

TABLE 14C

| | After | After 1 | After 2 | After 3 | After 4 | After 7 |
|---|---|---|---|---|---|---|
| State | reaction | day | days | days | days | days |
| Extract | 98.49 | 97.36 | 97.86 | 97.82 | 97.65 | 96.14 |
| (liq) | | | | | | |
| solid | 81.17 | 68.46 | 69.82 | 64.26 | 66.89 | 67.38 |

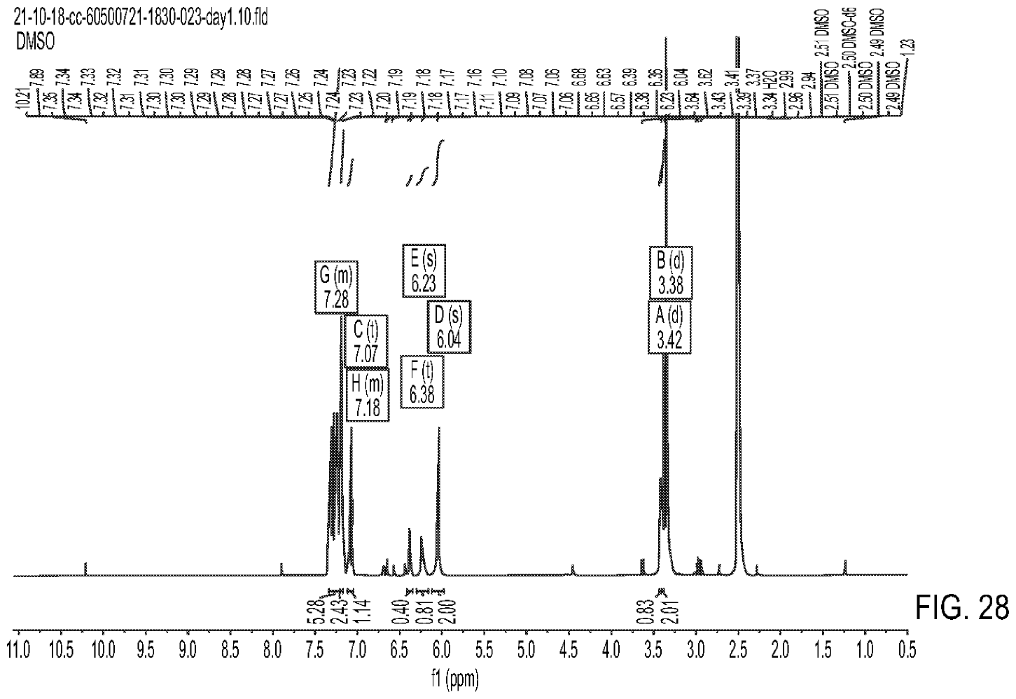

A ¹H NMR spectra of the product PEH in DMSO-d₆ is shown in FIG. 28. ¹H NMR (300 MHz, DMSO-d₆) δ 7.33-7.22 (m, 5H), 7.20-7.17 (m, 2H), 7.07 (t, J=5.7 Hz, 1H), 6.38 (t, J=5.0 Hz, OH), 6.23 (s, 1H, 6.04 (s, 2H), 3.42 (d, J=5.0 Hz, 1H), 3.38 (d, J=5.7 Hz, 2H).

c. LCMS Method

The LCMS analysis used for the PEH stability tests was performed on a Dionex UHPLC Ultimate 3000 with DAD detector/Thermo Scientific ISQ EC-Mass Spectrometer with a wavelength range of 190-350 nm±4 nm. Compounds were separated on a Kinetix® 2.6 μm XB—C18 (4.6×50 mm), 110A, column no. 00B-4496-EO column using gradient elution with a column temperature of 25° C. at a flow of 1.0 mL/min. The elution scheme shown in Table 15 was used for chromatographic separation. Mobile phase C was 0.05% ammonium hydroxide in water (28.0-30.0% NH₃ basis) and Mobile phase D was acetonitrile. The syringe washing solution was 20% MeOH. The MS conditions were a mass range of 100-1000 m/z and scan speed of 12 000 amu/sec.

TABLE 15

| | Mobile | Mobile | |
|---|---|---|---|
| Time | phase C | phase D | Flow |
| (min) | (%) | (%) | (mL/min) |
| 0.00 | 70 | 30 | 1.0 |
| 3.35 | 20 | 80 | 1.0 |
| 3.75 | 20 | 80 | 1.0 |
| 3.90 | 5 | 95 | 1.0 |
| 4.75 | 5 | 95 | 1.0 |
| 5.00 | 70 | 30 | 1.0 |
| 6.00 | 70 | 30 | 1.0 |

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

1. A pharmaceutical composition comprising a therapeutically effective amount of a compound having a structure represented by a formula: 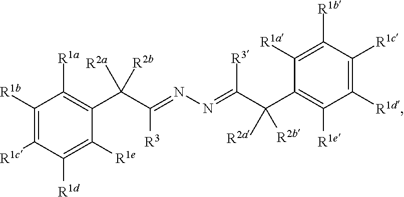
wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein each of R^{2a}, R^{2a′}, R^{2b}, and R^{2b′} is independently selected from hydrogen, halogen, —OH, —NH₂, C1-C4 alkyl, and C2-C4 alkenyl; and
wherein each of R³ and R^{3′} is independently selected from hydrogen and C1-C4 alkyl,
or a pharmaceutically acceptable salt thereof, and a pharmaceutically acceptable carrier.

2. The pharmaceutical composition of claim 1, wherein the compound is laterally symmetric.

3. The pharmaceutical composition of claim 1, wherein each of R^{1a}, R^{1b}, R^{1c}, R^{1d}, R^{1e}, R^{1a′}, R^{1b′}, R^{1e′}, R^{1d′}, and R^{1e′} is hydrogen.

4. (canceled)

5. The pharmaceutical composition of claim 1, wherein each of R^{2a}, R^{2a′}, R^{2b}, and R^{2b′} is hydrogen.

6. The pharmaceutical composition of claim 1, wherein each of R³ and R^{3′} is hydrogen.

7-9. (canceled)

10. The pharmaceutical composition of claim 1, wherein the compound has a structure represented by a formula:

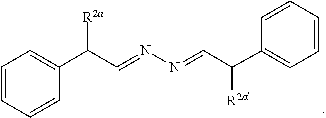

11. The pharmaceutical composition of claim 1, wherein the compound has a structure represented by a formula:

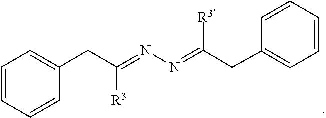

12. The pharmaceutical composition of claim 1, wherein the compound has a structure represented by a formula:

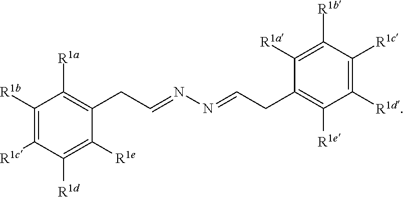

13. The pharmaceutical composition of claim 1, wherein the compound is:

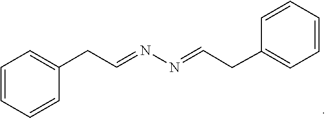 [SMILES: C(/Cc1ccccc1)=N\N=C\Cc1ccccc1]

14. (canceled)

15. The pharmaceutical composition of claim 1, further comprising an effective amount of an antidepressant.

16-22. (canceled)

23. The pharmaceutical composition of claim 1, wherein the composition further comprises a biocompatible polymer.

24. (canceled)

25. The pharmaceutical composition of claim 1, wherein the composition is formulated as an oral dosage form.

26. The pharmaceutical composition of claim 1, wherein the composition is formulated for intranasal administration, transdermal administration, or intradermal administration.

27. (canceled)

28. A pharmaceutical composition comprising a therapeutically effective amount of a compound having a structure represented by a formula: 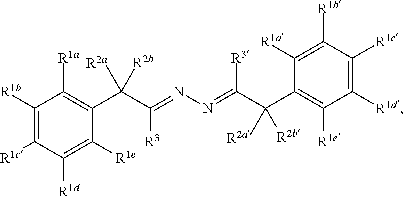
wherein each of Ria R^{1b}, We, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein each of R^{2a}, R^{2a′}, R^{2b}, and R^{2b′} is independently selected from hydrogen, halogen, —OH, —NH₂, C1-C4 alkyl, and C2-C4 alkenyl; and
wherein each of R³ and R^{3′} is independently selected from hydrogen and C1-C4 alkyl,
or a pharmaceutically acceptable salt thereof, and an antidepressant, a biocompatible polymer, and a pharmaceutically acceptable carrier.

29. The pharmaceutical composition of claim 0, wherein the antidepressant is buspirone.

30-35. (canceled)

36. A method for treating a psychological disorder in a subject in need thereof, the method comprising administering to the subject an effective amount of a compound having a structure represented by a formula: 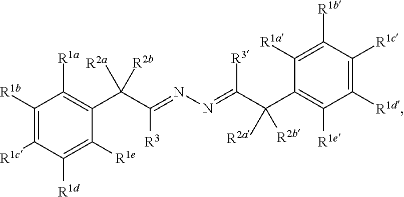
wherein each of R^{1a}, R^{1b}, R^{1c}, R^{1d}, R^{1e}, R^{1a′}, R^{1b′}, R^{1c′}, R^{1d′}, and R^{1e′} is independently selected from hydrogen, halogen, —CN, —NH₂, —OH, —NO₂, C1-C4 alkyl, C2-C4 alkenyl, C1-C4 haloalkyl, C1-C4 cyanoalkyl, C1-C4 hydroxyalkyl, C1-C4 haloalkoxy, C1-C4 alkoxy, C1-C4 alkylamino, (C1-C4)(C1-C4) dialkylamino, and C1-C4 aminoalkyl;
wherein each of R^{2a}, R^{2a′}, R^{2b}, and R^{2b′} is independently selected from hydrogen, halogen, —OH, —NH₂, C1-C4 alkyl, and C2-C4 alkenyl; and
wherein each of R³ and R^{3′} is independently selected from hydrogen and C1-C4 alkyl,
or a pharmaceutically acceptable salt thereof.

37. The method of claim 0, wherein the compound is:

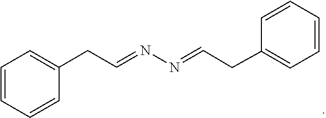 [SMILES: C(/Cc1ccccc1)=N\N=C\Cc1ccccc1]

38. The method of claim 0, wherein the method further comprises administering an antidepressant.

39-49. (canceled)

50. The method of claim 36, wherein the psychological disorder is a severe anxiety disorder, obsessive-compulsive disorder (OCD), or treatment-resistant depression.

51-53. (canceled)

54. The method of claim 36, wherein the subject has not responded to at least one adequate antidepressant treatment prior to the administering step.

55-74. (canceled)
